SEQUENCE LISTING (67 sequences)

SEQ ID NO: 1 - protein, 936 aa, synthetic construct
MSKDGDKMKHSKQEVSEDKGVKKDPSVFRCQNLAILEMREEVADYYADLQADYRHYLPLMILWGQNGFMEPDETGLNFSAVLPSTAKTRCENRYGAQIAADLKLIADDLPSSLYAIFTNDYQPLTGVKKVGDRWKFVTSLNTVSDEEFAKKVKPLLRDYKGVTEEQWRRFREERVAGKTADQVWDDLDSILRNPTLAALKKIHDKHMFRVKRPESKLYCDALAHMLSSSYDSWKKINEARQLELQAMRDEQQGLLANKTTNSLYNLVCEFADELESQNYGLTRRIFFIARKEWEDEKSHYLPVIKLLVTEKYLPLLQADNKTVSPAVKAWDICSTLKQRSPYIRFVPPTKDYPIPFGQTGRGHKFTLKEEAGKVCFLVPGLTKDEMPLSGSHYFSSLRIDKVGTDFKISFRHKTKIRSKKSARVTVKQPRINGTVTEVGILRRNGKFFLRVAYKIAYPQHNIDLKSYFASAAPSTQKLELLPAQMRVAGVDLNISDPIVVSKADIFKGEDGGPLSVLDYGSGKVVGEPVIICKDTSRAGKISSLATKCRTLRDVIRNFRHSLHQGTPLPSKSLEFLQEVPVAQEDLKSPSPRYLIQTWIKYIRTEMKRLHYKVWLDGYCHTSEAMRMLDLVDQVAFLMKSYENIHVKPGHKIPKKTTASLKIRETSRTRFRLHVSRLVGARVVRACEDCQMIFLEDLSTGFSEGSNNSLARLFSAGQLKNSIVMAAEKVGKAVVFVCKDGTSIKDPVFGLHGLRPSKKTPGIPPDKKRLYVRRDGKIGYINADQAASLNVLLVGLSHSVAKYKFFVKDGKLQDDEEVAAQQGKKPRKIGVRLERLLKMNFDTPARLFFSLEKDQVVPCYEATETPYTGWVYVYHDRLLTTRQRDQQVNQLAQEVKDLLRDGVKIPKWDVIPDCDNSCLGFSPCLVLDDETAAVSVV

SEQ ID NO: 2 - protein, 867 aa, synthetic construct
MELKTTFCQSLKIKILPESHKLYYDELQADLKKMRIFFATHSGLNYSYKEDENDSEWKIASSKKSTLSPEAQRLIDIIAKGGIGAEHAYGIFYKDKKVSDKIRRDNSNCIFAMLLPNLVNFEEEYKKRKSKLQLTKEEWEEYHKRCLAGESVEVLWSEEIAPKIGPNEAARLASLAYHQFPDSRHPADITHCRALMSMVADSFCSWVECHKLHREETEKLKTKLQEKIESCKEAYNLLLAYSEDLYSRNYGLSARVLKALIDNKSRCCFDIAFQIAEKYPALMKIDEKTLFKAYNALKLHWKIKRRKPFISLPKIERDYQVPFGLTGSRGKKFDVYVENKNIVVEIDGQKVETFSSHYFSDMQISKIYGEKKNLKGFKLKFRHKLKDKKKEVYGEWIEAELKEIKIKKDMETGDFYLYLPYTTTHNEKNLLLEKFFSYADPLKETIFKNGKTELPNEFLGFGFDLNLSDPIAMAVAEFVRDSSDGEIGALDYGHGKLLDASVLVCNSSLSKRINDLVGDNRKLIQAIRSYKNSLVTKEMDEESGEWLKKALGNAKYGNHRHQLQLVMSKLNKKSKKYWQECRKNGHNDLSENIALLKLLDVQFSLHKSYNNIHIFDYKKQIHSHKTDSKRENFREFVTKQFAATIVKHCKEVMAKYGREYAVVFLEDLEMYFDADADNNSLIRLFAPGQLKKYIASALAKSKIGYVFIPPAGTSKTDPLTSKIGFRATGKYYKQLGYLLNKSDLYVERNGVIGKINSDVAAAINILLKGVNHSIVPYRFLNKHAGSEQKRLKRFEGEIGCDLKKMPKTRLYYLDGKIITEEEKNSLEEALLAEIKHFLLSKANIPEFDAIPGSNGTCKAFSVPRCLK

SEQ ID NO: 3 - protein, 905 aa, synthetic construct
NLAILEMREEVADYYADLQADYRHYLPLMILWGQNGFMEPDETGLNFSAVLPSTAKTRCENRYGAQIAADLKLIADDLPSSLYAIFTNDYQPLTGVKKVGDRWKFVTSLNTVSDEEFAKKVKPLLRDYKGVTEEQWRRFREERVAGKTADQVWDDLDSILRNPTLAALKKIHDKHMFRVKRPESKLYCDALAHMLSSSYDSWKKINEARQLELQAMRDEQQGLLANKTTNSLYNLVCEFADELESQNYGLTRRIFFIARKEWEDEKSHYLPVIKLLVTEKYLPLLQADNKTVSPAVKAWDICSTLKQRSPYIRFVPPTKDYPIPFGQTGRGHKFTLKEEAGKVCFLVPGLTKDEMPLSGSHYFSSLRIDKVGTDFKISFRHKTKIRSKKSARVTVKQPRINGTVTEVGILRRNGKFFLRVAYKIAYPQHNIDLKSYFASAAPSTQKLELLPAQMRVAGVDLNISDPIVVSKADIFKGEDGGPLSVLDYGSGKVVGEPVIICKDTSRAGKISSLATKCRTLRDVIRNFRHSLHQGTPLPSKSLEFLQEVPVAQEDLKSPSPRYLIQTWIKYIRTEMKRLHYKVWLDGYCHTSEAMRMLDLVDQVAFLMKSYENIHVKPGHKIPKKTTASLKIRETSRTRFRLHVSRLVGARVVRACEDCQMIFLEDLSTGFSEGSNNSLARLFSAGQLKNSIVMAAEKVGKAVVFVCKDGTSIKDPVFGLHGLRPSKKTPGIPPDKKRLYVRRDGKIGYINADQAASLNVLLVGLSHSVAKYKFFVKDGKLQDDEEVAAQQGKKPRKIGVRLERLLKMNFDTPARLFFSLEKDQVVPCYEATETPYTGWVYVYHDRLLTTRQRDQQVNQLAQEVKDLLRDGVKIPKWDVIPDCDNSCLGFSPCLVLDDETAAVSVV

SEQ ID NO: 4 - DNA, 2811 bp, synthetic construct
  source 1..2811: mol_type = other DNA
atgtccaaagacggtgacaagatgaagcacagcaaacaagaagtctctgaggacaaaggggtcaagaaagacccgtctgtcttccgctgtcagaacctcgcgattctggagatgcgtgaagaagtcgctgattactacgccgaccttcaggccgactaccgtcactacttgcccctgatgatcctctggggccagaacgggttcatggagccagacgaaactggtctgaacttcagtgccgtcctgccatcgaccgcaaaaacacggtgcgagaaccgctacggtgcgcagattgcggctgatctcaagttgatcgccgacgatctcccgagcagtctctatgccatcttcaccaacgactaccagcccctcaccggcgtgaagaaggtcggcgacagatggaagttcgtcacgtccctcaacactgtcagcgacgaagagttcgccaagaaggtgaagccgctcctgcgtgactacaaaggcgttaccgaagagcagtggcgtcgatttcgtgaagagcgtgtcgccggcaagacagccgatcaggtctgggacgaccttgacagcattctgcgaaacccgacgctcgcggccctgaagaagatccacgacaagcacatgttccgggtcaagcgacccgagagcaaactctactgtgacgccctggctcacatgctgagttccagctacgacagttggaagaaaatcaacgaggcccgccaactcgaactccaagccatgcgtgacgagcagcagggcttgctcgccaacaagaccacgaacagtctgtacaatctggtgtgcgagttcgccgacgaactggagtcgcagaactacggtctgactcggcggatcttcttcatcgcccgcaaagagtgggaggacgaaaagtcccattacctcccggtcatcaagctcctcgtcacggagaagtatctgcccctgctccaggcggataacaagactgtcagccccgccgtcaaggcgtgggacatctgctctactctgaagcagaggtcgccgtacatccgcttcgtgcctccgaccaaggactatccgatccccttcggccagacgggtcgcggacacaagttcaccttgaaagaggaagccggaaaggtctgtttcctcgtacccggcctgaccaaggacgagatgcctctgtccggctctcactacttctcaagcctccgtatcgacaaggtcggcacagacttcaagatctcgttccgtcacaagaccaagattcgcagcaaaaagtctgcgcgagtcaccgtcaagcagcccaggatcaacggcaccgtcactgaggtcggcatcctacgcaggaacggcaagttcttcctgcgtgtcgcttacaagatcgcctacccacagcacaacatcgacctgaagagctacttcgcctcggcagccccctccacgcaaaagctggagcttctgccggctcaaatgagagtcgcgggcgtcgatctgaacatcagcgacccgattgtcgtctccaaggccgacatcttcaagggcgaagacggcgggccgctgtctgtgctggactacggctctggcaaggtggtaggtgagcctgtgatcatctgcaaggacaccagcagggccgggaagatcagttccctcgccacgaaatgccggacgctacgcgacgtgatccgcaactttcgtcattcactgcatcagggtacgcccttgcccagcaagagtcttgaatttctccaagaagttcccgtggcgcaggaggacttgaagtcgcccagccctcgctatctgatccagacgtggatcaagtacatcaggaccgagatgaagcgacttcactacaaggtctggctcgacgggtactgccacactagcgaagccatgcggatgctcgatctggtcgatcaggtcgcgttcctaatgaagtcgtatgagaacatccacgtcaaaccgggtcacaagatcccgaagaagaccactgcgtccctgaagattcgcgagacgagccgaacgcggttcaggctgcatgtctcccgacttgtcggtgcgcgagtggttcgtgcgtgcgaggactgccagatgatcttcttggaagatctctcgacaggtttctccgaaggcagcaataactcgttggcccggctgttttcggctggccaactgaagaactccatcgtcatggcggcagagaaggtcggcaaagccgtagtcttcgtgtgcaaggacggtacgtctatcaaagatcctgttttcggtctgcacggcctgcggccttccaagaagaccccaggcattccgccggacaagaagcgtctctacgtccgtcgcgacggcaaaatcggctacatcaacgccgatcaggcggcttcgctgaatgtgctgctggtggggctgtcgcactccgtcgccaagtacaagttctttgtcaaggacgggaagctacaagacgacgaggaggtggctgcccagcaaggcaagaagccgcgtaagatcggtgttcgtctggaaagactcctcaagatgaacttcgacacccctgccagacttttcttctcgctggagaaagatcaggtggtgccgtgctacgaggccacggaaacgccttacaccggctgggtctacgtctaccacgaccgactgctgaccacccggcaacgtgatcagcaagtcaatcaactcgcacaggaagtcaaggatctcctgcgggatggtgtcaaaattcctaaatgggacgtaatcccagactgcgacaacagttgcctcggcttctctccgtgtctggtgcttgacgacgaaaccgccgctgtttctgtggtttag

SEQ ID NO: 5 - DNA, 2604 bp, synthetic construct
  source 1..2604: mol_type = other DNA
atggaattgaagacaactttttgtcagtcactaaaaattaagatcctccccgaaagccataagttgtattacgatgagctgcaagccgacctaaagaagatgagaatcttcttcgcaacgcactctgggctcaactacagctacaaggaagatgaaaacgattcggaatggaagatagcgtcgtcaaaaaagagcacactctcacctgaagcccaaaggcttattgacatcatcgccaaaggcggtataggagcagagcacgcctacggaatattttacaaggacaaaaaggtgtcggacaagatccggcgagataattcaaactgcatcttcgccatgctattacctaacctcgtcaatttcgaggaagaatacaaaaagaggaaaagtaagctccagctcaccaaggaagagtgggaggagtaccataaacgatgtcttgcaggcgagagcgtggaagtcctgtggtccgaggagattgctccaaaaattgggcctaacgaagcggcgagactggcatcactcgcgtatcaccagttcccagattctcgtcatccggcggacattacccactgcagggcgctcatgtctatggtagcagactctttttgttcctgggtcgagtgccacaagctgcaccgtgaggaaacagagaaactcaagacgaaattacaggagaagatagagagctgcaaggaggcatacaatctgctactcgcttattccgaagacctgtacagtaggaattatggtctgagtgccagggttttgaaggcacttattgacaacaaatcccggtgctgcttcgacatagcctttcagatcgcggagaaatatcctgcactgatgaagattgatgaaaagaccctgttcaaggcctacaacgccctaaagctccactggaaaataaaacgacgcaagccgttcatctcgctgcccaagatcgagagggactaccaggtgccctttggactcacaggaagcagaggcaagaagttcgacgtctatgtggaaaacaagaatattgtggtcgagatcgacggccagaaagtcgaaaccttcagctctcactatttctctgatatgcagatctccaagatttacggtgaaaagaaaaatttgaagggattcaagttgaaattccgccacaagctgaaggacaagaagaaggaggtttatggagagtggattgaggcggagctgaaggaaatcaagattaagaaagacatggagacaggcgacttctacctgtatctcccatacactacaacgcataatgagaaaaacttattgctcgagaagttcttttcctacgccgatccacttaaggagactatttttaagaacggaaagacggagcttccaaatgaatttctaggcttcggcttcgatttgaacctttccgacccgattgcgatggcggttgccgagtttgttcgggactcatcagatggtgagattggcgccctcgactatggtcacggtaagctcctcgacgctagtgttttggtctgcaactcatcactatcaaagcgcatcaatgacctggttggagacaatagaaagctgatccaagctattagatcgtacaagaactcgctagtaactaaggaaatggacgaggagagtggcgagtggcttaaaaaagctctaggcaatgcaaagtacggcaaccataggcatcagcttcaactggtcatgtctaagctgaacaaaaaaagcaaaaagtactggcaggaatgtcgcaagaacggccacaacgatctttccgaaaatattgcactattaaagttgttggacgtgcagttctctttgcataaaagctataacaacatacacatctttgattacaaaaaacaaattcacagccataagactgactccaaaagggagaatttccgtgaatttgtgacgaagcaatttgcggctaccatagtaaagcattgcaaggaggtgatggctaaatatggcagagagtacgccgttgtctttttggaggacctggagatgtactttgatgctgatgctgataacaattctctgatccggctttttgcacccggccaattgaagaagtacatcgcctccgctctggctaagagcaagataggttatgtgttcatccctcctgctgggacaagtaagaccgacccgttaacttcgaaaattggattcagggctaccggaaaatactacaagcagctgggctatctgcttaataagtccgacctctacgtggagcgcaacggggttatcgggaagatcaacagtgatgtagctgcggcgataaatattctcttaaagggcgtgaaccactcaatcgtgccgtaccgcttcctgaataaacatgctgggtctgaacagaagcgcctcaaacgttttgaaggggagatcggttgtgatctgaaaaagatgccaaagacacgcctctactatcttgacggaaaaatcatcactgaagaggagaagaacagcctggaggaagcgctactcgctgagatcaagcacttccttctgtcgaaggccaacattccggagtttgatgcaataccggggagcaatggcacctgcaaagccttctccgttccacgctgccttaagtga

SEQ ID NO: 6 - DNA, 2718 bp, synthetic construct
  source 1..2718: mol_type = other DNA
aacctcgcgattctggagatgcgtgaagaagtcgctgattactacgccgaccttcaggccgactaccgtcactacttgcccctgatgatcctctggggccagaacgggttcatggagccagacgaaactggtctgaacttcagtgccgtcctgccatcgaccgcaaaaacacggtgcgagaaccgctacggtgcgcagattgcggctgatctcaagttgatcgccgacgatctcccgagcagtctctatgccatcttcaccaacgactaccagcccctcaccggcgtgaagaaggtcggcgacagatggaagttcgtcacgtccctcaacactgtcagcgacgaagagttcgccaagaaggtgaagccgctcctgcgtgactacaaaggcgttaccgaagagcagtggcgtcgatttcgtgaagagcgtgtcgccggcaagacagccgatcaggtctgggacgaccttgacagcattctgcgaaacccgacgctcgcggccctgaagaagatccacgacaagcacatgttccgggtcaagcgacccgagagcaaactctactgtgacgccctggctcacatgctgagttccagctacgacagttggaagaaaatcaacgaggcccgccaactcgaactccaagccatgcgtgacgagcagcagggcttgctcgccaacaagaccacgaacagtctgtacaatctggtgtgcgagttcgccgacgaactggagtcgcagaactacggtctgactcggcggatcttcttcatcgcccgcaaagagtgggaggacgaaaagtcccattacctcccggtcatcaagctcctcgtcacggagaagtatctgcccctgctccaggcggataacaagactgtcagccccgccgtcaaggcgtgggacatctgctctactctgaagcagaggtcgccgtacatccgcttcgtgcctccgaccaaggactatccgatccccttcggccagacgggtcgcggacacaagttcaccttgaaagaggaagccggaaaggtctgtttcctcgtacccggcctgaccaaggacgagatgcctctgtccggctctcactacttctcaagcctccgtatcgacaaggtcggcacagacttcaagatctcgttccgtcacaagaccaagattcgcagcaaaaagtctgcgcgagtcaccgtcaagcagcccaggatcaacggcaccgtcactgaggtcggcatcctacgcaggaacggcaagttcttcctgcgtgtcgcttacaagatcgcctacccacagcacaacatcgacctgaagagctacttcgcctcggcagccccctccacgcaaaagctggagcttctgccggctcaaatgagagtcgcgggcgtcgatctgaacatcagcgacccgattgtcgtctccaaggccgacatcttcaagggcgaagacggcgggccgctgtctgtgctggactacggctctggcaaggtggtaggtgagcctgtgatcatctgcaaggacaccagcagggccgggaagatcagttccctcgccacgaaatgccggacgctacgcgacgtgatccgcaactttcgtcattcactgcatcagggtacgcccttgcccagcaagagtcttgaatttctccaagaagttcccgtggcgcaggaggacttgaagtcgcccagccctcgctatctgatccagacgtggatcaagtacatcaggaccgagatgaagcgacttcactacaaggtctggctcgacgggtactgccacactagcgaagccatgcggatgctcgatctggtcgatcaggtcgcgttcctaatgaagtcgtatgagaacatccacgtcaaaccgggtcacaagatcccgaagaagaccactgcgtccctgaagattcgcgagacgagccgaacgcggttcaggctgcatgtctcccgacttgtcggtgcgcgagtggttcgtgcgtgcgaggactgccagatgatcttcttggaagatctctcgacaggtttctccgaaggcagcaataactcgttggcccggctgttttcggctggccaactgaagaactccatcgtcatggcggcagagaaggtcggcaaagccgtagtcttcgtgtgcaaggacggtacgtctatcaaagatcctgttttcggtctgcacggcctgcggccttccaagaagaccccaggcattccgccggacaagaagcgtctctacgtccgtcgcgacggcaaaatcggctacatcaacgccgatcaggcggcttcgctgaatgtgctgctggtggggctgtcgcactccgtcgccaagtacaagttctttgtcaaggacgggaagctacaagacgacgaggaggtggctgcccagcaaggcaagaagccgcgtaagatcggtgttcgtctggaaagactcctcaagatgaacttcgacacccctgccagacttttcttctcgctggagaaagatcaggtggtgccgtgctacgaggccacggaaacgccttacaccggctgggtctacgtctaccacgaccgactgctgaccacccggcaacgtgatcagcaagtcaatcaactcgcacaggaagtcaaggatctcctgcgggatggtgtcaaaattcctaaatgggacgtaatcccagactgcgacaacagttgcctcggcttctctccgtgtctggtgcttgacgacgaaaccgccgctgtttctgtggtttag

SEQ ID NO: 7 - RNA, 36 nt, synthetic construct
  source 1..36: mol_type = other RNA
gtgctgacgaccagcactagatggtcgttcaggcac

SEQ ID NO: 8 - RNA, 36 nt, synthetic construct
  source 1..36: mol_type = other RNA
gtgctgaacagggtcgctaggcgttgttcaaggcac

SEQ ID NO: 9 - RNA, 34 nt, synthetic construct
  source 1..34: mol_type = other RNA
gctgacgaccagcactagatggtcgttcaggcac

SEQ ID NO: 10 - RNA, 32 nt, synthetic construct
  source 1..32: mol_type = other RNA
tgacgaccagcactagatggtcgttcaggcac

SEQ ID NO: 11 - RNA, 30 nt, synthetic construct
  source 1..30: mol_type = other RNA
acgaccagcactagatggtcgttcaggcac

SEQ ID NO: 12 - RNA, 28 nt, synthetic construct
  source 1..28: mol_type = other RNA
gaccagcactagatggtcgttcaggcac

SEQ ID NO: 13 - RNA, 26 nt, synthetic construct
  source 1..26: mol_type = other RNA
ccagcactagatggtcgttcaggcac

SEQ ID NO: 14 - RNA, 24 nt, synthetic construct
  source 1..24: mol_type = other RNA
agcactagatggtcgttcaggcac

SEQ ID NO: 15 - RNA, 22 nt, synthetic construct
  source 1..22: mol_type = other RNA
cactagatggtcgttcaggcac

SEQ ID NO: 16 - RNA, 20 nt, synthetic construct
  source 1..20: mol_type = other RNA
ctagatggtcgttcaggcac

SEQ ID NO: 17 - DNA, 36 bp, synthetic construct
  source 1..36: mol_type = other DNA
gtgctgacgaccagcactagatggtcgttcaggcac

SEQ ID NO: 18 - DNA, 37 bp, synthetic construct
  source 1..37: mol_type = other DNA
gtgctgaacagggtcgctaggcgttgttcaaggcacg

SEQ ID NO: 19 - DNA, 34 bp, synthetic construct
  source 1..34: mol_type = other DNA
gctgacgaccagcactagatggtcgttcaggcac

SEQ ID NO: 20 - DNA, 32 bp, synthetic construct
  source 1..32: mol_type = other DNA
tgacgaccagcactagatggtcgttcaggcac

SEQ ID NO: 21 - DNA, 30 bp, synthetic construct
  source 1..30: mol_type = other DNA
acgaccagcactagatggtcgttcaggcac

SEQ ID NO: 22 - DNA, 28 bp, synthetic construct
  source 1..28: mol_type = other DNA
gaccagcactagatggtcgttcaggcac

SEQ ID NO: 23 - protein, 26 aa, synthetic construct
CCAGCACTAGATGGTCGTTCAGGCAC

SEQ ID NO: 24 - DNA, 24 bp, synthetic construct
  source 1..24: mol_type = other DNA
agcactagatggtcgttcaggcac

SEQ ID NO: 25 - DNA, 22 bp, synthetic construct
  source 1..22: mol_type = other DNA
cactagatggtcgttcaggcac

SEQ ID NO: 26 - DNA, 20 bp, synthetic construct
  source 1..20: mol_type = other DNA
ctagatggtcgttcaggcac

SEQ ID NO: 27 - protein, 11 aa, synthetic construct
SRADPKKKRKV

SEQ ID NO: 28 - protein, 979 aa, synthetic construct
MGPKKKRKVMDYKDHDGDYKDHDIDYKDDDDKMSKDGDKMKHSKQEVSEDKGVKKDPSVFRCQNLAILEMREEVADYYADLQADYRHYLPLMILWGQNGFMEPDETGLNFSAVLPSTAKTRCENRYGAQIAADLKLIADDLPSSLYAIFTNDYQPLTGVKKVGDRWKFVTSLNTVSDEEFAKKVKPLLRDYKGVTEEQWRRFREERVAGKTADQVWDDLDSILRNPTLAALKKIHDKHMFRVKRPESKLYCDALAHMLSSSYDSWKKINEARQLELQAMRDEQQGLLANKTTNSLYNLVCEFADELESQNYGLTRRIFFIARKEWEDEKSHYLPVIKLLVTEKYLPLLQADNKTVSPAVKAWDICSTLKQRSPYIRFVPPTKDYPIPFGQTGRGHKFTLKEEAGKVCFLVPGLTKDEMPLSGSHYFSSLRIDKVGTDFKISFRHKTKIRSKKSARVTVKQPRINGTVTEVGILRRNGKFFLRVAYKIAYPQHNIDLKSYFASAAPSTQKLELLPAQMRVAGVDLNISDPIVVSKADIFKGEDGGPLSVLDYGSGKVVGEPVIICKDTSRAGKISSLATKCRTLRDVIRNFRHSLHQGTPLPSKSLEFLQEVPVAQEDLKSPSPRYLIQTWIKYIRTEMKRLHYKVWLDGYCHTSEAMRMLDLVDQVAFLMKSYENIHVKPGHKIPKKTTASLKIRETSRTRFRLHVSRLVGARVVRACEDCQMIFLEDLSTGFSEGSNNSLARLFSAGQLKNSIVMAAEKVGKAVVFVCKDGTSIKDPVFGLHGLRPSKKTPGIPPDKKRLYVRRDGKIGYINADQAASLNVLLVGLSHSVAKYKFFVKDGKLQDDEEVAAQQGKKPRKIGVRLERLLKMNFDTPARLFFSLEKDQVVPCYEATETPYTGWVYVYHDRLLTTRQRDQQVNQLAQEVKDLLRDGVKIPKWDVIPDCDNSCLGFSPCLVLDDETAAVSVVSRADPKKKRKV

SEQ ID NO: 29 - protein, 910 aa, synthetic construct
MGPKKKRKVMDYKDHDGDYKDHDIDYKDDDDKMELKTTFCQSLKIKILPESHKLYYDELQADLKKMRIFFATHSGLNYSYKEDENDSEWKIASSKKSTLSPEAQRLIDIIAKGGIGAEHAYGIFYKDKKVSDKIRRDNSNCIFAMLLPNLVNFEEEYKKRKSKLQLTKEEWEEYHKRCLAGESVEVLWSEEIAPKIGPNEAARLASLAYHQFPDSRHPADITHCRALMSMVADSFCSWVECHKLHREETEKLKTKLQEKIESCKEAYNLLLAYSEDLYSRNYGLSARVLKALIDNKSRCCFDIAFQIAEKYPALMKIDEKTLFKAYNALKLHWKIKRRKPFISLPKIERDYQVPFGLTGSRGKKFDVYVENKNIVVEIDGQKVETFSSHYFSDMQISKIYGEKKNLKGFKLKFRHKLKDKKKEVYGEWIEAELKEIKIKKDMETGDFYLYLPYTTTHNEKNLLLEKFFSYADPLKETIFKNGKTELPNEFLGFGFDLNLSDPIAMAVAEFVRDSSDGEIGALDYGHGKLLDASVLVCNSSLSKRINDLVGDNRKLIQAIRSYKNSLVTKEMDEESGEWLKKALGNAKYGNHRHQLQLVMSKLNKKSKKYWQECRKNGHNDLSENIALLKLLDVQFSLHKSYNNIHIFDYKKQIHSHKTDSKRENFREFVTKQFAATIVKHCKEVMAKYGREYAVVFLEDLEMYFDADADNNSLIRLFAPGQLKKYIASALAKSKIGYVFIPPAGTSKTDPLTSKIGFRATGKYYKQLGYLLNKSDLYVERNGVIGKINSDVAAAINILLKGVNHSIVPYRFLNKHAGSEQKRLKRFEGEIGCDLKKMPKTRLYYLDGKIITEEEKNSLEEALLAEIKHFLLSKANIPEFDAIPGSNGTCKAFSVPRCLKSRADPKKKRKV

SEQ ID NO: 30 - protein, 948 aa, synthetic construct
MGPKKKRKVMDYKDHDGDYKDHDIDYKDDDDKNLAILEMREEVADYYADLQADYRHYLPLMILWGQNGFMEPDETGLNFSAVLPSTAKTRCENRYGAQIAADLKLIADDLPSSLYAIFTNDYQPLTGVKKVGDRWKFVTSLNTVSDEEFAKKVKPLLRDYKGVTEEQWRRFREERVAGKTADQVWDDLDSILRNPTLAALKKIHDKHMFRVKRPESKLYCDALAHMLSSSYDSWKKINEARQLELQAMRDEQQGLLANKTTNSLYNLVCEFADELESQNYGLTRRIFFIARKEWEDEKSHYLPVIKLLVTEKYLPLLQADNKTVSPAVKAWDICSTLKQRSPYIRFVPPTKDYPIPFGQTGRGHKFTLKEEAGKVCFLVPGLTKDEMPLSGSHYFSSLRIDKVGTDFKISFRHKTKIRSKKSARVTVKQPRINGTVTEVGILRRNGKFFLRVAYKIAYPQHNIDLKSYFASAAPSTQKLELLPAQMRVAGVDLNISDPIVVSKADIFKGEDGGPLSVLDYGSGKVVGEPVIICKDTSRAGKISSLATKCRTLRDVIRNFRHSLHQGTPLPSKSLEFLQEVPVAQEDLKSPSPRYLIQTWIKYIRTEMKRLHYKVWLDGYCHTSEAMRMLDLVDQVAFLMKSYENIHVKPGHKIPKKTTASLKIRETSRTRFRLHVSRLVGARVVRACEDCQMIFLEDLSTGFSEGSNNSLARLFSAGQLKNSIVMAAEKVGKAVVFVCKDGTSIKDPVFGLHGLRPSKKTPGIPPDKKRLYVRRDGKIGYINADQAASLNVLLVGLSHSVAKYKFFVKDGKLQDDEEVAAQQGKKPRKIGVRLERLLKMNFDTPARLFFSLEKDQVVPCYEATETPYTGWVYVYHDRLLTTRQRDQQVNQLAQEVKDLLRDGVKIPKWDVIPDCDNSCLGFSPCLVLDDETAAVSVVSRADPKKKRKV

SEQ ID NO: 31 - DNA, 2967 bp, synthetic construct
  source 1..2967: mol_type = other DNA
atgggaccaaagaagaagagaaaggttatggattacaaggaccacgacggagactataaagatcatgacatcgattataaggatgatgatgacaagatgtctaaagacggggacaaaatgaaacactccaagcaagaagtttccgaggacaaaggcgtcaagaaagatccgtccgtgtttcgatgccagaacctcgccatactcgagatgagggaggaggtcgctgactattacgcagaccttcaggctgactaccggcactacctgcctctgatgattctgtgggggcaaaatggctttatggagccagatgaaacgggcctcaacttcagcgccgtcctgccgtcgaccgcgaaaacacgctgtgagaataggtatggtgcccagattgcagccgacctgaaactcatcgcagatgatctgccgtctagcctgtatgctatattcacgaatgattaccagcctttaactggcgtgaagaaggttggtgaccgttggaagtttgtgacttcattgaacaccgtgtcggatgaggaatttgcaaagaaagtcaaacccctccttcgcgactacaaaggtgtcacagaggagcagtggcgtcgtttccgtgaggaacgagttgctgggaaaacagccgaccaggtttgggacgatctcgattccatcttgaggaacccgacgcttgccgccctcaaaaagattcatgacaagcacatgttcagagttaagaggccggagtcaaagctgtattgtgatgcattggcgcatatgctctcatcttcatatgattcatggaaaaagatcaatgaagcacgtcagttggaactacaggccatgagagacgaacaacaaggcctgctggcaaataaaactactaactctctttacaatctggtctgtgagttcgcggatgagcttgaatcacaaaactacggcctgacccgaaggatattcttcatcgccaggaaagagtgggaggatgaaaagtcgcactaccttcctgtgatcaaactcttggtgaccgagaagtatcttccacttctgcaggcggataacaaaacagtttctccggcggttaaggcgtgggatatttgcagcacgttgaagcagcgcagtccatatatacggttcgttcctcccacaaaagactatccgattccatttggccagacaggtaggggacacaagtttacactcaaggaggaggccgggaaggtttgcttcctggttccaggactcaccaaggatgaaatgccactctcgggctcccactatttctctagtctgcgaattgacaaggtcggcacagacttcaagatcagcttccgccataagacgaagattcgaagcaagaagtcagctagagtgaccgtaaaacaacctcggatcaacggcaccgttacggaggtgggtatcctgcgtcgcaacggcaagtttttcttacgggttgcgtacaagattgcttacccgcaacacaacattgatcttaagagctacttcgccagcgccgccccttccactcagaagctcgagctgctccctgctcagatgagggtggcgggagtcgatttgaacatttccgatcccatcgtcgtatccaaagctgatatttttaagggagaggatggagggccattatcggtcttggactacggatctggcaaggtagtgggcgagcccgtcatcatctgtaaggatacaagtcgcgcaggtaaaatctcctcgctcgcgactaagtgccgcaccttaagagacgtcattcggaatttccgccactctctccatcagggaacccccctacccagtaagagcctggagtttctgcaggaggtgccagtggcccaggaggacctaaaatcgccatcccccaggtatctcatacagacctggataaagtatatcagaacagagatgaagcgcttgcattataaagtgtggttggacggctactgccacacatctgaagctatgcgtatgcttgatctagttgatcaggtcgccttcctcatgaagtcatacgaaaacatacatgtgaagccgggccataaaattcccaagaaaactaccgcgagtctcaagattagggaaacatccaggacgagatttagactgcatgtatccaggttggtcggggccagggtggtccgcgcctgtgaagattgtcagatgatcttcctggaagacttgtcgaccggcttctcagaaggtagcaacaatagcctcgcgcggctgtttagcgccggccagctgaagaatagtatagtgatggcggcggagaaggtgggtaaagctgttgtatttgtctgcaaggatggaactagcataaaggaccctgtcttcggtttacacggccttcggccgtcgaaaaagaccccaggcatccctccggataaaaaaagactttatgtgagacgggatggtaagattgggtacatcaatgctgatcaagctgcaagtctgaatgtactattggtaggtctgagccatagtgtggccaagtacaagttcttcgtcaaagatggtaagttgcaggatgacgaagaggtggcggcacaacaaggaaagaagcctaggaagattggcgttcgcctggaaagactactaaagatgaattttgataccccggcccgccttttttttagcctggagaaggaccaggtggttccgtgctacgaggcgactgagacgccttacacgggttgggtgtacgtctaccatgacaggcttctcacaacacgccagagggaccaacaggtgaatcaacttgcccaagaggtgaaagacttacttagggacggggtgaagatccctaaatgggacgtgatcccagactgcgacaacagttgcctcgggttctctccctgcctggtgcttgacgacgagactgctgctgtctcagttgttggatctgggagcaagcggcccgcagcaacgaaaaaggcagggcaagcaaaaaagaagaaatga

SEQ ID NO: 32 - DNA, 2760 bp, synthetic construct
  source 1..2760: mol_type = other DNA
atgggtccgaaaaagaagcggaaagttatggattataaggatcatgatggcgactataaggatcatgacatagattataaagatgatgacgacaaaatggaattgaagacaactttttgtcagtcactaaaaattaagatcctccccgaaagccataagttgtattacgatgagctgcaagccgacctaaagaagatgagaatcttcttcgcaacgcactctgggctcaactacagctacaaggaagatgaaaacgattcggaatggaagatagcgtcgtcaaaaaagagcacactctcacctgaagcccaaaggcttattgacatcatcgccaaaggcggtataggagcagagcacgcctacggaatattttacaaggacaaaaaggtgtcggacaagatccggcgagataattcaaactgcatcttcgccatgctattacctaacctcgtcaatttcgaggaagaatacaaaaagaggaaaagtaagctccagctcaccaaggaagagtgggaggagtaccataaacgatgtcttgcaggcgagagcgtggaagtcctgtggtccgaggagattgctccaaaaattgggcctaacgaagcggcgagactggcatcactcgcgtatcaccagttcccagattctcgtcatccggcggacattacccactgcagggcgctcatgtctatggtagcagactctttttgttcctgggtcgagtgccacaagctgcaccgtgaggaaacagagaaactcaagacgaaattacaggagaagatagagagctgcaaggaggcatacaatctgctactcgcttattccgaagacctgtacagtaggaattatggtctgagtgccagggttttgaaggcacttattgacaacaaatcccggtgctgcttcgacatagcctttcagatcgcggagaaatatcctgcactgatgaagattgatgaaaagaccctgttcaaggcctacaacgccctaaagctccactggaaaataaaacgacgcaagccgttcatctcgctgcccaagatcgagagggactaccaggtgccctttggactcacaggaagcagaggcaagaagttcgacgtctatgtggaaaacaagaatattgtggtcgagatcgacggccagaaagtcgaaaccttcagctctcactatttctctgatatgcagatctccaagatttacggtgaaaagaaaaatttgaagggattcaagttgaaattccgccacaagctgaaggacaagaagaaggaggtttatggagagtggattgaggcggagctgaaggaaatcaagattaagaaagacatggagacaggcgacttctacctgtatctcccatacactacaacgcataatgagaaaaacttattgctcgagaagttcttttcctacgccgatccacttaaggagactatttttaagaacggaaagacggagcttccaaatgaatttctaggcttcggcttcgatttgaacctttccgacccgattgcgatggcggttgccgagtttgttcgggactcatcagatggtgagattggcgccctcgactatggtcacggtaagctcctcgacgctagtgttttggtctgcaactcatcactatcaaagcgcatcaatgacctggttggagacaatagaaagctgatccaagctattagatcgtacaagaactcgctagtaactaaggaaatggacgaggagagtggcgagtggcttaaaaaagctctaggcaatgcaaagtacggcaaccataggcatcagcttcaactggtcatgtctaagctgaacaaaaaaagcaaaaagtactggcaggaatgtcgcaagaacggccacaacgatctttccgaaaatattgcactattaaagttgttggacgtgcagttctctttgcataaaagctataacaacatacacatctttgattacaaaaaacaaattcacagccataagactgactccaaaagggagaatttccgtgaatttgtgacgaagcaatttgcggctaccatagtaaagcattgcaaggaggtgatggctaaatatggcagagagtacgccgttgtctttttggaggacctggagatgtactttgatgctgatgctgataacaattctctgatccggctttttgcacccggccaattgaagaagtacatcgcctccgctctggctaagagcaagataggttatgtgttcatccctcctgctgggacaagtaagaccgacccgttaacttcgaaaattggattcagggctaccggaaaatactacaagcagctgggctatctgcttaataagtccgacctctacgtggagcgcaacggggttatcgggaagatcaacagtgatgtagctgcggcgataaatattctcttaaagggcgtgaaccactcaatcgtgccgtaccgcttcctgaataaacatgctgggtctgaacagaagcgcctcaaacgttttgaaggggagatcggttgtgatctgaaaaagatgccaaagacacgcctctactatcttgacggaaaaatcatcactgaagaggagaagaacagcctggaggaagcgctactcgctgagatcaagcacttccttctgtcgaaggccaacattccggagtttgatgcaataccggggagcaatggcacctgcaaagccttctccgttccacgctgccttaagggcagcgggagcaagcggcccgccgcgaccaaaaaagcggggcaagccaagaagaaaaagtga

SEQ ID NO: 33 - DNA, 2874 bp, synthetic construct
  source 1..2874: mol_type = other DNA
atgggaccaaagaagaagagaaaggttatggattacaaggaccacgacggagactataaagatcatgacatcgattataaggatgatgatgacaagaacctcgccatactcgagatgagggaggaggtcgctgactattacgcagaccttcaggctgactaccggcactacctgcctctgatgattctgtgggggcaaaatggctttatggagccagatgaaacgggcctcaacttcagcgccgtcctgccgtcgaccgcgaaaacacgctgtgagaataggtatggtgcccagattgcagccgacctgaaactcatcgcagatgatctgccgtctagcctgtatgctatattcacgaatgattaccagcctttaactggcgtgaagaaggttggtgaccgttggaagtttgtgacttcattgaacaccgtgtcggatgaggaatttgcaaagaaagtcaaacccctccttcgcgactacaaaggtgtcacagaggagcagtggcgtcgtttccgtgaggaacgagttgctgggaaaacagccgaccaggtttgggacgatctcgattccatcttgaggaacccgacgcttgccgccctcaaaaagattcatgacaagcacatgttcagagttaagaggccggagtcaaagctgtattgtgatgcattggcgcatatgctctcatcttcatatgattcatggaaaaagatcaatgaagcacgtcagttggaactacaggccatgagagacgaacaacaaggcctgctggcaaataaaactactaactctctttacaatctggtctgtgagttcgcggatgagcttgaatcacaaaactacggcctgacccgaaggatattcttcatcgccaggaaagagtgggaggatgaaaagtcgcactaccttcctgtgatcaaactcttggtgaccgagaagtatcttccacttctgcaggcggataacaaaacagtttctccggcggttaaggcgtgggatatttgcagcacgttgaagcagcgcagtccatatatacggttcgttcctcccacaaaagactatccgattccatttggccagacaggtaggggacacaagtttacactcaaggaggaggccgggaaggtttgcttcctggttccaggactcaccaaggatgaaatgccactctcgggctcccactatttctctagtctgcgaattgacaaggtcggcacagacttcaagatcagcttccgccataagacgaagattcgaagcaagaagtcagctagagtgaccgtaaaacaacctcggatcaacggcaccgttacggaggtgggtatcctgcgtcgcaacggcaagtttttcttacgggttgcgtacaagattgcttacccgcaacacaacattgatcttaagagctacttcgccagcgccgccccttccactcagaagctcgagctgctccctgctcagatgagggtggcgggagtcgatttgaacatttccgatcccatcgtcgtatccaaagctgatatttttaagggagaggatggagggccattatcggtcttggactacggatctggcaaggtagtgggcgagcccgtcatcatctgtaaggatacaagtcgcgcaggtaaaatctcctcgctcgcgactaagtgccgcaccttaagagacgtcattcggaatttccgccactctctccatcagggaacccccctacccagtaagagcctggagtttctgcaggaggtgccagtggcccaggaggacctaaaatcgccatcccccaggtatctcatacagacctggataaagtatatcagaacagagatgaagcgcttgcattataaagtgtggttggacggctactgccacacatctgaagctatgcgtatgcttgatctagttgatcaggtcgccttcctcatgaagtcatacgaaaacatacatgtgaagccgggccataaaattcccaagaaaactaccgcgagtctcaagattagggaaacatccaggacgagatttagactgcatgtatccaggttggtcggggccagggtggtccgcgcctgtgaagattgtcagatgatcttcctggaagacttgtcgaccggcttctcagaaggtagcaacaatagcctcgcgcggctgtttagcgccggccagctgaagaatagtatagtgatggcggcggagaaggtgggtaaagctgttgtatttgtctgcaaggatggaactagcataaaggaccctgtcttcggtttacacggccttcggccgtcgaaaaagaccccaggcatccctccggataaaaaaagactttatgtgagacgggatggtaagattgggtacatcaatgctgatcaagctgcaagtctgaatgtactattggtaggtctgagccatagtgtggccaagtacaagttcttcgtcaaagatggtaagttgcaggatgacgaagaggtggcggcacaacaaggaaagaagcctaggaagattggcgttcgcctggaaagactactaaagatgaattttgataccccggcccgccttttttttagcctggagaaggaccaggtggttccgtgctacgaggcgactgagacgccttacacgggttgggtgtacgtctaccatgacaggcttctcacaacacgccagagggaccaacaggtgaatcaacttgcccaagaggtgaaagacttacttagggacggggtgaagatccctaaatgggacgtgatcccagactgcgacaacagttgcctcgggttctctccctgcctggtgcttgacgacgagactgctgctgtctcagttgttggatctgggagcaagcggcccgcagcaacgaaaaaggcagggcaagcaaaaaagaagaaatga

SEQ ID NO: 34 - DNA, 35 bp, synthetic construct
  source 1..35: mol_type = other DNA
nnnnnnnnggtataacaacttcgacgagctctaca

SEQ ID NO: 35 - RNA, 27 nt, synthetic construct
  source 1..27: mol_type = other RNA
ggtataacaacttcgacgagctctaca

SEQ ID NO: 36 - RNA, 27 nt, synthetic construct
  source 1..27: mol_type = other RNA
ggtataacaacttcgacgagctctaca

SEQ ID NO: 37 - RNA, 26 nt, synthetic construct
  source 1..26: mol_type = other RNA
cggtgggctggcgctggggttcagct

SEQ ID NO: 38 - RNA, 26 nt, synthetic construct
  source 1..26: mol_type = other RNA
gagccagagaggatcctgggagggag

SEQ ID NO: 39 - RNA, 26 nt, synthetic construct
  source 1..26: mol_type = other RNA
tgactttgtcacagcccaagatagtt

SEQ ID NO: 40 - RNA, 26 nt, synthetic construct
  source 1..26: mol_type = other RNA
aaacccagacacatagcaattcagga

SEQ ID NO: 41 - RNA, 26 nt, synthetic construct
  source 1..26: mol_type = other RNA
ctgaggggctgctggtttggctggtg

SEQ ID NO: 42 - RNA, 26 nt, synthetic construct
  source 1..26: mol_type = other RNA
gagatgccagcagaagttgggcagaa

SEQ ID NO: 43 - RNA, 26 nt, synthetic construct
  source 1..26: mol_type = other RNA
gggcagagtggagatggtggggacaa

SEQ ID NO: 44 - RNA, 26 nt, synthetic construct
  source 1..26: mol_type = other RNA
actagggtgggcaaccacaaacccac

SEQ ID NO: 45 - RNA, 26 nt, synthetic construct
  source 1..26: mol_type = other RNA
tgtacagaaggctgaaaggagagaac

SEQ ID NO: 46 - RNA, 26 nt, synthetic construct
  source 1..26: mol_type = other RNA
cggtaccagtttagcacgaagctctc

SEQ ID NO: 47 - RNA, 26 nt, synthetic construct
  source 1..26: mol_type = other RNA
ccaccattgtctttcctagcggaatg

SEQ ID NO: 48 - RNA, 26 nt, synthetic construct
  source 1..26: mol_type = other RNA
aagcactgtgggtacgaaggaaatga

SEQ ID NO: 49 - RNA, 26 nt, synthetic construct
  source 1..26: mol_type = other RNA
tgtcacaaagtaaggattctgatgtg

SEQ ID NO: 50 - RNA, 26 nt, synthetic construct
  source 1..26: mol_type = other RNA
ctgttgttgaaggcgtttgcacatgc

SEQ ID NO: 51 - RNA, 26 nt, synthetic construct
  source 1..26: mol_type = other RNA
tctgcaggccagatgagggctccaga

SEQ ID NO: 52 - RNA, 26 nt, synthetic construct
  source 1..26: mol_type = other RNA
gagccagagaggatcctgggagggag

SEQ ID NO: 53 - RNA, 26 nt, synthetic construct
  source 1..26: mol_type = other RNA
gaccactgtgtgggggtaaaggacct

SEQ ID NO: 54 - RNA, 26 nt, synthetic construct
  source 1..26: mol_type = other RNA
cccccatttcctggagccatctctct

SEQ ID NO: 55 - RNA, 26 nt, synthetic construct
  source 1..26: mol_type = other RNA
aaccttagaggttctggcaaggagag

SEQ ID NO: 56 - RNA, 26 nt, synthetic construct
  source 1..26: mol_type = other RNA
agcaaaccttagaggttctggcaagg

SEQ ID NO: 57 - RNA, 26 nt, synthetic construct
  source 1..26: mol_type = other RNA
atggagccagagaggatcctgggagg

SEQ ID NO: 58 - RNA, 26 nt, synthetic construct
  source 1..26: mol_type = other RNA
ggagggaagggggggatgcgtgacct

SEQ ID NO: 59 - RNA, 26 nt, synthetic construct
  source 1..26: mol_type = other RNA
tgacctgcccggttctcagtggccac

SEQ ID NO: 60 - RNA, 26 nt, synthetic construct
  source 1..26: mol_type = other RNA
gagccagagaggatcctgggagggag

SEQ ID NO: 61 - RNA, 24 nt, synthetic construct
  source 1..24: mol_type = other RNA
gagccagagaggatcctgggaggg

SEQ ID NO: 62 - RNA, 22 nt, synthetic construct
  source 1..22: mol_type = other RNA
gagccagagaggatcctgggag

SEQ ID NO: 63 - RNA, 21 nt, synthetic construct
  source 1..21: mol_type = other RNA
gagccagagaggatcctggga

SEQ ID NO: 64 - RNA, 20 nt, synthetic construct
  source 1..20: mol_type = other RNA
gagccagagaggatcctggg

SEQ ID NO: 65 - RNA, 19 nt, synthetic construct
  source 1..19: mol_type = other RNA
gagccagagaggatcctgg

SEQ ID NO: 66 - RNA, 18 nt, synthetic construct
  source 1..18: mol_type = other RNA
gagccagagaggatcctg

SEQ ID NO: 67 - RNA, 16 nt, synthetic construct
  source 1..16: mol_type = other RNA
gagccagagaggatcc

(19) United States
(12) Patent Application Publication
(10) Pub. No.: US 2025/0368974 A1
(43) Pub. Date: Dec. 4, 2025

(54) Novel CRISPR-Cas delta enzyme and system

(71) Applicant: CHINA AGRICULTURAL UNIVERSITY, Beijing (CN)

(72) Inventors: Jinsheng LAI, Beijing (CN); Zhijia YANG, Beijing (CN); Meixia YU, Beijing (CN); Jian CHEN, Beijing (CN); Beibei XIN, Beijing (CN); Yunpeng TENG, Beijing (CN); Yueheng ZHOU, Beijing (CN); Haiming ZHAO, Beijing (CN); Weibin SONG, Beijing (CN)

(21) Appl. No.: 18/990,889

(22) Filed: Dec. 20, 2024

Related U.S. Application Data
(63) Continuation of application No. PCT/CN2024/116796, filed on Sep. 4, 2024.

(30) Foreign Application Priority Data
Sep. 4, 2023 (CN) 202311130084.6

(51) Int. Cl. C12N 9/22; C12N 15/11; C12Q 1/34; C12Q 1/6823; C12Q 1/6886; C12Q 1/6888

(52) U.S. Cl. CPC C12N 9/222; C12N 15/111; C12Q 1/34; C12Q 1/6823; C12Q 1/6886; C12Q 1/6888; C07K 2319/09; C07K 2319/40; C12N 2310/20; G01N 2333/922

(57) ABSTRACT

The present invention relates to the field of nucleic acid editing, in particular to the field of clustered regularly interspaced short palindromic repeat (CRISPR) technology. Specifically, the present invention relates to Cas effector proteins, fusion proteins comprising such proteins, and nucleic acid molecules encoding them. The present invention also relates to complexes and compositions for nucleic acid editing (e.g., gene or genome editing), which comprise the proteins or fusion proteins of the present invention, or nucleic acid molecules encoding them. The present invention also relates to a method for nucleic acid editing (e.g., gene or genome editing), which uses the proteins or fusion proteins comprising the present invention.

### CROSS-REFERENCE-RELATED APPLICATIONS

The present application is a 371 National Phase of International Application No. PCT/CN2024/116796, filed Sep. 4, 2024, which claims the priority of Chinese patent application No. 202311130084.6 filed on Sep. 4, 2023, and the entire contents of the patent applications are incorporated herein by reference in their entirety.

### SEQUENCE LISTING

The instant application contains a Sequence Listing which has been submitted electronically in XML file format and is hereby incorporated by reference in its entirety. Said XML file, created on Dec. 17, 2024, is named IEC240568PUS_Sequence Listing.xml and is 82,362 bytes in size.

### TECHNICAL FIELD

The present invention relates to nucleic acid editing, particularly the field of regularly clustered interspaced short palindromic repeats (CRISPR) technology. Specifically, the present invention relates to Cas effector proteins, fusion proteins comprising such proteins, and nucleic acid molecules encoding them. The present invention also relates to complexes and compositions for nucleic acid editing (e.g., gene or genome editing), which comprise proteins or fusion proteins of the present invention or nucleic acid molecules encoding them. The present invention also relates to methods for nucleic acid editing (e.g., gene or genome editing), which use proteins or fusion proteins comprising those of the present invention.

### BACKGROUND

CRISPR/Cas technology is a widely used gene editing technique that utilizes biological non-homologous end joining or homologous recombination to perform site-directed gene editing by specifically binding to target sequences on the genome through RNA guidance and cutting the DNA to produce double-strand breaks.

CRISPR/Cas9 system is the most commonly used type II CRISPR system, which recognizes 3′-NGG PAM motifs and performs blunt-end cutting on the target sequences. CRISPR/Cas Type V system is a newly discovered CRISPR system in the last two years, which has a 5′-TTN motif and performs sticky-end cutting on the target sequence, and examples include Cpf1, C2c1, CasX, and CasY. However, different CRISPR/Cas currently have distinct advantages and disadvantages. For example, Cas9, C2c1, and CasX all require two RNAs for guide RNA, while Cpf1 requires only one guide RNA and can be used for multiple gene editing. CasX has a size of 980 amino acids, while the common Cas9, C2c1, CasY, and Cpf1 are usually around 1300 amino acids in size. In addition, the PAM sequences of Cas9, Cpf1, CasX, and CasY are relatively complex and diverse. At the same time, C2c1 recognizes a rigorous 5′-TTN, so its target site is easier to predict than other systems, thereby reducing potential off-target effects.

In summary, given that currently available CRISPR/Cas systems are limited by some shortcomings, developing a new CRISPR/Cas system that is more robust and has good performance in many aspects is of great significance to the development of biotechnology.

### CONTENTS OF THE INVENTION

After extensive experiments and repeated explorations, the inventor of the present application unexpectedly discovered a new type of RNA-guided endonuclease. The inventors then developed a new CRISPR/Cas system and a gene editing method based on this system.

### Cas Effector Protein

Therefore, in the first aspect, the present invention provides a protein having an amino acid sequence as set forth in any one of SEQ ID NOs: 1, 2, and 3 or ortholog, homolog, variant, functional fragment thereof; wherein the ortholog, homolog, variant or functional fragment substantially retain the biological function of the sequence from which it is derived.

In the present invention, the biological function of the above sequence includes, but is not limited to, the activity of binding to a guide RNA, the activity of endonuclease, and the activity of binding to and cutting a specific site of a target sequence under the guidance of a guide RNA.

In certain embodiments, the ortholog, homolog, variant has a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence from which it is derived.

In certain embodiments, the ortholog, homolog, variant has a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in any one of SEQ ID NO: 1, 2, and 3, and substantially retains the biological function of the sequence from which it is derived (e.g., the activity of binding to a guide RNA, the activity of endonuclease, the activity of binding to and cutting a specific site of a target sequence under the guidance of a guide RNA).

In certain embodiments, the protein is an effector protein in a CRISPR/Cas system.

In certain embodiments, the protein of the present invention comprises or consists of a sequence selected from the following:
(i) a sequence as set forth in any one of SEQ ID NOs: 1, 2, and 3;
(ii) a sequence having a substitution, deletion, or addition of one or more amino acids (e.g., a substitution, deletion, or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 amino acids) as compared to the sequence as set forth in any one of SEQ ID NOs: 1, 2, and 3; or
(iii) a sequence having a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in any one of SEQ ID NOs: NO: 1, 2, and 3.

### Truncated Protein

In a second aspect, the present invention provides a truncated protein, in which the truncated protein has a truncation of one or more amino acids (e.g., 1 to 10, 11 to 20, 21 to 30, 31 to 40, 41 to 50, 51 to 60, or more amino acids) at the N-terminal and/or C-terminal as compared to the protein described in the first aspect.

In certain embodiments, the truncated protein has a truncation of 31 amino acids at the N-terminal as compared to the protein described in the first aspect.

In certain embodiments, the truncated protein has a truncation of 31 amino acids at the N-terminal as compared to the sequence as set forth in any one of SEQ ID NO: 1 and 2.

In certain embodiments, the term “a truncation of 31 amino acids at the N-terminal” refers to truncating 31 amino acids consecutively from the starting amino acid at the N-terminal.

In certain embodiments, the truncated protein comprises or consists of a sequence selected from the following:
(i) a sequence as set forth in SEQ ID NO: 3;
(ii) a sequence having a substitution, deletion, or addition of one or more amino acids (e.g., a substitution, deletion, or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 amino acids) as compared to the sequence as set forth in SEQ ID NO: 3; or
(iii) a sequence having a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in SEQ ID NO: 3.

In certain embodiments, the truncated protein has the amino acid sequence as set forth in SEQ ID NO: 3.

### Derivatized Protein

The protein or truncated protein of the present invention can be derivatized, for example, connected to another molecule (e.g., another polypeptide or protein). Generally, the derivatization (e.g., labeling) of a protein does not adversely affect the desired activity of the protein (e.g., activity of binding to a guide RNA, activity of endonuclease, activity of binding to and cutting a specific site of a target sequence under the guidance of a guide RNA). Therefore, the protein of the present invention is also intended to include such derivatized forms. For example, the protein of the present invention can be functionally connected (by chemical coupling, gene fusion, non-covalent connection or other means) to one or more other molecular groups, such as another protein or polypeptide, a detection agent, a pharmaceutical agent, etc.

In particular, the protein of the present invention can be linked to another functional unit. For example, it can be linked to a nuclear localization signal (NLS) sequence to improve the ability of the protein of the present invention to enter the cell nucleus. For example, it can be linked to a targeting moiety to endow the protein of the present invention with targeting ability. For example, it can be linked to a detectable label to facilitate the detection of the protein of the present invention. For example, it can be linked to an epitope tag to facilitate the expression, detection, tracing and/or purification of the protein of the present invention.

### Conjugate

Therefore, in a third aspect, the present invention provides a conjugate, which comprises the protein or truncated protein as described above and a modified moiety.

In certain embodiments, the modified moiety is selected from the group consisting of an additional protein or polypeptide, a detectable label, and any combination thereof.

In certain embodiments, the additional protein or polypeptide is selected from the group consisting of an epitope tag, a reporter gene sequence, a nuclear localization signal (NLS) sequence, a targeting moiety, a transcriptional activation domain (e.g., VP64), a transcriptional repression domain (e.g., a KRAB domain or a SID domain), a nuclease domain (e.g., Fok1), a domain having an activity selected from the following: nucleotide deaminase, methylase activity, demethylase, transcriptional activation activity, transcriptional repression activity, transcriptional release factor activity, histone modification activity, nuclease activity, single-stranded RNA cleavage activity, double-stranded RNA cleavage activity, single-stranded DNA cleavage activity, double-stranded DNA cleavage activity and nucleic acid binding activity, and any combination thereof.

In certain embodiments, the conjugate of the present invention comprises one or more NLS sequences, such as a NLS of large T antigen of SV40 virus. In certain exemplary embodiments, the NLS sequence is set forth in SEQ ID NO: 27. In certain embodiments, the NLS sequence is located at, near or close to the end (e.g., N-terminal or C-terminal) of the protein or truncated protein of the present invention. In certain exemplary embodiments, the NLS sequence is located at, near, or close to the C-terminal of the protein or truncated protein of the present invention.

In certain embodiments, the conjugate of the present invention comprises an epitope tag. Such epitope tags are well known to those skilled in the art, and examples thereof include but are not limited to, His, V5, FLAG, HA, Myc, VSV to G, Trx, etc., and those skilled in the art know how to select a suitable epitope tag according to the desired purpose (e.g., purification, detection or tracing).

In certain embodiments, the conjugate of the present invention comprises a reporter gene sequence. Such reporter genes are well known to those skilled in the art, and examples thereof include but are not limited to, GST, HRP, CAT, GFP, HcRed, DsRed, CFP, YFP, BFP, etc.

In certain embodiments, the conjugate of the present invention comprises a domain capable of binding to a DNA molecule or an intracellular molecule, such as a maltose binding protein (MBP), a DNA binding domain (DBD) of Lex A, a DBD of GAL4, etc.

In certain embodiments, the conjugate of the present invention comprises a detectable label, such as a fluorescent dye, such as FITC or DAPI.

In certain embodiments, the protein or truncated protein of the present invention is coupled, conjugated, or fused to the modification portion optionally via a linker.

In certain embodiments, the modification portion is directly connected to the N-terminal or C-terminal of the protein or truncated protein of the present invention.

In certain embodiments, the modification portion is connected to the N-terminal or C-terminal of the protein or truncated protein of the present invention via a linker. Such linkers are well known in the art, and examples thereof include but are not limited to, linkers comprising one or more (e.g., 1, 2, 3, 4 or 5) amino acids (e.g., Glu or Ser) or amino acid derivatives (e.g., Ahx, β-Ala, GABA or Ava), or PEG, etc.

### Fusion Protein

In a fourth aspect, the present invention provides a fusion protein, which comprises the protein described in the first aspect or the truncated protein described in the second aspect and an additional protein or polypeptide.

In certain embodiments, the additional protein or polypeptide is selected from the group consisting of an epitope tag, a reporter gene sequence, a nuclear localization signal (NLS) sequence, a targeting moiety, a transcriptional activation domain (e.g., VP64), a transcriptional repression domain (e.g., a KRAB domain or a SID domain), a nuclease domain (e.g., Fok1), a domain having an activity selected from the following: nucleotide deaminase, methylase activity, demethylase, transcriptional activation activity, transcriptional repression activity, transcriptional release factor activity, histone modification activity, nuclease activity, single-stranded RNA cleavage activity, double-stranded RNA cleavage activity, single-stranded DNA cleavage activity, double-stranded DNA cleavage activity and nucleic acid binding activity, and any combination thereof.

In certain embodiments, the fusion protein of the present invention comprises one or more NLS sequences, such as a NLS of large T antigen of SV40 virus. In certain embodiments, the NLS sequence is located at, near or close to the end (e.g., N-terminal or C-terminal) of the protein or truncated protein of the present invention. In certain exemplary embodiments, the NLS sequence is located at, near or close to the C-terminal of the protein or truncated protein of the present invention.

In certain embodiments, the fusion protein of the present invention comprises an epitope tag.

In certain embodiments, the fusion protein of the present invention comprises a reporter gene sequence.

In certain embodiments, the fusion protein of the present invention comprises a domain capable of binding to a DNA molecule or an intracellular molecule.

In certain embodiments, the protein or truncated protein of the present invention is fused to the additional protein or polypeptide optionally via a linker.

In certain embodiments, the additional protein or polypeptide is directly connected to the N-terminal or C-terminal of the protein or truncated protein of the present invention.

In certain embodiments, the additional protein or polypeptide is connected to the N-terminal or C-terminal of the protein or truncated protein of the present invention via a linker.

In certain exemplary embodiments, the fusion protein of the present invention has an amino acid sequence as set forth in any one of SEQ ID NOs: 28 to 30.

The protein of the present invention, the conjugate of the present invention or the fusion protein of the present invention is not limited by the production method thereof, for example, it can be produced by a genetic engineering method (recombinant technology) or by a chemical synthesis method.

### Direct Repeat Sequence

In the fifth aspect, the present invention provides an isolated nucleic acid molecule, which comprises or consists of a sequence selected from the following sequences:
(i) a sequence as set forth in any one of SEQ ID NOs: 7, 8, 9, 10, 11, 12, 13, 14, 15 or 16;
(ii) a sequence having a substitution, deletion or addition of one or more bases (e.g., a substitution, deletion or addition of 1, 2, 3, 4, 5, 6, 7, 8, 9 or 10 bases) as compared to the sequence as set forth in any one of SEQ ID NOs: 7, 8, 9, 10, 11, 12, 13, 14, 15 or 16;
(iii) a sequence having a sequence identity of at least 20%, at least 30%, at least 40%, at least 50%, at least 60%, at least 70%, at least 80%, at least 90%, at least 95% as compared to the sequence as set forth in any one of SEQ ID NOs: 7, 8, 9, 10, 11, 12, 13, 14, 15 or 16;
(iv) a sequence capable of hybridizing with the sequence as described in any one of (i) to (iii) under a stringent condition; or
(v) a complementary sequence of the sequence as described in any one of (i) to (iii);
and, the sequence as described in any one of (ii) to (v) substantially retains the biological function of the sequence from which it is derived, and the biological function of the sequence refers to the activity as a direct repeat sequence in the CRISPR-Cas system.

In certain embodiments, the isolated nucleic acid molecule is a direct repeat sequence in the CRISPR-Cas system.

In certain embodiments, the nucleic acid molecule comprises a sequence selected from the following, or consists of a sequence selected from the following:
(a) a nucleotide sequence as set forth in any one of SEQ ID NOs: 7, 8, 9, 10, 11, 12, 13, 14, 15 or 16;
(b) a sequence capable of hybridizing with the sequence as described in (a) under a stringent condition; or
(c) a complementary sequence of the sequence as described in (a).

In certain embodiments, the isolated nucleic acid molecule is an RNA. In certain embodiments, the isolated nucleic acid molecule is a direct repeat sequence in the CRISPR/Cas system.

### CRISPR/Cas Complex

In a sixth aspect, the present invention provides a complex, which comprises:
(i) a protein component, which is selected from the group consisting of the protein, truncated protein, conjugate or fusion protein of the present invention, and any combination thereof; and
(ii) a nucleic acid component, which comprises, from the 5′ to 3′ direction, the isolated nucleic acid molecule as described above and a guide sequence capable of hybridizing with a target sequence,
wherein the protein component and the nucleic acid component bind to each other to form a complex.

In some embodiments, the guide sequence is ligated to the 3′ end of the nucleic acid molecule.

In some embodiments, the guide sequence comprises a complementary sequence to the target sequence.

In some embodiments, the nucleic acid component is a guide RNA in a CRISPR-Cas system.

In some embodiments, the nucleic acid molecule is an RNA.

In some embodiments, the complex does not comprise a trans-activating crRNA (tracrRNA).

In some embodiments, the guide sequence has a length of at least 5, at least 10, at least 15, at least 20, at least 25, at least 30 nucleotides. In some embodiments, the guide sequence has a length of 10 to 30, or 15 to 25, or 15 to 22, or 19 to 25, or 19 to 22 nucleotides.

In some embodiments, the isolated nucleic acid molecule has a length of 55 to 70 nucleotides, such as 55 to 65 nucleotides, such as 60 to 65 nucleotides, such as 62 to 65 nucleotides, such as 63 to 64 nucleotides. In some embodiments, the isolated nucleic acid molecule has a length of 15 to 30 nucleotides, such as 15 to 25 nucleotides, such as 20 to 25 nucleotides, such as 22 to 24 nucleotides, such as 23 nucleotides.

### Encoding Nucleic Acid, Vector and Host Cell

In the seventh aspect, the present invention provides an isolated nucleic acid molecule, which comprises:
(i) a nucleotide sequence encoding the protein, truncated protein or fusion protein of the present invention;
(ii) a nucleotide sequence encoding the isolated nucleic acid molecule as described in the fifth aspect; or
(iii) a nucleotide sequence comprising (i) and (ii).

In some embodiments, the nucleotide sequence as described in any one of (i) to (iii) is optimized with a codon for expression in prokaryotic cells. In some embodiments, the nucleotide sequence as described in any one of (i) to (iii) is optimized with a codon for expression in eukaryotic cells.

In the eighth aspect, the present invention also provides a vector, which comprises the isolated nucleic acid molecule as described in the seventh aspect. The vector of the present invention can be a cloning vector or an expression vector. In certain embodiments, the vector of the present invention is, for example, a plasmid, a cosmid, a phage, a Kos plasmid, and the like. In certain embodiments, the vector is capable of expressing the protein, truncated protein, fusion protein, isolated nucleic acid molecule as described in the fifth aspect, or the complex as described in the sixth aspect of the present invention in a subject (e.g., a mammal, such as a human).

In the ninth aspect, the present invention also provides a host cell comprising the isolated nucleic acid molecule or vector as described above. Such host cells include, but are not limited to, prokaryotic cells such as Escherichia coli cells, and eukaryotic cells such as yeast cells, insect cells, plant cells (e.g., cassava, corn, sorghum, soybean, wheat, oat or rice cells) and animal cells (e.g., mammalian cells, such as mouse cells, human cells, etc.). The cell of the present invention can also be a cell line, such as a 293T cell.

### Composition and Vector Composition

In the tenth aspect, the present invention also provides a composition, which comprises:
(i) a first component, which is selected from the group consisting of the protein, truncated protein, conjugate, fusion protein of the present invention, or a nucleotide sequence encoding the protein, truncated protein or fusion protein, and any combination thereof; and
(ii) a second component, which is a nucleotide sequence comprising a guide RNA, or a nucleotide sequence encoding the nucleotide sequence comprising the guide RNA;
wherein, the guide RNA comprises a direct repeat sequence and a guide sequence from the 5′ to 3′ direction, and the guide sequence is capable of hybridizing with a target sequence;
the guide RNA is capable of forming a complex with the protein, truncated protein, conjugate or fusion protein as described in (i).

In certain embodiments, the direct repeat sequence is the isolated nucleic acid molecule as defined in the fifth aspect.

In certain embodiments, the guide sequence is ligated to the 3′ end of the direct repeat sequence. In certain embodiments, the guide sequence comprises a complementary sequence of the target sequence.

In certain embodiments, the composition does not comprise a trans-activating crRNA (tracrRNA).

In certain embodiments, the composition is non-naturally occurring or modified. In certain embodiments, at least one component of the composition is non-naturally occurring or modified. In certain embodiments, the first component is non-naturally occurring or modified; and/or, the second component is non-naturally occurring or modified.

In certain embodiments, when the target sequence is DNA, the target sequence is located at the 3′ end of the protospacer adjacent motif (PAM), and the PAM has a sequence shown as 5′-RYR, wherein R is selected from A or G, and Y is selected from T or C. In certain embodiments, the sequence of the PAM is selected from the group consisting of ATG, ACG, GTG, ATA, ACA, GCA, GTA and/or GCG.

In certain embodiments, when the target sequence is RNA, the target sequence does not have a PAM domain restriction.

In certain embodiments, the target sequence is a DNA or RNA sequence from a prokaryotic cell or a eukaryotic cell. In certain embodiments, the target sequence is a non-naturally occurring DNA or RNA sequence.

In certain embodiments, the target sequence is present in a cell. In certain embodiments, the target sequence is present in the nucleus or the cytoplasm (e.g., an organelle). In certain embodiments, the cell is a eukaryotic cell. In certain embodiments, the cell is a prokaryotic cell.

In certain embodiments, the protein or truncated protein is linked to one or more NLS sequences. In certain embodiments, the conjugate or fusion protein comprises one or more NLS sequences. In certain embodiments, the NLS sequence is linked to the N-terminal or C-terminal of the protein. In certain embodiments, the NLS sequence is fused to the N-terminal or C-terminal of the protein.

In the eleventh aspect, the present invention also provides a composition, which comprises one or more vectors, wherein the one or more vectors comprise:
(i) a first nucleic acid, which comprises a nucleotide sequence encoding the protein, truncated protein or fusion protein of the present invention; optionally, the first nucleic acid is operably linked to a first regulatory element; and
(ii) a second nucleic acid, which comprises a nucleotide sequence encoding a guide RNA; optionally, the second nucleic acid is operably linked to a second regulatory element;
wherein:
the first nucleic acid and the second nucleic acid are present on the same vector or different vectors;
the guide RNA comprises a direct repeat sequence and a guide sequence from the 5′ to 3′ direction, and the guide sequence is capable of hybridizing with a target sequence;
the guide RNA is capable of forming a complex with the protein, truncated protein or fusion protein as described in (i).

In certain embodiments, the direct repeat sequence is an isolated nucleic acid molecule as defined in the fifth aspect.

In certain embodiments, the guide sequence is linked to the 3′ end of the direct repeat sequence. In certain embodiments, the guide sequence comprises a complementary sequence of the target sequence.

In certain embodiments, the composition does not comprise a trans-activating crRNA (tracrRNA).

In certain embodiments, the composition is non-naturally occurring or modified. In some embodiments, at least one component of the composition is non-naturally occurring or modified.

In some embodiments, the first regulatory element is a promoter, such as an inducible promoter.

In some embodiments, the second regulatory element is a promoter, such as an inducible promoter.

In some embodiments, when the target sequence is DNA, the target sequence is located at the 3′ end of the protospacer adjacent motif (PAM), and the PAM has a sequence shown as 5′-RYR, wherein R is A or G, and Y is T or C.

In some embodiments, the sequence of the PAM is selected from the group consisting of ATG, ACG, GTG, ATA, ACA, GCA, GTA and/or GCG.

In some embodiments, when the target sequence is RNA, and the target sequence does not have a PAM domain restriction.

In some embodiments, the target sequence is a DNA or RNA sequence from a prokaryotic cell or a eukaryotic cell. In some embodiments, the target sequence is a non-naturally occurring DNA or RNA sequence.

In some embodiments, the target sequence is present in a cell. In certain embodiments, the target sequence is present in the nucleus or the cytoplasm (e.g., an organelle). In certain embodiments, the cell is a eukaryotic cell. In certain embodiments, the cell is a prokaryotic cell.

In certain embodiments, the protein is linked to one or more NLS sequences. In certain embodiments, the conjugate or fusion protein comprises one or more NLS sequences. In certain embodiments, the NLS sequence is linked to the N-terminal or C-terminal of the protein. In certain embodiments, the NLS sequence is fused to the N-terminal or C-terminal of the protein.

In certain embodiments, one type of vector is a plasmid, which refers to a circular double-stranded DNA loop into which an additional DNA fragment can be inserted, for example, by standard molecular cloning techniques. Another type of vector is a viral vector, in which a virally derived DNA or RNA sequence is present in a vector for packaging a virus (e.g., a retrovirus, a replication-defective retrovirus, an adenovirus, a replication-defective adenovirus, and an adeno-associated virus). The viral vector further comprises a polynucleotide carried by a virus for transfection into a host cell. Certain vectors (e.g., bacterial vectors with bacterial replication origin, and episomal mammalian vectors) are capable of autonomous replication in host cells into which they are introduced. Other vectors (e.g., non-episomal mammalian vectors) are integrated into the genome of a host cell upon introduction into the host cell and are thereby replicated along with the host genome. Moreover, certain vectors are capable of directing the expression of genes to which they are operably linked. Such vectors are referred to herein as “expression vectors”. Common expression vectors used in recombinant DNA technology are generally in the form of plasmids.

The recombinant expression vector may comprise the nucleic acid molecule of the present invention in a form suitable for nucleic acid expression in a host cell, meaning that such recombinant expression vector comprises one or more regulatory elements selected based on the host cell to be used for expression, and the regulatory elements are operably linked to the nucleic acid sequence to be expressed.

### Delivery and Delivery Composition

The protein of the present invention, the truncated protein of the present invention, the conjugate of the present invention, the fusion protein of the present invention, the isolated nucleic acid molecule as described in the fifth aspect, the complex of the present invention, the isolated nucleic acid molecule as described in the seventh aspect, the vector as described in the eighth aspect, and the composition as described in the tenth and eleventh aspects of the present invention may be delivered by any method known in the art. Such methods include but are not limited to, electroporation, lipofection, nucleofection, microinjection, sonoporation, gene gun, calcium phosphate-mediated transfection, cationic transfection, liposome transfection, dendritic transfection, heat shock transfection, nucleofection, magnetofection, lipofection, puncture transfection, optical transfection, agent-enhanced nucleic acid uptake, and delivery via liposome, immunoliposome, viral particle, artificial virion, etc.

Therefore, in the twelfth aspect, the present invention provides a delivery composition, which comprises a delivery vector, and one or more selected from the following: the protein, truncated protein, conjugate, fusion protein, isolated nucleic acid molecule as described in the fifth aspect, the complex of the present invention, the isolated nucleic acid molecule as described in the seventh aspect, the vector as described in the eighth aspect, and the composition as described in the tenth aspect and the eleventh aspect.

In some embodiments, the delivery vector is a particle.

In some embodiments, the delivery vector is selected from the group consisting of lipid particle, sugar particle, metal particle, protein particle, liposome, exosome, microvesicle, gene gun, or viral vector (e.g., replication-defective retrovirus, lentivirus, adenovirus, or adeno-associated virus).

### Kit

In another aspect, the present invention provides a kit, which comprises one or more of the components described above. In certain embodiments, the kit comprises one or more components selected from the following: the protein of the present invention, the truncated protein of the present invention, the conjugate of the present invention, the fusion protein of the present invention, the isolated nucleic acid molecule as described in the fifth aspect, the complex of the present invention, the isolated nucleic acid molecule as described in the seventh aspect, the vector as described in the eighth aspect, and the composition as described in the tenth and eleventh aspects of the present invention.

In certain embodiments, the kit of the present invention comprises the composition as described in the tenth aspect. In certain embodiments, the kit further comprises instructions for using the composition.

In certain embodiments, the kit of the present invention comprises the composition as described in the eleventh aspect. In certain embodiments, the kit further comprises instructions for using the composition.

In certain embodiments, the components contained in the kit of the present invention can be provided in any suitable container.

In certain embodiments, the kit further comprises one or more buffers. The buffers can be any buffer, including but not limited to sodium carbonate buffer, sodium bicarbonate buffer, borate buffer, Tris buffer, MOPS buffer, HEPES buffer, and combinations thereof. In certain embodiments, the buffer is alkaline. In certain embodiments, the buffer has a pH of about 7 to about 10.

In certain embodiments, the kit further comprises one or more oligonucleotides, and one or more oligonucleotides correspond to a guide sequence for insertion into a vector to operably link the guide sequence and the regulatory element. In certain embodiments, the kit comprises a homologous recombination template polynucleotide.

### Method and Use

In another aspect, the present invention provides a method to modify a target gene, which comprises: contacting the complex of the present invention, the composition as described in the tenth aspect and the eleventh aspect with the target gene, or delivering it to a cell containing the target gene; wherein the target sequence is present in the target gene.

In some embodiments, the method is used for modifying the target gene in vitro or ex vivo. In some embodiments, the method is not a method for treating a human or animal via therapy. In some embodiments, the method does not comprise a step of modifying a human germline genetic characteristic.

In some embodiments, the target gene is present in a cell. In some embodiments, the cell is a prokaryotic cell. In some embodiments, the cell is a eukaryotic cell. In some embodiments, the cell is a mammalian cell. In some embodiments, the cell is a human cell. In some embodiments, the cell is selected from the group consisting of non-human cell primate cell, bovine cell, porcine cell or rodent cell. In some embodiments, the cell is a non-mammalian eukaryotic cell, such as poultry or fish cell. In some embodiments, the cell is a plant cell, such as a cell of a cultivated plant (e.g., cassava, corn, sorghum, wheat or rice), algae, tree or vegetable.

In some embodiments, the target gene is present in a nucleic acid molecule (e.g., a plasmid) in vitro. In some embodiments, the target gene is present in a plasmid.

In some embodiments, the method results in a breakage in the target sequence (e.g., a double-strand breakage in DNA or a single-strand breakage in RNA). In some embodiments, the breakage results in a reduced transcription of the target gene.

In some embodiments, the method further comprises: contacting an editing template (e.g., an exogenous nucleic acid) with the target gene, or delivering it to a cell comprising the target gene. In such embodiments, the method repairs the broken target gene by homologous recombination with an editing template (e.g., an exogenous nucleic acid), wherein the repair results in a mutation comprising an insertion, deletion, or substitution of one or more nucleotides of the target gene. In some embodiments, the mutation results in one or more amino acid changes in a protein expressed from a gene comprising the target sequence.

Thus, in some embodiments, the modification further comprises inserting an editing template (e.g., an exogenous nucleic acid) into the breakage.

In certain embodiments, the protein, truncated protein, conjugate, fusion protein, isolated nucleic acid molecule, complex, vector or composition is contained in a delivery vehicle.

In certain embodiments, the delivery vehicle is selected from the group consisting of lipid particles, sugar particles, metal particles, protein particles, liposomes, exosomes, viral vectors (e.g., replication-defective retroviruses, lentiviruses, adenoviruses or adeno-associated viruses).

In certain embodiments, the method is used to modify a cell, cell line or organism by changing one or more target sequences in a target gene or a nucleic acid molecule encoding a target gene product.

In another aspect, the present invention provides a method for changing the expression of a gene product, which comprises: contacting the complex of the present invention, the composition as described in the tenth aspect and the eleventh aspect with a nucleic acid molecule encoding the gene product, or delivering it to a cell comprising the nucleic acid molecule; wherein the target sequence is present in the nucleic acid molecule.

In certain embodiments, the method is used to change the expression of a gene product in vitro or ex vivo. In certain embodiments, the method is not a method for treating a human or animal by therapy. In certain embodiments, the method does not comprise a step of modifying a human germline genetic characteristic.

In certain embodiments, the nucleic acid molecule is present in a cell. In some embodiments, the cell is a prokaryotic cell. In some embodiments, the cell is a eukaryotic cell. In some embodiments, the cell is a mammalian cell. In some embodiments, the cell is a human cell. In some embodiments, the cell is selected from the group consisting of non-human primate cell, bovine cell, porcine cell or rodent cell. In some embodiments, the cell is a non-mammalian eukaryotic cell, such as poultry or fish cell. In some embodiments, the cell is a plant cell, such as a cell of a cultivated plant (e.g., cassava, corn, sorghum, wheat or rice), algae, tree or vegetable.

In some embodiments, the nucleic acid molecule is present in a nucleic acid molecule (e.g., a plasmid) in vitro. In some embodiments, the nucleic acid molecule is present in a plasmid.

In some embodiments, the expression of the gene product is altered (e.g., enhanced or reduced). In some embodiments, the expression of the gene product is enhanced. In some embodiments, the expression of the gene product is reduced.

In some embodiments, the gene product is a protein.

In certain embodiments, the protein, truncated protein, conjugate, fusion protein, isolated nucleic acid molecule, complex, vector or composition is contained in a delivery vehicle.

In certain embodiments, the delivery vehicle is selected from the group consisting of lipid particles, sugar particles, metal particles, protein particles, liposomes, exosomes, viral vectors (e.g., replication-defective retroviruses, lentiviruses, adenoviruses or adeno-associated viruses).

In certain embodiments, the method is used for modifying a cell, a cell line or an organism by changing one or more target sequences in a target gene or a nucleic acid molecule encoding a target gene product.

In another aspect, the present invention relates to a use of the protein as described in the first aspect, the truncated protein as described in the second aspect, the conjugate as described in the third aspect, the fusion protein as described in the fourth aspect, the isolated nucleic acid molecule as described in the fifth aspect, the complex as described in the sixth aspect, the isolated nucleic acid molecule as described in the seventh aspect, the vector as described in the eighth aspect, the composition as described in the tenth aspect, the composition as described in the eleventh aspect, the kit of the present invention, in the manufacture of a preparation, in which the preparation is used for nucleic acid editing (e.g., in vitro or ex vivo nucleic acid editing).

In certain embodiments, the nucleic acid to be edited is present in a cell. In some embodiments, the cell is a prokaryotic cell or a eukaryotic cell. In some embodiments, the nucleic acid to be edited is present in a nucleic acid molecule (e.g., a plasmid) in vitro.

In some embodiments, the nucleic acid editing comprises gene or genome editing, such as modifying a gene, knocking out a gene, changing the expression of a gene product, repairing a mutation, and/or inserting a polynucleotide. In some embodiments, the gene or genome editing does not comprise a step of modifying a human germline genetic characteristic. In some embodiments, the use is not a method of treating a human or animal by therapy.

In some embodiments, the use further comprises repairing the edited target sequence by homologous recombination with an exogenous template polynucleotide, wherein the repair can produce a mutation of the target sequence, which comprises an insertion, deletion, or substitution of one or more nucleotides.

In another aspect, the present invention relates to a use of the protein as described in the first aspect, the truncated protein as described in the second aspect, the conjugate as described in the third aspect, the fusion protein as described in the fourth aspect, the isolated nucleic acid molecule as described in the fifth aspect, the complex as described in the sixth aspect, the isolated nucleic acid molecule as described in the seventh aspect, the vector as described in the eighth aspect, the composition as described in the tenth aspect, the composition as described in the eleventh aspect, the kit of the present invention, in the manufacture of a preparation, in which the preparation is used for: (i) in vitro or ex vivo DNA detection; (ii) editing a target sequence in a target locus to modify an organism or a non-human organism (e.g., a prokaryotic organism).

In certain embodiments, the preparation is used for the detection of single-stranded DNA or double-stranded DNA (e.g., detection of single-stranded or double-stranded DNA in a prokaryotic cell).

In certain embodiments, the DNA detection is used to detect a tumor, a virus or a bacterium. Without being limited by theory, it is believed that due to the non-specific cleavage characteristics of Casδ protein on single-stranded DNA after target DNA recognition, when a target DNA (e.g., a tumor-specific label, virus or bacterium-specific label) is present, it is possible to achieve the detection of tumor, Ebola virus, avian influenza virus, African swine fever virus and other viruses or bacteria by adding a detectable single-stranded DNA and detecting the non-specific cleavage of the single-stranded DNA.

On the other hand, the present invention also provides a method for detecting whether a target nucleic acid is present in a sample, which comprises the following steps:
(1) contacting the sample with a labeled DNA probe and any of the following components: the complex of the present invention, the composition as described in the tenth aspect or the eleventh aspect, or the kit of the present invention;
wherein, the guide sequence contained in the complex, composition or kit is capable of hybridizing with the target nucleic acid, and the DNA probe does not hybridize with the guide sequence;
in certain embodiments, the DNA probe emits a detectable signal after being cleaved;
(2) detecting the detectable signal generated by the cleavage of DNA probes by the protein or truncated protein contained in the complex, composition or kit cleaves the DNA probe, thereby determining whether the target nucleic acid is present in the sample.

In some embodiments, one end (e.g., 5′ end) of the DNA probe is labeled with a fluorescent group, and the other end (e.g., 3′ end) is labeled with a quenching group.

In some embodiments, the sequence of the target nucleic acid is a sequence obtained from a pathogen. In some embodiments, the pathogen is selected from the group consisting of a virus, a bacterium, a fungus, a protozoa, a parasite, or any combination thereof.

In some embodiments, the sequence of the target nucleic acid is obtained from the genome of a tumor cell.

The target nucleic acid detected in the present application can be a DNA or RNA. Therefore, in some embodiments, the method further comprises a step of contacting the sample with a reagent for reverse transcription. In some embodiments, the reagent for reverse transcription is selected from the group consisting of a reverse transcriptase, an oligonucleotide primer, a dNTP, or any combination thereof.

In some embodiments, the target nucleic acid is single-stranded or double-stranded. In some embodiments, the sequence of the target nucleic acid is a DNA or RNA sequence from a prokaryotic cell or a eukaryotic cell; or, the sequence of the target nucleic acid is a non-naturally occurring DNA or RNA sequence.

In some embodiments, the detectable signal is determined by one or more methods selected from the group consisting of imaging-based detection, sensor-based detection, color detection, gold nanoparticle-based detection, fluorescence polarization, colloidal phase transition/dispersion, electrochemical detection, and semiconductor-based sensing.

In some embodiments, the method further comprises a step of amplifying the target nucleic acid in the sample.

### Cells and Cell Progeny

In some cases, the modification introduced into the cell by the method of the present invention may cause the cell and its progeny to be altered to improve the production of its biological product (e.g., antibody, starch, ethanol or other desired cell output). In some cases, the modification introduced into the cell by the method of the present invention may cause the cell and its progeny to comprise a change that causes a change in the produced biological product.

Therefore, in another aspect, the present invention also relates to a cell or progeny thereof obtained by the method as described above, wherein the cell comprises a modification that is not present in its wild type.

The present invention also relates to a cell product of the cell or progeny thereof as described above.

The present invention also relates to an in vitro, ex vivo or in vivo cell or cell line or progeny thereof, wherein the cell or cell line or progeny thereof comprises: the protein as described in the first aspect, the truncated protein as described in the second aspect, the conjugate as described in the third aspect, the fusion protein as described in the fourth aspect, the isolated nucleic acid molecule as described in the fifth aspect, the complex as described in the sixth aspect, the isolated nucleic acid molecule as described in the seventh aspect, the vector as described in the eighth aspect, the composition as described in the tenth aspect, and the composition as described in the eleventh aspect.

In certain embodiments, the cell is a prokaryotic cell.

In certain embodiments, the cell is a eukaryotic cell. In certain embodiments, the cell is a mammalian cell. In certain embodiments, the cell is a human cell. In certain embodiments, the cell is a non-human mammalian cell, such as a cell of a non-human primate, cow, sheep, pig, dog, monkey, rabbit, or rodent (e.g., rat or mouse). In certain embodiments, the cell is a non-mammalian eukaryotic cell, such as a cell of a poultry bird (e.g., chicken), fish or crustacean (e.g., clam, shrimp). In some embodiments, the cell is a plant cell, such as a cell of a monocot or dicot plant or a cell of a cultivated plant, or a cell of a food crop, such as cassava, corn, sorghum, soybean, wheat, oat or rice, such as algae, tree or production plant, fruit or vegetable (e.g., tree, such as citrus tree, nut tree; nightshade, cotton, tobacco, tomato, grape, coffee, cocoa, etc.).

In some embodiments, the cell is a stem cell or a stem cell line.

### Definition of Terms

In the present invention, unless otherwise specified, the scientific and technical terms used herein have the meanings commonly understood by those skilled in the art. In addition, the molecular genetics, nucleic acid chemistry, chemistry, molecular biology, biochemistry, cell culture, microbiology, cell biology, genomics and recombinant DNA operation steps used herein are all conventional steps widely used in the corresponding fields. At the same time, in order to better understand the present invention, the definitions and explanations of the relevant terms are provided below.

In the present invention, the expression “Casδ” refers to a Cas effector protein first discovered and identified by the inventors, which has an amino acid sequence selected from the following:
(i) a sequence as set forth in any one of SEQ ID NOs: 1, 2, and 3;
(ii) a sequence having a substitution, deletion, or addition of one or more amino acids (e.g., a substitution, deletion, or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids) as compared to the sequence as set forth in any one of SEQ ID NOs: 1, 2, and 3; or
(iii) a sequence having a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in any one of SEQ ID NOs: 1, 2, and 3.

The Casδ of the present invention is an endonuclease that binds to and cuts a specific site of a target sequence under the guidance of a guide RNA.

As used herein, the term “clustered regularly interspaced short palindromic repeats (CRISPR)-CRISPR-associated (Cas) (CRISPR-Cas) system” or “CRISPR system” is used interchangeably and has the meaning generally understood by those skilled in the art, which generally comprises a transcription product or other element related to the expression of a CRISPR-associated (“Cas”) gene, or a transcription product or other element capable of directing the activity of the Cas gene. Such transcripts or other elements may comprise sequences encoding Cas effector proteins and guide RNAs comprising CRISPR RNA (crRNA), as well as trans-activating crRNA (tracrRNA) sequences contained in the CRISPR-Cas9 system, or other sequences or transcripts from CRISPR locus. In the Casδ-CRISPR system described in the present invention, the tracrRNA sequence is not required.

As used herein, the terms “Cas effector protein” and “Cas effector enzyme” are used interchangeably and refer to any protein greater than 800 amino acids in length presented in the CRISPR-Cas system. In some cases, such protein refers to a protein identified from the Cas locus.

As used herein, the terms “guide RNA” and “mature crRNA” are used interchangeably and have the meanings commonly understood by those skilled in the art. In general, the guide RNA may comprise a direct repeat sequence and a guide sequence, or may consist essentially of or consist of a direct repeat sequence and a guide sequence (also referred to as a spacer in the context of an endogenous CRISPR system). In some cases, the guide sequence is any polynucleotide sequence that has sufficient complementarity with a target sequence to hybridize with the target sequence and guide the specific binding of the CRISPR/Cas complex to the target sequence. In certain embodiments, when optimally aligned, the degree of complementarity between the guide sequence and corresponding target sequence thereof is at least 50%, at least 60%, at least 70%, at least 80%, at least 90%, at least 95%, or at least 99%. Determining the optimal alignment is within the capabilities of a person of ordinary skill in the art. For example, there are publicly available and commercially available alignment algorithms and programs, such as, but not limited to, ClustalW, Smith-Waterman in matlab, Bowtie, Geneious, Biopython, and SeqMan.

In some cases, the guide sequence has a length of at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, or at least 50 nucleotides. In some cases, the guide sequence has a length of no more than 50, 45, 40, 35, 30, 25, 24, 23, 22, 21, 20, 15, 10 or less nucleotides. In certain embodiments, the guide sequence has a length of 10 to 30, or 15 to 25, or 15 to 22, or 19 to 25, or 19 to 22 nucleotides.

In some cases, the direct repeat sequence has a length of at least 10, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 56, at least 57, at least 58, at least 59, at least 60, at least 61, at least 62, at least 63, at least 64, at least 65, or at least 70 nucleotides. In some cases, the direct repeat sequence has a length of no more than 70, 65, 64, 63, 62, 61, 60, 59, 58, 57, 56, 55, 50, 45, 40, 35, 30, 29, 28, 27, 26, 25, 24, 23, 22, 21, 20, 15, 10 or less nucleotides. In certain embodiments, the direct repeat sequence has a length of 55 to 70 nucleotides, such as 55 to 65 nucleotides, such as 60 to 65 nucleotides, such as 62 to 65 nucleotides, such as 63 to 64 nucleotides. In certain embodiments, the direct repeat sequence has a length of 15 to 30 nucleotides, such as 15 to 25 nucleotides, such as 20 to 25 nucleotides, such as 22 to 24 nucleotides, such as 23 nucleotides.

As used herein, the term “CRISPR/Cas complex” refers to a ribonucleoprotein complex formed by the binding of a guide RNA or mature crRNA to a Cas protein, which comprises a guide sequence that hybridizes to a target sequence and binds to the Cas protein. The ribonucleoprotein complex is capable of recognizing and cleaving a polynucleotide capable of hybridizing to the guide RNA or mature crRNA.

Therefore, in the case of forming a CRISPR/Cas complex, a “target sequence” refers to a polynucleotide targeted by a guide sequence designed to having targeting ability, such as a sequence complementary to the guide sequence, wherein the hybridization between the target sequence and the guide sequence will promote the formation of the CRISPR/Cas complex. Complete complementarity is not required, as long as there is sufficient complementarity to cause the hybridization and promote the formation of the CRISPR/Cas complex. The target sequence may comprise any polynucleotide, such as DNA or RNA. In some cases, the target sequence is located in the nucleus or cytoplasm of a cell. In some cases, the target sequence may be located in an organelle of a eukaryotic cell, such as a mitochondria or a chloroplast. A sequence or template that can be used for a recombination into a target locus containing the target sequence is referred to as an “editing template” or “editing polynucleotide” or “editing sequence”. In certain embodiments, the editing template is an exogenous nucleic acid. In certain embodiments, the recombination is a homologous recombination.

In the present invention, the expression “target sequence” or “target polynucleotide” can be any polynucleotide that is endogenous or exogenous to a cell (e.g., a eukaryotic cell). For example, the target polynucleotide can be a polynucleotide present in the nucleus of a eukaryotic cell. The target polynucleotide can be a sequence encoding a gene product (e.g., a protein) or a non-coding sequence (e.g., a regulatory polynucleotide or a useless DNA). In some cases, it is believed that the target sequence should be associated with a protospacer adjacent motif (PAM). The exact sequence and length requirements for the PAM vary depending on the Cas effector enzyme used, but the PAM is typically a sequence of 2 to 5 base pairs adjacent to the protospacer sequence (i.e., the target sequence). Those skilled in the art are able to identify the PAM sequence for use together with a given Cas effector protein. Herein, “specific motif sequence recognized by Cas protein” or “motif sequence” refers to a PAM sequence.

In some cases, the target sequence or target polynucleotide may comprise multiple disease-related genes and polynucleotides and signal transduction biochemical pathway-related genes and polynucleotides. Non-limiting examples of such target sequences or target polynucleotides include those listed in U.S. provisional patent applications 61/736,527 and 61/748,427 filed on Dec. 12, 2012 and Jan. 2, 2013, respectively, and international application PCT/US2013/074667 filed on Dec. 12, 2013, all of which are incorporated herein by reference.

In some cases, examples of target sequences or target polynucleotides include sequences related to signal transduction biochemical pathways, such as genes or polynucleotides related to signal transduction biochemical pathways. Examples of target polynucleotides include disease-related genes or polynucleotides. A “disease-associated” gene or polynucleotide refers to any gene or polynucleotide that produces a transcriptional or translational product at an abnormal level or in an abnormal form in cells derived from a disease-affected tissue compared to tissues or cells of a non-disease control. In cases where the altered expression is associated with the onset and/or progression of a disease, it may be a gene that is expressed at an abnormally high level; alternatively, it may be a gene that is expressed at an abnormally low level. A disease-associated gene also refers to a gene that has one or more mutations or genetic variations that are directly responsible for or in linkage disequilibrium with one or more genes responsible for the etiology of the disease. The transcribed or translated product may be known or unknown and may be at normal level or abnormal level.

As used herein, the term “wild type” has the meaning commonly understood by those skilled in the art, which refers to a typical form of an organism, strain, gene, or a characteristic that distinguishes it from a mutant or variant form when it exists in nature, which can be isolated from a source in nature and has not been intentionally and artificially modified.

As used herein, the terms “non-naturally occurring” or “engineered” are used interchangeably and indicate artificial participation. When these terms are used to describe a nucleic acid molecule or polypeptide, it means that the nucleic acid molecule or polypeptide is at least substantially free from at least another component with which it is associated in nature or found in nature.

As used herein, the term “orthologue” or “ortholog” has the meaning commonly understood by those skilled in the art. As a further guide, an “ortholog” of protein as described herein refers to a protein belonging to a different species, and the protein performs the same or similar function as a protein that is its ortholog.

As used herein, the term “identity” is used to refer to the matching of sequences between two polypeptides or between two nucleic acids. When a position in both compared sequences is occupied by the same base or amino acid monomer subunit (e.g., a position in each of the two DNA molecules is occupied by adenine, or a position in each of the two polypeptides is occupied by lysine), then the molecules are identical at that position. The “percentage identity” between two sequences is a function of the number of matching positions shared by the two sequences divided by the number of positions compared×100. For example, if 6 out of 10 positions of the two sequences match, then the two sequences have an identity of 60%. For example, the DNA sequences CTGACT and CAGGTT have an identity of 50% (3 out of a total of 6 positions match). Typically, the two sequences are compared when they are aligned to produce maximum identity. Such an alignment can be achieved by using, for example, the method of Needleman et al. J Mol. Biol. 48: 443 to 453 (1970), which can be conveniently performed by a computer program such as the Align program (DNAstar, Inc.). The algorithm of E. Meyers and W. Miller (Comput. Appl Biosci., 4: 11 to 17 (1988)), which has been incorporated into the ALIGN program (version 2.0), can also be used to determine the percentage identity between two amino acid sequences using a PAM120 weight residue table, a gap length penalty of 12, and a gap penalty of 4. In addition, the percentage identity between two amino acid sequences can be determined using the algorithm of Needleman and Wunsch (J MoI Biol. 48:444 to 453 (1970)), which has been incorporated into the GAP program of the GCG software package (available at www.gcg.com), using a Blossum 62 matrix or a PAM250 matrix as well as a gap weight of 16, 14, 12, 10, 8, 6 or 4 and a length weight of 1, 2, 3, 4, 5 or 6.

As used herein, the term “vector” refers to a nucleic acid delivery vehicle into which a polynucleotide can be inserted. When a vector is capable of expressing a protein encoded by the inserted polynucleotide, the vector is called an expression vector. The vector can be introduced into a host cell by transformation, transduction or transfection so that the genetic material elements it carries are expressed in the host cell. Vectors are well known to those skilled in the art, including but not limited to: plasmid; phagemid; cosmid; artificial chromosome, such as yeast artificial chromosome (YAC), bacterial artificial chromosome (BAC) or P1-derived artificial chromosome (PAC); bacteriophage such as k phage or M13 phage and animal virus, etc. Animal viruses that can be used as vectors include but are not limited to retrovirus (including lentivirus), adenovirus, adeno-associated virus, herpes virus (e.g., herpes simplex virus), poxvirus, baculovirus, papillomavirus, papovavirus (e.g., SV40). A vector may contain a variety of elements for controlling expression, including but not limited to promoter sequence, transcription start sequence, enhancer sequence, selection element and reporter gene. In addition, the vector may also comprise a replication origin.

As used herein, the term “host cell” refers to a cell that can be used to introduce a vector, including but not limited to prokaryotic cell such as Escherichia coli or Bacillus subtilis, fungal cell such as yeast cell or Aspergillus, insect cell such as S2 Drosophila cell or Sf9, or animal cell such as fibroblast, CHO cell, COS cell, NSO cell, HeLa cell, BHK cell, HEK 293 cell or human cell.

Those skilled in the art will understand that the design of expression vector may depend on factors such as the choice of host cell to be transformed, the desired expression level, etc. A vector can be introduced into a host cell to produce a transcript, protein, or peptide, including the protein, fusion protein, isolated nucleic acid molecule, etc. as described herein (e.g., CRISPR transcript, such as nucleic acid transcript, protein, or enzyme).

As used herein, the term “regulatory element” is intended to include a promoter, enhancer, internal ribosome entry site (IRES), and other expression control element (e.g., transcription termination signal, such as polyadenylation signal and poly-U sequence), which may be referred in details to Goeddel, GENE EXPRESSION TECHNOLOGY: METHODS IN ENZYMOLOGY 185, Academic Press, San Diego, California (1990). In some cases, regulatory elements include those sequences that direct the constitutive expression of a nucleotide sequence in many types of host cells and those sequences (e.g., tissue-specific regulatory sequences) that direct the nucleotide sequence to be expressed only in certain host cells. Tissue-specific promoters may primarily direct expression in the desired tissue of interest, such as muscle, neuron, bone, skin, blood, specific organ (e.g., liver, pancreas), or special cell type (e.g., lymphocyte). In some cases, regulatory elements may also direct expression in a timing-dependent manner (e.g., in a cell cycle-dependent or developmental stage-dependent manner), which may or may not be tissue or cell type-specific. In some cases, the term “regulatory element” encompasses enhancer elements, such as WPRE; CMV enhancer; R-U5′ fragment (Mol. Cell. Biol., Vol. 8(1), pp. 466 to 472, 1988); SV40 enhancer; and intron sequence between exons 2 and 3 of rabbit β-globin (Proc. Natl. Acad. Sci. USA., Vol. 78(3), pp. 1527 to 31, 1981).

As used herein, the term “promoter” has the meaning well known to those skilled in the art, which refers to a non-coding nucleotide sequence located upstream of a gene that can initiate expression of a downstream gene. A constitutive promoter is a nucleotide sequence that, when operably linked to a polynucleotide encoding or defining a gene product, leads to the production of a gene product in a cell under most or all of the physiological conditions of the cell. An inducible promoter is a nucleotide sequence that, when operably linked to a polynucleotide encoding or defining a gene product, leads to the production of a gene product in a cell substantially only when an inducer corresponding to the promoter is present in the cell. A tissue-specific promoter is a nucleotide sequence that, when operably linked to a polynucleotide encoding or defining a gene product, leads to the production of a gene product in a cell substantially only when the cell is a cell of the tissue type corresponding to the promoter.

As used herein, the term “operably linked” is intended to mean that the nucleotide sequence of interest is linked to the one or more regulatory elements in a manner that allows the expression of the nucleotide sequence (e.g., in an in vitro transcription/translation system or in a host cell when the vector is introduced into the host cell).

As used herein, the term “complementarity” refers to the ability of a nucleic acid to form one or more hydrogen bonds with another nucleic acid sequence by means of traditional Watson-Crick or other non-traditional types. The percentage of complementarity indicates the percentage of residues in a nucleic acid molecule that can form hydrogen bonds (e.g., Watson-Crick base pairing) with a second nucleic acid sequence (e.g., for 5, 6, 7, 8, 9, 10 out of 10, the complementarity is 50%, 60%, 70%, 80%, 90%, and 100%, respectively). “Complete complementarity” means that all consecutive residues of a nucleic acid sequence form hydrogen bonds with the same number of residues in a second nucleic acid sequence. As used herein, “substantially complementary” refers to a degree of complementarity of at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50 or more nucleotides, or refers to two nucleic acids that hybridize under stringent conditions.

As used herein, “stringent conditions” for hybridization refer to conditions in which a nucleic acid having complementarity to a target sequence predominantly hybridizes to the target sequence and does not substantially hybridize to non-target sequences. Stringent conditions are typically sequence-dependent and vary depending on many factors. In general, the longer the sequence, the higher the temperature at which the sequence specifically hybridizes to its target sequence. Non-limiting examples of stringent conditions are described in Tijssen, Laboratory Techniques In Biochemistry And Molecular Biology-Hybridization With Nucleic Acid Probes, Part I, Chapter 2, “Overview of principles of hybridization and the strategy of nucleic acid probe assay”, (1993), Elsevier, New York.

As used herein, the term “hybridization” refers to a reaction in which one or more polynucleotides react to form a complex that is stabilized by hydrogen bonding of the bases between the nucleotide residues. Hydrogen bonding may occur by means of Watson-Crick base pairing, Hoogstein binding, or in any other sequence-specific manner. The complex may contain two strands forming a duplex, three or more strands forming a multi-stranded complex, a single self-hybridizing strand, or any combination thereof. A hybridization reaction may constitute a step in a broader process (e.g., initiation of PCR, or cleavage of polynucleotide by enzyme). A sequence capable of hybridizing to a given sequence is called the “complement” of the given sequence.

As used herein, the term “expression” refers to a process by which a polynucleotide is transcribed from a DNA template (e.g., transcribed into mRNA or other RNA transcript) and/or a process by which the transcribed mRNA is subsequently translated into a peptide, polypeptide or protein. The transcript and encoded polypeptide can be collectively referred to as “gene products”. If the polynucleotide is derived from a genomic DNA, the expression may comprise splicing of mRNA in a eukaryotic cell.

As used herein, the term “linker” refers to a linear polypeptide formed by multiple amino acid residues connected by peptide bonds. The linker of the present invention can be an artificially synthesized amino acid sequence, or a naturally occurring polypeptide sequence, such as a polypeptide having a hinge region function. Such linker polypeptides are well known in the art (see, for example, Holliger, P. et al. (1993) Proc. Natl. Acad. Sci. USA 90: 6444 to 6448; Poljak, R. J. et al. (1994) Structure 2: 1121-1123).

As used herein, the term “treatment” refers to treating or curing a condition, delaying the onset of symptoms of a condition, and/or delaying the development of a condition.

As used herein, the term “subject” includes, but is not limited to, various animals, such as mammals, such as bovines, equines, ovines, porcines, canines, felines, lagomorphs, rodents (e.g., mice or rats), non-human primates (e.g., macaques or cynomolgus monkeys), or humans. In certain embodiments, the subject (e.g., a human) suffers from a condition (e.g., a condition caused by a disease-related gene defect).

### Beneficial Effects of the Invention

Compared with the prior art, the Cas protein and system of the present invention have significant advantages. For example, the Cas effector protein of the present invention is smaller in molecular size than Cas9, C2c1, CasY, and Cpf1 proteins, and therefore has better transfection efficiency than Cas9, C2c1, CasY, and Cpf1 proteins. For example, the PAM domain of the Cas effector protein of the present invention is of the 5′ RYR structure (R is A or G, Y is T or C), and there are 8 recognizable PAM combinations, namely ATG, ACG, ATA, ACA, GTG, GTA, GCG and GCA, which have wider recognition sites than the currently reported NGG PAM recognized by SpCas9 and TTN PAM recognized by LbCas12a.

The embodiments of the present invention will be described in detail below in conjunction with the accompanying drawings and examples, but those skilled in the art will understand that the following drawings and examples are only used to illustrate the present invention, rather than to limit the scope of the present invention. According to the following detailed description of the accompanying drawings and preferred embodiments, the various objects and advantages of the present invention will become apparent to those skilled in the art.

### BRIEF DESCRIPTION OF THE DRAWINGS

FIGS. 1A and B show the PAM structure analysis and verification results of Casδ-1 in Example 3.

FIG. 2 shows the verification of the PAM recognition site of Casδ-1 in Escherichia coli in Example 3.

FIG. 3 shows the analysis of the cleavage characteristics of Casδ-1 protein on the target sequence in Example 3.

FIGS. 4A and B show the analysis results of the trans-cleavage activity of Casδ-1 in Example 4.

FIG. 5 shows the analysis of the editing activity of Casδ-1 at 14 target sites in HELA cells in Example 5.

FIGS. 6A and B show the editing activity of Casδ-1 on AAVS1 targets at different PAM sites in HELA cells in Example 5.

FIG. 7 shows the editing activity of Casδ-1 for crRNAs composed of direct repeat sequences and guide sequences with different lengths in HELA cells in Example 5, in which R represents direct repeat sequences, T represents guide sequences, and the numbers represent the number of bases in the corresponding sequence.

FIG. 8A shows the components in the expression vector with the protein of Casδ-1 constructed in Example 6; FIG. 8B shows the base sequences of Casδ-1-edited plants in maize in Example 6.

FIG. 9 shows the cleavage results of Casδ-1D31 on double-stranded DNA in Example 9.

### SEQUENCE INFORMATION

The information of the partial sequences involved in the present invention is provided in Table 1 below.

TABLE 1
Description of sequences

| SEQ | |
|---|---|
| ID | |
| NO: | Description |
| 1 | Amino acid sequence of Casδ-1 |
| | MSKDGDKMKHSKQEVSEDKGVKKDPSVFRCQNLAILEMREEVADYYADLQADYR |
| | HYLPLMILWGQNGFMEPDETGLNFSAVLPSTAKTRCENRYGAQIAADLKLIADDLP |
| | SSLYAIFTNDYQPLTGVKKVGDRWKFVTSLNTVSDEEFAKKVKPLLRDYKGVTEEQ |
| | WRRFREER VAGKTADQVWDDLDSILRNPTLAALKKIHDKHMFRVKRPESKLYCDA |
| | LAHmLSSSYDSWKKINEARQLELQAMRDEQQGLLANKTTNSLYNLVCEFADELESQ |
| | NYGLTRRIFFIARKEWEDEKSHYLPVIKLLVTEKYLPLLQADNKTVSPAVKAWDICS |
| | TLKQRSPYIRFVPPTKDYPIPFGQTGRGHKFTLKEEAGKVCFLVPGLTKDEMPLSGSH |
| | YFSSLRIDKVGTDFKISFRHKTKIRSKKSARVTVKQPRINGTVTEVGILRRNGKFFLR |
| | VAYKIAYPQHNIDLKSYFASAAPSTQKLELLPAQMRVAGVDLNISDPIVVSKADIFK |
| | GEDGGPLSVLDYGSGKVVGEPVIICKDTSRAGKISSLATKCRTLRDVIRNFRHSLHQ |
| | GTPLPSKSLEFLQEVPVAQEDLKSPSPRYLIQTWIKYIRTEMKRLHYKVWLDGYCHT |
| | SEAMRmLDLVDQVAFLMKSYENIHVKPGHKIPKKTTASLKIRETSRTRFRLHVSRLV |
| | GARVVRACEDCQMIFLEDLSTGFSEGSNNSLARLFSAGQLKNSIVMAAEKVGKAVV |
| | FVCKDGTSIKDPVFGLHGLRPSKKTPGIPPDKKRLYVRRDGKIGYINADQAASLNVL |
| | LVGLSHSVAKYKFFVKDGKLQDDEEVAAQQGKKPRKIGVRLERLLKMNFDTPARL |
| | FFSLEKDQVVPCYEATETPYTGWVYVYHDRLLTTRQRDQQVNQLAQEVKDLLRDG |
| | VKIPKWDVIPDCDNSCLGFSPCLVLDDETAAVSVV |
| 2 | Amino acid sequence of Casδ-2 |
| | MELKTTFCQSLKIKILPESHKLYYDELQADLKKMRIFFATHSGLNYSYKEDENDSEW |
| | KIASSKKSTLSPEAQRLIDIIAKGGIGAEHAYGIFYKDKKVSDKIRRDNSNCIFAmLLP |
| | NLVNFEEEYKKRKSKLQLTKEEWEEYHKRCLAGESVEVLWSEEIAPKIGPNEAARL |
| | ASLAYHQFPDSRHPADITHCRALMSMVADSFCSWVECHKLHREETEKLKTKLQEKI |
| | ESCKEAYNLLLAYSEDLYSRNYGLSARVLKALIDNKSRCCFDIAFQIAEKYPALMKI |
| | DEKTLFKAYNALKLHWKIKRRKPFISLPKIERDYQVPFGLTGSRGKKFDVYVENKNI |
| | VVEIDGQKVETFSSHYFSDMQISKIYGEKKNLKGFKLKFRHKLKDKKKEVYGEWIE |
| | AELKEIKIKKDMETGDFYLYLPYTTTHNEKNLLLEKFFSYADPLKETIFKNGKTELPN |
| | EFLGFGFDLNLSDPIAMAVAEFVRDSSDGEIGALDYGHGKLLDASVLVCNSSLSKRI |
| | NDLVGDNRKLIQAIRSYKNSLVTKEMDEESGEWLKKALGNAKYGNHRHQLQLVMS |
| | KLNKKSKKYWQECRKNGHNDLSENIALLKLLDVQFSLHKSYNNIHIFDYKKQIHSH |
| | KTDSKRENFREFVTKQFAATIVKHCKEVMAKYGREYAVVFLEDLEMYFDADADNN |
| | SLIRLFAPGQLKKYIASALAKSKIGYVFIPPAGTSKTDPLTSKIGFRATGKYYKQLGY |
| | LLNKSDLYVERNGVIGKINSDVAAAINILLKGVNHSIVPYRFLNKHAGSEQKRLKRF |
| | EGEIGCDLKKMPKTRLYYLDGKIITEEEKNSLEEALLAEIKHFLLSKANIPEFDAIPGS |
| | NGTCKAFSVPRCLK |
| 3 | Amino acid sequence of Casδ-1D31 |
| | NLAILEMREEVADYYADLQADYRHYLPLMILWGQNGFMEPDETGLNFSAVLPSTA |
| | KTRCENRYGAQIAADLKLIADDLPSSLYAIFTNDYQPLTGVKKVGDRWKFVTSLNT |
| | VSDEEFAKKVKPLLRDYKGVTEEQWRRFREERVAGKTADQVWDDLDSILRNPTLA |
| | ALKKIHDKHMFRVKRPESKLYCDALAHmLSSSYDSWKKINEARQLELQAMRDEQQ |
| | GLLANKTTNSLYNLVCEFADELESQNYGLTRRIFFIARKEWEDEKSHYLPVIKLLVT |
| | EKYLPLLQADNKTVSPAVKAWDICSTLKQRSPYIRFVPPTKDYPIPFGQTGRGHKFT |
| | LKEEAGKVCFLVPGLTKDEMPLSGSHYFSSLRIDKVGTDFKISFRHKTKIRSKKSARV |
| | TVKQPRINGTVTEVGILRRNGKFFLRVAYKIAYPQHNIDLKSYFASAAPSTQKLELLP |
| | AQMRVAGVDLNISDPIVVSKADIFKGEDGGPLSVLDYGSGKVVGEPVIICKDTSRAG |
| | KISSLATKCRTLRDVIRNFRHSLHQGTPLPSKSLEFLQEVPVAQEDLKSPSPRYLIQT |
| | WIKYIRTEMKRLHYKVWLDGYCHTSEAMRmLDLVDQVAFLMKSYENIHVKPGHKI |
| | PKKTTASLKIRETSRTRFRLHVSRLVGARVVRACEDCQMIFLEDLSTGFSEGSNNSLA |
| | RLFSAGQLKNSIVMAAEKVGKAVVFVCKDGTSIKDPVFGLHGLRPSKKTPGIPPDKK |
| | RLYVRRDGKIGYINADQAASLNVLLVGLSHSVAKYKFFVKDGKLQDDEEVAAQQG |
| | KKPRKIGVRLERLLKMNFDTPARLFFSLEKDQVVPCYEATETPYTGWVYVYHDRLL |
| | TTRQRDQQVNQLAQEVKDLLRDGVKIPKWDVIPDCDNSCLGFSPCLVLDDETAAVS |
| | VV |
| 4 | Encoding nucleotide sequence of Casδ-1 |
| | ATGTCCAAAGACGGTGACAAGATGAAGCACAGCAAACAAGAAGTCTCTGAGGA |
| | CAAAGGGGTCAAGAAAGACCCGTCTGTCTTCCGCTGTCAGAACCTCGCGATTCT |
| | GGAGATGCGTGAAGAAGTCGCTGATTACTACGCCGACCTTCAGGCCGACTACCG |
| | TCACTACTTGCCCCTGATGATCCTCTGGGGCCAGAACGGGTTCATGGAGCCAGA |
| | CGAAACTGGTCTGAACTTCAGTGCCGTCCTGCCATCGACCGCAAAAACACGGTG |
| | CGAGAACCGCTACGGTGCGCAGATTGCGGCTGATCTCAAGTTGATCGCCGACGA |
| | TCTCCCGAGCAGTCTCTATGCCATCTTCACCAACGACTACCAGCCCCTCACCGGC |
| | GTGAAGAAGGTCGGCGACAGATGGAAGTTCGTCACGTCCCTCAACACTGTCAGC |
| | GACGAAGAGTTCGCCAAGAAGGTGAAGCCGCTCCTGCGTGACTACAAAGGCGTT |
| | ACCGAAGAGCAGTGGCGTCGATTTCGTGAAGAGCGTGTCGCCGGCAAGACAGCC |
| | GATCAGGTCTGGGACGACCTTGACAGCATTCTGCGAAACCCGACGCTCGCGGCC |
| | CTGAAGAAGATCCACGACAAGCACATGTTCCGGGTCAAGCGACCCGAGAGCAA |
| | ACTCTACTGTGACGCCCTGGCTCACATGCTGAGTTCCAGCTACGACAGTTGGAA |
| | GAAAATCAACGAGGCCCGCCAACTCGAACTCCAAGCCATGCGTGACGAGCAGC |
| | AGGGCTTGCTCGCCAACAAGACCACGAACAGTCTGTACAATCTGGTGTGCGAGT |
| | TCGCCGACGAACTGGAGTCGCAGAACTACGGTCTGACTCGGCGGATCTTCTTCA |
| | TCGCCCGCAAAGAGTGGGAGGACGAAAAGTCCCATTACCTCCCGGTCATCAAGC |
| | TCCTCGTCACGGAGAAGTATCTGCCCCTGCTCCAGGCGGATAACAAGACTGTCA |
| | GCCCCGCCGTCAAGGCGTGGGACATCTGCTCTACTCTGAAGCAGAGGTCGCCGT |
| | ACATCCGCTTCGTGCCTCCGACCAAGGACTATCCGATCCCCTTCGGCCAGACGG |
| | GTCGCGGACACAAGTTCACCTTGAAAGAGGAAGCCGGAAAGGTCTGTTTCCTCG |
| | TACCCGGCCTGACCAAGGACGAGATGCCTCTGTCCGGCTCTCACTACTTCTCAAG |
| | CCTCCGTATCGACAAGGTCGGCACAGACTTCAAGATCTCGTTCCGTCACAAGAC |
| | CAAGATTCGCAGCAAAAAGTCTGCGCGAGTCACCGTCAAGCAGCCCAGGATCAA |
| | CGGCACCGTCACTGAGGTCGGCATCCTACGCAGGAACGGCAAGTTCTTCCTGCG |
| | TGTCGCTTACAAGATCGCCTACCCACAGCACAACATCGACCTGAAGAGCTACTT |
| | CGCCTCGGCAGCCCCCTCCACGCAAAAGCTGGAGCTTCTGCCGGCTCAAATGAG |
| | AGTCGCGGGCGTCGATCTGAACATCAGCGACCCGATTGTCGTCTCCAAGGCCGA |
| | CATCTTCAAGGGCGAAGACGGCGGGCCGCTGTCTGTGCTGGACTACGGCTCTGG |
| | CAAGGTGGTAGGTGAGCCTGTGATCATCTGCAAGGACACCAGCAGGGCCGGGA |
| | AGATCAGTTCCCTCGCCACGAAATGCCGGACGCTACGCGACGTGATCCGCAACT |
| | TTCGTCATTCACTGCATCAGGGTACGCCCTTGCCCAGCAAGAGTCTTGAATTTCT |
| | CCAAGAAGTTCCCGTGGCGCAGGAGGACTTGAAGTCGCCCAGCCCTCGCTATCT |
| | GATCCAGACGTGGATCAAGTACATCAGGACCGAGATGAAGCGACTTCACTACAA |
| | GGTCTGGCTCGACGGGTACTGCCACACTAGCGAAGCCATGCGGATGCTCGATCT |
| | GGTCGATCAGGTCGCGTTCCTAATGAAGTCGTATGAGAACATCCACGTCAAACC |
| | GGGTCACAAGATCCCGAAGAAGACCACTGCGTCCCTGAAGATTCGCGAGACGAG |
| | CCGAACGCGGTTCAGGCTGCATGTCTCCCGACTTGTCGGTGCGCGAGTGGTTCGT |
| | GCGTGCGAGGACTGCCAGATGATCTTCTTGGAAGATCTCTCGACAGGTTTCTCCG |
| | AAGGCAGCAATAACTCGTTGGCCCGGCTGTTTTCGGCTGGCCAACTGAAGAACT |
| | CCATCGTCATGGCGGCAGAGAAGGTCGGCAAAGCCGTAGTCTTCGTGTGCAAGG |
| | ACGGTACGTCTATCAAAGATCCTGTTTTCGGTCTGCACGGCCTGCGGCCTTCCAA |
| | GAAGACCCCAGGCATTCCGCCGGACAAGAAGCGTCTCTACGTCCGTCGCGACGG |
| | CAAAATCGGCTACATCAACGCCGATCAGGCGGCTTCGCTGAATGTGCTGCTGGT |
| | GGGGCTGTCGCACTCCGTCGCCAAGTACAAGTTCTTTGTCAAGGACGGGAAGCT |
| | ACAAGACGACGAGGAGGTGGCTGCCCAGCAAGGCAAGAAGCCGCGTAAGATCG |
| | GTGTTCGTCTGGAAAGACTCCTCAAGATGAACTTCGACACCCCTGCCAGACTTTT |
| | CTTCTCGCTGGAGAAAGATCAGGTGGTGCCGTGCTACGAGGCCACGGAAACGCC |
| | TTACACCGGCTGGGTCTACGTCTACCACGACCGACTGCTGACCACCCGGCAACG |
| | TGATCAGCAAGTCAATCAACTCGCACAGGAAGTCAAGGATCTCCTGCGGGATGG |
| | TGTCAAAATTCCTAAATGGGACGTAATCCCAGACTGCGACAACAGTTGCCTCGG |
| | CTTCTCTCCGTGTCTGGTGCTTGACGACGAAACCGCCGCTGTTTCTGTGGTTTAG |
| 5 | Encoding nucleotide sequence of Casδ-2 |
| | ATGGAATTGAAGACAACTTTTTGTCAGTCACTAAAAATTAAGATCCTCCCCGAA |
| | AGCCATAAGTTGTATTACGATGAGCTGCAAGCCGACCTAAAGAAGATGAGAATC |
| | TTCTTCGCAACGCACTCTGGGCTCAACTACAGCTACAAGGAAGATGAAAACGAT |
| | TCGGAATGGAAGATAGCGTCGTCAAAAAAGAGCACACTCTCACCTGAAGCCCAA |
| | AGGCTTATTGACATCATCGCCAAAGGCGGTATAGGAGCAGAGCACGCCTACGGA |
| | ATATTTTACAAGGACAAAAAGGTGTCGGACAAGATCCGGCGAGATAATTCAAAC |
| | TGCATCTTCGCCATGCTATTACCTAACCTCGTCAATTTCGAGGAAGAATACAAAA |
| | AGAGGAAAAGTAAGCTCCAGCTCACCAAGGAAGAGTGGGAGGAGTACCATAAA |
| | CGATGTCTTGCAGGCGAGAGCGTGGAAGTCCTGTGGTCCGAGGAGATTGCTCCA |
| | AAAATTGGGCCTAACGAAGCGGCGAGACTGGCATCACTCGCGTATCACCAGTTC |
| | CCAGATTCTCGTCATCCGGCGGACATTACCCACTGCAGGGCGCTCATGTCTATGG |
| | TAGCAGACTCTTTTTGTTCCTGGGTCGAGTGCCACAAGCTGCACCGTGAGGAAA |
| | CAGAGAAACTCAAGACGAAATTACAGGAGAAGATAGAGAGCTGCAAGGAGGCA |
| | TACAATCTGCTACTCGCTTATTCCGAAGACCTGTACAGTAGGAATTATGGTCTGA |
| | GTGCCAGGGTTTTGAAGGCACTTATTGACAACAAATCCCGGTGCTGCTTCGACAT |
| | AGCCTTTCAGATCGCGGAGAAATATCCTGCACTGATGAAGATTGATGAAAAGAC |
| | CCTGTTCAAGGCCTACAACGCCCTAAAGCTCCACTGGAAAATAAAACGACGCAA |
| | GCCGTTCATCTCGCTGCCCAAGATCGAGAGGGACTACCAGGTGCCCTTTGGACT |
| | CACAGGAAGCAGAGGCAAGAAGTTCGACGTCTATGTGGAAAACAAGAATATTG |
| | TGGTCGAGATCGACGGCCAGAAAGTCGAAACCTTCAGCTCTCACTATTTCTCTGA |
| | TATGCAGATCTCCAAGATTTACGGTGAAAAGAAAAATTTGAAGGGATTCAAGTT |
| | GAAATTCCGCCACAAGCTGAAGGACAAGAAGAAGGAGGTTTATGGAGAGTGGA |
| | TTGAGGCGGAGCTGAAGGAAATCAAGATTAAGAAAGACATGGAGACAGGCGAC |
| | TTCTACCTGTATCTCCCATACACTACAACGCATAATGAGAAAAACTTATTGCTCG |
| | AGAAGTTCTTTTCCTACGCCGATCCACTTAAGGAGACTATTTTTAAGAACGGAAA |
| | GACGGAGCTTCCAAATGAATTTCTAGGCTTCGGCTTCGATTTGAACCTTTCCGAC |
| | CCGATTGCGATGGCGGTTGCCGAGTTTGTTCGGGACTCATCAGATGGTGAGATT |
| | GGCGCCCTCGACTATGGTCACGGTAAGCTCCTCGACGCTAGTGTTTTGGTCTGCA |
| | ACTCATCACTATCAAAGCGCATCAATGACCTGGTTGGAGACAATAGAAAGCTGA |
| | TCCAAGCTATTAGATCGTACAAGAACTCGCTAGTAACTAAGGAAATGGACGAGG |
| | AGAGTGGCGAGTGGCTTAAAAAAGCTCTAGGCAATGCAAAGTACGGCAACCAT |
| | AGGCATCAGCTTCAACTGGTCATGTCTAAGCTGAACAAAAAAAGCAAAAAGTAC |
| | TGGCAGGAATGTCGCAAGAACGGCCACAACGATCTTTCCGAAAATATTGCACTA |
| | TTAAAGTTGTTGGACGTGCAGTTCTCTTTGCATAAAAGCTATAACAACATACACA |
| | TCTTTGATTACAAAAAACAAATTCACAGCCATAAGACTGACTCCAAAAGGGAGA |
| | ATTTCCGTGAATTTGTGACGAAGCAATTTGCGGCTACCATAGTAAAGCATTGCA |
| | AGGAGGTGATGGCTAAATATGGCAGAGAGTACGCCGTTGTCTTTTTGGAGGACC |
| | TGGAGATGTACTTTGATGCTGATGCTGATAACAATTCTCTGATCCGGCTTTTTGC |
| | ACCCGGCCAATTGAAGAAGTACATCGCCTCCGCTCTGGCTAAGAGCAAGATAGG |
| | TTATGTGTTCATCCCTCCTGCTGGGACAAGTAAGACCGACCCGTTAACTTCGAAA |
| | ATTGGATTCAGGGCTACCGGAAAATACTACAAGCAGCTGGGCTATCTGCTTAAT |
| | AAGTCCGACCTCTACGTGGAGCGCAACGGGGTTATCGGGAAGATCAACAGTGAT |
| | GTAGCTGCGGCGATAAATATTCTCTTAAAGGGCGTGAACCACTCAATCGTGCCG |
| | TACCGCTTCCTGAATAAACATGCTGGGTCTGAACAGAAGCGCCTCAAACGTTTT |
| | GAAGGGGAGATCGGTTGTGATCTGAAAAAGATGCCAAAGACACGCCTCTACTAT |
| | CTTGACGGAAAAATCATCACTGAAGAGGAGAAGAACAGCCTGGAGGAAGCGCT |
| | ACTCGCTGAGATCAAGCACTTCCTTCTGTCGAAGGCCAACATTCCGGAGTTTGAT |
| | GCAATACCGGGGAGCAATGGCACCTGCAAAGCCTTCTCCGTTCCACGCTGCCTT |
| | AAGTGA |
| 6 | Encoding nucleotide sequence of Casδ-1D31 |
| | AACCTCGCGATTCTGGAGATGCGTGAAGAAGTCGCTGATTACTACGCCGACCTT |
| | CAGGCCGACTACCGTCACTACTTGCCCCTGATGATCCTCTGGGGCCAGAACGGG |
| | TTCATGGAGCCAGACGAAACTGGTCTGAACTTCAGTGCCGTCCTGCCATCGACC |
| | GCAAAAACACGGTGCGAGAACCGCTACGGTGCGCAGATTGCGGCTGATCTCAAG |
| | TTGATCGCCGACGATCTCCCGAGCAGTCTCTATGCCATCTTCACCAACGACTACC |
| | AGCCCCTCACCGGCGTGAAGAAGGTCGGCGACAGATGGAAGTTCGTCACGTCCC |
| | TCAACACTGTCAGCGACGAAGAGTTCGCCAAGAAGGTGAAGCCGCTCCTGCGTG |
| | ACTACAAAGGCGTTACCGAAGAGCAGTGGCGTCGATTTCGTGAAGAGCGTGTCG |
| | CCGGCAAGACAGCCGATCAGGTCTGGGACGACCTTGACAGCATTCTGCGAAACC |
| | CGACGCTCGCGGCCCTGAAGAAGATCCACGACAAGCACATGTTCCGGGTCAAGC |
| | GACCCGAGAGCAAACTCTACTGTGACGCCCTGGCTCACATGCTGAGTTCCAGCT |
| | ACGACAGTTGGAAGAAAATCAACGAGGCCCGCCAACTCGAACTCCAAGCCATGC |
| | GTGACGAGCAGCAGGGCTTGCTCGCCAACAAGACCACGAACAGTCTGTACAATC |
| | TGGTGTGCGAGTTCGCCGACGAACTGGAGTCGCAGAACTACGGTCTGACTCGGC |
| | GGATCTTCTTCATCGCCCGCAAAGAGTGGGAGGACGAAAAGTCCCATTACCTCC |
| | CGGTCATCAAGCTCCTCGTCACGGAGAAGTATCTGCCCCTGCTCCAGGCGGATA |
| | ACAAGACTGTCAGCCCCGCCGTCAAGGCGTGGGACATCTGCTCTACTCTGAAGC |
| | AGAGGTCGCCGTACATCCGCTTCGTGCCTCCGACCAAGGACTATCCGATCCCCTT |
| | CGGCCAGACGGGTCGCGGACACAAGTTCACCTTGAAAGAGGAAGCCGGAAAGG |
| | TCTGTTTCCTCGTACCCGGCCTGACCAAGGACGAGATGCCTCTGTCCGGCTCTCA |
| | CTACTTCTCAAGCCTCCGTATCGACAAGGTCGGCACAGACTTCAAGATCTCGTTC |
| | CGTCACAAGACCAAGATTCGCAGCAAAAAGTCTGCGCGAGTCACCGTCAAGCAG |
| | CCCAGGATCAACGGCACCGTCACTGAGGTCGGCATCCTACGCAGGAACGGCAAG |
| | TTCTTCCTGCGTGTCGCTTACAAGATCGCCTACCCACAGCACAACATCGACCTGA |
| | AGAGCTACTTCGCCTCGGCAGCCCCCTCCACGCAAAAGCTGGAGCTTCTGCCGG |
| | CTCAAATGAGAGTCGCGGGCGTCGATCTGAACATCAGCGACCCGATTGTCGTCT |
| | CCAAGGCCGACATCTTCAAGGGCGAAGACGGCGGGCCGCTGTCTGTGCTGGACT |
| | ACGGCTCTGGCAAGGTGGTAGGTGAGCCTGTGATCATCTGCAAGGACACCAGCA |
| | GGGCCGGGAAGATCAGTTCCCTCGCCACGAAATGCCGGACGCTACGCGACGTGA |
| | TCCGCAACTTTCGTCATTCACTGCATCAGGGTACGCCCTTGCCCAGCAAGAGTCT |
| | TGAATTTCTCCAAGAAGTTCCCGTGGCGCAGGAGGACTTGAAGTCGCCCAGCCC |
| | TCGCTATCTGATCCAGACGTGGATCAAGTACATCAGGACCGAGATGAAGCGACT |
| | TCACTACAAGGTCTGGCTCGACGGGTACTGCCACACTAGCGAAGCCATGCGGAT |
| | GCTCGATCTGGTCGATCAGGTCGCGTTCCTAATGAAGTCGTATGAGAACATCCA |
| | CGTCAAACCGGGTCACAAGATCCCGAAGAAGACCACTGCGTCCCTGAAGATTCG |
| | CGAGACGAGCCGAACGCGGTTCAGGCTGCATGTCTCCCGACTTGTCGGTGCGCG |
| | AGTGGTTCGTGCGTGCGAGGACTGCCAGATGATCTTCTTGGAAGATCTCTCGAC |
| | AGGTTTCTCCGAAGGCAGCAATAACTCGTTGGCCCGGCTGTTTTCGGCTGGCCAA |
| | CTGAAGAACTCCATCGTCATGGCGGCAGAGAAGGTCGGCAAAGCCGTAGTCTTC |
| | GTGTGCAAGGACGGTACGTCTATCAAAGATCCTGTTTTCGGTCTGCACGGCCTGC |
| | GGCCTTCCAAGAAGACCCCAGGCATTCCGCCGGACAAGAAGCGTCTCTACGTCC |
| | GTCGCGACGGCAAAATCGGCTACATCAACGCCGATCAGGCGGCTTCGCTGAATG |
| | TGCTGCTGGTGGGGCTGTCGCACTCCGTCGCCAAGTACAAGTTCTTTGTCAAGGA |
| | CGGGAAGCTACAAGACGACGAGGAGGTGGCTGCCCAGCAAGGCAAGAAGCCGC |
| | GTAAGATCGGTGTTCGTCTGGAAAGACTCCTCAAGATGAACTTCGACACCCCTG |
| | CCAGACTTTTCTTCTCGCTGGAGAAAGATCAGGTGGTGCCGTGCTACGAGGCCA |
| | CGGAAACGCCTTACACCGGCTGGGTCTACGTCTACCACGACCGACTGCTGACCA |
| | CCCGGCAACGTGATCAGCAAGTCAATCAACTCGCACAGGAAGTCAAGGATCTCC |
| | TGCGGGATGGTGTCAAAATTCCTAAATGGGACGTAATCCCAGACTGCGACAACA |
| | GTTGCCTCGGCTTCTCTCCGTGTCTGGTGCTTGACGACGAAACCGCCGCTGTTTC |
| | TGTGGTTTAG |
| 7 | Direct repeat sequence of Casδ-1 |
| | GUGCUGACGACCAGCACUAGAUGGUCGUUCAGGCAC |
| 8 | Direct repeat sequence of Casδ-2 |
| | GUGCUGAACAGGGUCGCUAGGCGUUGUUCAAGGCAC |
| 9 | Direct repeat sequence of Casδ-1R34 |
| | GCUGACGACCAGCACUAGAUGGUCGUUCAGGCAC |
| 10 | Direct repeat sequence of Casδ-1R32 |
| | UGACGACCAGCACUAGAUGGUCGUUCAGGCAC |
| 11 | Direct repeat sequence of Casδ-1R30 |
| | ACGACCAGCACUAGAUGGUCGUUCAGGCAC |
| 12 | Direct repeat sequence of Casδ-1R28 |
| | GACCAGCACUAGAUGGUCGUUCAGGCAC |
| 13 | Direct repeat sequence of Casδ-1R26 |
| | CCAGCACUAGAUGGUCGUUCAGGCAC |
| 14 | Direct repeat sequence of Casδ-1R24 |
| | AGCACUAGAUGGUCGUUCAGGCAC |
| 15 | Direct repeat sequence of Casδ-1R22 |
| | CACUAGAUGGUCGUUCAGGCAC |
| 16 | Direct repeat sequence of Casδ-1R20 |
| | CUAGAUGGUCGUUCAGGCAC |
| 17 | Encoding nucleic acid sequence of direct repeat sequence of Casδ-1 |
| | GTGCTGACGACCAGCACTAGATGGTCGTTCAGGCAC |
| 18 | Encoding nucleic acid sequence of direct repeat sequence of Casδ-2 |
| | GTGCTGAACAGGGTCGCTAGGCGTTGTTCAAGGCACG |
| 19 | Encoding nucleic acid sequence of direct repeat sequence of Casδ-1R34 |
| | GCTGACGACCAGCACTAGATGGTCGTTCAGGCAC |
| 20 | Encoding nucleic acid sequence of direct repeat sequence of Casδ-1R32 |
| | TGACGACCAGCACTAGATGGTCGTTCAGGCAC |
| 21 | Encoding nucleic acid sequence of direct repeat sequence of Casδ-1R30 |
| | ACGACCAGCACTAGATGGTCGTTCAGGCAC |
| 22 | Encoding nucleic acid sequence of direct repeat sequence of Casδ-1R28 |
| | GACCAGCACTAGATGGTCGTTCAGGCAC |
| 23 | Encoding nucleic acid sequence of direct repeat sequence of Casδ-1R26 |
| | CCAGCACTAGATGGTCGTTCAGGCAC |
| 24 | Encoding nucleic acid sequence of direct repeat sequence of Casδ-1R24 |
| | AGCACTAGATGGTCGTTCAGGCAC |
| 25 | Encoding nucleic acid sequence of direct repeat sequence of Casδ-1R22 |
| | CACTAGATGGTCGTTCAGGCAC |
| 26 | Encoding nucleic acid sequence of direct repeat sequence of Casδ-1R20 |
| | CTAGATGGTCGTTCAGGCAC |
| 27 | NLS sequence |
| | SRADPKKKRKV |
| 28 | Amino acid sequence of Casδ-1-NLS fusion protein |
| | MGPKKKRKVMDYKDHDGDYKDHDIDYKDDDDKMSKDGDKMKHSKQEVSEDKG |
| | VKKDPSVFRCONLAILEMREEVADYYADLQADYRHYLPLMILWGQNGFMEPDETG |
| | LNFSAVLPSTAKTRCENRYGAQIAADLKLIADDLPSSLYAIFTNDYQPLTGVKKVGD |
| | RWKFVTSLNTVSDEEFAKKVKPLLRDYKGVTEEQWRRFREERVAGKTADQVWDD |
| | LDSILRNPTLAALKKIHDKHMFRVKRPESKLYCDALAHmLSSSYDSWKKINEARQL |
| | ELQAMRDEQQGLLANKTTNSLYNLVCEFADELESQNYGLTRRIFFIARKEWEDEKS |
| | HYLPVIKLLVTEKYLPLLQADNKTVSPAVKAWDICSTLKQRSPYIRFVPPTKDYPIPF |
| | GQTGRGHKFTLKEEAGKVCFLVPGLTKDEMPLSGSHYFSSLRIDKVGTDFKISFRHK |
| | TKIRSKKSARVTVKQPRINGTVTEVGILRRNGKFFLRVAYKIAYPQHNIDLKSYFASA |
| | APSTQKLELLPAQMRVAGVDLNISDPIVVSKADIFKGEDGGPLSVLDYGSGKVVGEP |
| | VIICKDTSRAGKISSLATKCRTLRDVIRNFRHSLHQGTPLPSKSLEFLQEVPVAQEDL |
| | KSPSPRYLIQTWIKYIRTEMKRLHYKVWLDGYCHTSEAMRmLDLVDQVAFLMKSY |
| | ENIHVKPGHKIPKKTTASLKIRETSRTRFRLHVSRLVGARVVRACEDCQMIFLEDLST |
| | GFSEGSNNSLARLFSAGQLKNSIVMAAEKVGKAVVFVCKDGTSIKDPVFGLHGLRP |
| | SKKTPGIPPDKKRLYVRRDGKIGYINADQAASLNVLLVGLSHSVAKYKFFVKDGKL |
| | QDDEEVAAQQGKKPRKIGVRLERLLKMNFDTPARLFFSLEKDQVVPCYEATETPYT |
| | GWVYVYHDRLLTTRQRDQQVNQLAQEVKDLLRDGVKIPKWDVIPDCDNSCLGFSP |
| | CLVLDDETAAVSVVSRADPKKKRKV |
| 29 | Amino acid sequence of Casδ-2-NLS fusion protein |
| | MGPKKKRKVMDYKDHDGDYKDHDIDYKDDDDKMELKTTFCQSLKIKILPESHKLY |
| | YDELQADLKKMRIFFATHSGLNYSYKEDENDSEWKIASSKKSTLSPEAQRLIDIIAKG |
| | GIGAEHAYGIFYKDKKVSDKIRRDNSNCIFAmLLPNLVNFEEEYKKRKSKLQLTKEE |
| | WEEYHKRCLAGESVEVLWSEEIAPKIGPNEAARLASLAYHQFPDSRHPADITHCRAL |
| | MSMVADSFCSWVECHKLHREETEKLKTKLQEKIESCKEAYNLLLAYSEDLYSRNYG |
| | LSARVLKALIDNKSRCCFDIAFQIAEKYPALMKIDEKTLFKAYNALKLHWKIKRRKP |
| | FISLPKIERDYQVPFGLTGSRGKKFDVYVENKNIVVEIDGQKVETFSSHYFSDMQISK |
| | IYGEKKNLKGFKLKFRHKLKDKKKEVYGEWIEAELKEIKIKKDMETGDFYLYLPYT |
| | TTHNEKNLLLEKFFSYADPLKETIFKNGKTELPNEFLGFGFDLNLSDPIAMAVAEFVR |
| | DSSDGEIGALDYGHGKLLDASVLVCNSSLSKRINDLVGDNRKLIQAIRSYKNSLVTK |
| | EMDEESGEWLKKALGNAKYGNHRHQLQLVMSKLNKKSKKYWQECRKNGHNDLS |
| | ENIALLKLLDVQFSLHKSYNNIHIFDYKKQIHSHKTDSKRENFREFVTKQFAATIVKH |
| | CKEVMAKYGREYAVVFLEDLEMYFDADADNNSLIRLFAPGQLKKYIASALAKSKIG |
| | YVFIPPAGTSKTDPLTSKIGFRATGKYYKQLGYLLNKSDLYVERNGVIGKINSDVAA |
| | AINILLKGVNHSIVPYRFLNKHAGSEQKRLKRFEGEIGCDLKKMPKTRLYYLDGKIIT |
| | EEEKNSLEEALLAEIKHFLLSKANIPEFDAIPGSNGTCKAFSVPRCLKSRADPKKKRK |
| | V |
| 30 | Amino acid sequence of Casδ-1D31-NLS fusion protein |
| | MGPKKKRKVMDYKDHDGDYKDHDIDYKDDDDKNLAILEMREEVADYYADLQAD |
| | YRHYLPLMILWGQNGFMEPDETGLNFSAVLPSTAKTRCENRYGAQIAADLKLIADD |
| | LPSSLYAIFTNDYQPLTGVKKVGDRWKFVTSLNTVSDEEFAKKVKPLLRDYKGVTE |
| | EQWRRFREERVAGKTADQVWDDLDSILRNPTLAALKKIHDKHMFRVKRPESKLYC |
| | DALAHmLSSSYDSWKKINEARQLELQAMRDEQQGLLANKTTNSLYNLVCEFADEL |
| | ESQNYGLTRRIFFIARKEWEDEKSHYLPVIKLLVTEKYLPLLQADNKTVSPAVKAWD |
| | ICSTLKQRSPYIRFVPPTKDYPIPFGQTGRGHKFTLKEEAGKVCFLVPGLTKDEMPLS |
| | GSHYFSSLRIDKVGTDFKISFRHKTKIRSKKSARVTVKQPRINGTVTEVGILRRNGKF |
| | FLRVAYKIAYPQHNIDLKSYFASAAPSTQKLELLPAQMRVAGVDLNISDPIVVSKADI |
| | FKGEDGGPLSVLDYGSGKVVGEPVIICKDTSRAGKISSLATKCRTLRDVIRNFRHSLH |
| | QGTPLPSKSLEFLQEVPVAQEDLKSPSPRYLIQTWIKYIRTEMKRLHYKVWLDGYCH |
| | TSEAMRmLDLVDQVAFLMKSYENIHVKPGHKIPKKTTASLKIRETSRTRFRLHVSRL |
| | VGARVVRACEDCQMIFLEDLSTGFSEGSNNSLARLFSAGQLKNSIVMAAEKVGKAV |
| | VFVCKDGTSIKDPVFGLHGLRPSKKTPGIPPDKKRLYVRRDGKIGYINADQAASLNV |
| | LLVGLSHSVAKYKFFVKDGKLQDDEEVAAQQGKKPRKIGVRLERLLKMNFDTPAR |
| | LFFSLEKDQVVPCYEATETPYTGWVYVYHDRLLTTRQRDQQVNQLAQEVKDLLRD |
| | GVKIPKWDVIPDCDNSCLGFSPCLVLDDETAAVSVVSRADPKKKRKV |
| 31 | Nucleotide sequence of Casδ-1 system expression cassette |
| | ATGGGACCAAAGAAGAAGAGAAAGGTTATGGATTACAAGGACCACGACGGAGA |
| | CTATAAAGATCATGACATCGATTATAAGGATGATGATGACAAGATGTCTAAAGA |
| | CGGGGACAAAATGAAACACTCCAAGCAAGAAGTTTCCGAGGACAAAGGCGTCA |
| | AGAAAGATCCGTCCGTGTTTCGATGCCAGAACCTCGCCATACTCGAGATGAGGG |
| | AGGAGGTCGCTGACTATTACGCAGACCTTCAGGCTGACTACCGGCACTACCTGC |
| | CTCTGATGATTCTGTGGGGGCAAAATGGCTTTATGGAGCCAGATGAAACGGGCC |
| | TCAACTTCAGCGCCGTCCTGCCGTCGACCGCGAAAACACGCTGTGAGAATAGGT |
| | ATGGTGCCCAGATTGCAGCCGACCTGAAACTCATCGCAGATGATCTGCCGTCTA |
| | GCCTGTATGCTATATTCACGAATGATTACCAGCCTTTAACTGGCGTGAAGAAGGT |
| | TGGTGACCGTTGGAAGTTTGTGACTTCATTGAACACCGTGTCGGATGAGGAATTT |
| | GCAAAGAAAGTCAAACCCCTCCTTCGCGACTACAAAGGTGTCACAGAGGAGCAG |
| | TGGCGTCGTTTCCGTGAGGAACGAGTTGCTGGGAAAACAGCCGACCAGGTTTGG |
| | GACGATCTCGATTCCATCTTGAGGAACCCGACGCTTGCCGCCCTCAAAAAGATT |
| | CATGACAAGCACATGTTCAGAGTTAAGAGGCCGGAGTCAAAGCTGTATTGTGAT |
| | GCATTGGCGCATATGCTCTCATCTTCATATGATTCATGGAAAAAGATCAATGAA |
| | GCACGTCAGTTGGAACTACAGGCCATGAGAGACGAACAACAAGGCCTGCTGGC |
| | AAATAAAACTACTAACTCTCTTTACAATCTGGTCTGTGAGTTCGCGGATGAGCTT |
| | GAATCACAAAACTACGGCCTGACCCGAAGGATATTCTTCATCGCCAGGAAAGAG |
| | TGGGAGGATGAAAAGTCGCACTACCTTCCTGTGATCAAACTCTTGGTGACCGAG |
| | AAGTATCTTCCACTTCTGCAGGCGGATAACAAAACAGTTTCTCCGGCGGTTAAG |
| | GCGTGGGATATTTGCAGCACGTTGAAGCAGCGCAGTCCATATATACGGTTCGTT |
| | CCTCCCACAAAAGACTATCCGATTCCATTTGGCCAGACAGGTAGGGGACACAAG |
| | TTTACACTCAAGGAGGAGGCCGGGAAGGTTTGCTTCCTGGTTCCAGGACTCACC |
| | AAGGATGAAATGCCACTCTCGGGCTCCCACTATTTCTCTAGTCTGCGAATTGACA |
| | AGGTCGGCACAGACTTCAAGATCAGCTTCCGCCATAAGACGAAGATTCGAAGCA |
| | AGAAGTCAGCTAGAGTGACCGTAAAACAACCTCGGATCAACGGCACCGTTACGG |
| | AGGTGGGTATCCTGCGTCGCAACGGCAAGTTTTTCTTACGGGTTGCGTACAAGAT |
| | TGCTTACCCGCAACACAACATTGATCTTAAGAGCTACTTCGCCAGCGCCGCCCCT |
| | TCCACTCAGAAGCTCGAGCTGCTCCCTGCTCAGATGAGGGTGGCGGGAGTCGAT |
| | TTGAACATTTCCGATCCCATCGTCGTATCCAAAGCTGATATTTTTAAGGGAGAGG |
| | ATGGAGGGCCATTATCGGTCTTGGACTACGGATCTGGCAAGGTAGTGGGCGAGC |
| | CCGTCATCATCTGTAAGGATACAAGTCGCGCAGGTAAAATCTCCTCGCTCGCGA |
| | CTAAGTGCCGCACCTTAAGAGACGTCATTCGGAATTTCCGCCACTCTCTCCATCA |
| | GGGAACCCCCCTACCCAGTAAGAGCCTGGAGTTTCTGCAGGAGGTGCCAGTGGC |
| | CCAGGAGGACCTAAAATCGCCATCCCCCAGGTATCTCATACAGACCTGGATAAA |
| | GTATATCAGAACAGAGATGAAGCGCTTGCATTATAAAGTGTGGTTGGACGGCTA |
| | CTGCCACACATCTGAAGCTATGCGTATGCTTGATCTAGTTGATCAGGTCGCCTTC |
| | CTCATGAAGTCATACGAAAACATACATGTGAAGCCGGGCCATAAAATTCCCAAG |
| | AAAACTACCGCGAGTCTCAAGATTAGGGAAACATCCAGGACGAGATTTAGACTG |
| | CATGTATCCAGGTTGGTCGGGGCCAGGGTGGTCCGCGCCTGTGAAGATTGTCAG |
| | ATGATCTTCCTGGAAGACTTGTCGACCGGCTTCTCAGAAGGTAGCAACAATAGC |
| | CTCGCGCGGCTGTTTAGCGCCGGCCAGCTGAAGAATAGTATAGTGATGGCGGCG |
| | GAGAAGGTGGGTAAAGCTGTTGTATTTGTCTGCAAGGATGGAACTAGCATAAAG |
| | GACCCTGTCTTCGGTTTACACGGCCTTCGGCCGTCGAAAAAGACCCCAGGCATC |
| | CCTCCGGATAAAAAAAGACTTTATGTGAGACGGGATGGTAAGATTGGGTACATC |
| | AATGCTGATCAAGCTGCAAGTCTGAATGTACTATTGGTAGGTCTGAGCCATAGT |
| | GTGGCCAAGTACAAGTTCTTCGTCAAAGATGGTAAGTTGCAGGATGACGAAGAG |
| | GTGGCGGCACAACAAGGAAAGAAGCCTAGGAAGATTGGCGTTCGCCTGGAAAG |
| | ACTACTAAAGATGAATTTTGATACCCCGGCCCGCCTTTTTTTTAGCCTGGAGAAG |
| | GACCAGGTGGTTCCGTGCTACGAGGCGACTGAGACGCCTTACACGGGTTGGGTG |
| | TACGTCTACCATGACAGGCTTCTCACAACACGCCAGAGGGACCAACAGGTGAAT |
| | CAACTTGCCCAAGAGGTGAAAGACTTACTTAGGGACGGGGTGAAGATCCCTAAA |
| | TGGGACGTGATCCCAGACTGCGACAACAGTTGCCTCGGGTTCTCTCCCTGCCTGG |
| | TGCTTGACGACGAGACTGCTGCTGTCTCAGTTGTTGGATCTGGGAGCAAGCGGC |
| | CCGCAGCAACGAAAAAGGCAGGGCAAGCAAAAAAGAAGAAATGA |
| 32 | Nucleotide sequence of Casδ-2 system expression cassette |
| | ATGGGTCCGAAAAAGAAGCGGAAAGTTATGGATTATAAGGATCATGATGGCGA |
| | CTATAAGGATCATGACATAGATTATAAAGATGATGACGACAAAATGGAATTGAA |
| | GACAACTTTTTGTCAGTCACTAAAAATTAAGATCCTCCCCGAAAGCCATAAGTTG |
| | TATTACGATGAGCTGCAAGCCGACCTAAAGAAGATGAGAATCTTCTTCGCAACG |
| | CACTCTGGGCTCAACTACAGCTACAAGGAAGATGAAAACGATTCGGAATGGAAG |
| | ATAGCGTCGTCAAAAAAGAGCACACTCTCACCTGAAGCCCAAAGGCTTATTGAC |
| | ATCATCGCCAAAGGCGGTATAGGAGCAGAGCACGCCTACGGAATATTTTACAAG |
| | GACAAAAAGGTGTCGGACAAGATCCGGCGAGATAATTCAAACTGCATCTTCGCC |
| | ATGCTATTACCTAACCTCGTCAATTTCGAGGAAGAATACAAAAAGAGGAAAAGT |
| | AAGCTCCAGCTCACCAAGGAAGAGTGGGAGGAGTACCATAAACGATGTCTTGCA |
| | GGCGAGAGCGTGGAAGTCCTGTGGTCCGAGGAGATTGCTCCAAAAATTGGGCCT |
| | AACGAAGCGGCGAGACTGGCATCACTCGCGTATCACCAGTTCCCAGATTCTCGT |
| | CATCCGGCGGACATTACCCACTGCAGGGCGCTCATGTCTATGGTAGCAGACTCTT |
| | TTTGTTCCTGGGTCGAGTGCCACAAGCTGCACCGTGAGGAAACAGAGAAACTCA |
| | AGACGAAATTACAGGAGAAGATAGAGAGCTGCAAGGAGGCATACAATCTGCTA |
| | CTCGCTTATTCCGAAGACCTGTACAGTAGGAATTATGGTCTGAGTGCCAGGGTTT |
| | TGAAGGCACTTATTGACAACAAATCCCGGTGCTGCTTCGACATAGCCTTTCAGAT |
| | CGCGGAGAAATATCCTGCACTGATGAAGATTGATGAAAAGACCCTGTTCAAGGC |
| | CTACAACGCCCTAAAGCTCCACTGGAAAATAAAACGACGCAAGCCGTTCATCTC |
| | GCTGCCCAAGATCGAGAGGGACTACCAGGTGCCCTTTGGACTCACAGGAAGCAG |
| | AGGCAAGAAGTTCGACGTCTATGTGGAAAACAAGAATATTGTGGTCGAGATCGA |
| | CGGCCAGAAAGTCGAAACCTTCAGCTCTCACTATTTCTCTGATATGCAGATCTCC |
| | AAGATTTACGGTGAAAAGAAAAATTTGAAGGGATTCAAGTTGAAATTCCGCCAC |
| | AAGCTGAAGGACAAGAAGAAGGAGGTTTATGGAGAGTGGATTGAGGCGGAGCT |
| | GAAGGAAATCAAGATTAAGAAAGACATGGAGACAGGCGACTTCTACCTGTATCT |
| | CCCATACACTACAACGCATAATGAGAAAAACTTATTGCTCGAGAAGTTCTTTTCC |
| | TACGCCGATCCACTTAAGGAGACTATTTTTAAGAACGGAAAGACGGAGCTTCCA |
| | AATGAATTTCTAGGCTTCGGCTTCGATTTGAACCTTTCCGACCCGATTGCGATGG |
| | CGGTTGCCGAGTTTGTTCGGGACTCATCAGATGGTGAGATTGGCGCCCTCGACTA |
| | TGGTCACGGTAAGCTCCTCGACGCTAGTGTTTTGGTCTGCAACTCATCACTATCA |
| | AAGCGCATCAATGACCTGGTTGGAGACAATAGAAAGCTGATCCAAGCTATTAGA |
| | TCGTACAAGAACTCGCTAGTAACTAAGGAAATGGACGAGGAGAGTGGCGAGTG |
| | GCTTAAAAAAGCTCTAGGCAATGCAAAGTACGGCAACCATAGGCATCAGCTTCA |
| | ACTGGTCATGTCTAAGCTGAACAAAAAAAGCAAAAAGTACTGGCAGGAATGTCG |
| | CAAGAACGGCCACAACGATCTTTCCGAAAATATTGCACTATTAAAGTTGTTGGA |
| | CGTGCAGTTCTCTTTGCATAAAAGCTATAACAACATACACATCTTTGATTACAAA |
| | AAACAAATTCACAGCCATAAGACTGACTCCAAAAGGGAGAATTTCCGTGAATTT |
| | GTGACGAAGCAATTTGCGGCTACCATAGTAAAGCATTGCAAGGAGGTGATGGCT |
| | AAATATGGCAGAGAGTACGCCGTTGTCTTTTTGGAGGACCTGGAGATGTACTTT |
| | GATGCTGATGCTGATAACAATTCTCTGATCCGGCTTTTTGCACCCGGCCAATTGA |
| | AGAAGTACATCGCCTCCGCTCTGGCTAAGAGCAAGATAGGTTATGTGTTCATCC |
| | CTCCTGCTGGGACAAGTAAGACCGACCCGTTAACTTCGAAAATTGGATTCAGGG |
| | CTACCGGAAAATACTACAAGCAGCTGGGCTATCTGCTTAATAAGTCCGACCTCT |
| | ACGTGGAGCGCAACGGGGTTATCGGGAAGATCAACAGTGATGTAGCTGCGGCG |
| | ATAAATATTCTCTTAAAGGGCGTGAACCACTCAATCGTGCCGTACCGCTTCCTGA |
| | ATAAACATGCTGGGTCTGAACAGAAGCGCCTCAAACGTTTTGAAGGGGAGATCG |
| | GTTGTGATCTGAAAAAGATGCCAAAGACACGCCTCTACTATCTTGACGGAAAAA |
| | TCATCACTGAAGAGGAGAAGAACAGCCTGGAGGAAGCGCTACTCGCTGAGATC |
| | AAGCACTTCCTTCTGTCGAAGGCCAACATTCCGGAGTTTGATGCAATACCGGGG |
| | AGCAATGGCACCTGCAAAGCCTTCTCCGTTCCACGCTGCCTTAAGGGCAGCGGG |
| | AGCAAGCGGCCCGCCGCGACCAAAAAAGCGGGGCAAGCCAAGAAGAAAAAGTG |
| | A |
| 33 | Nucleotide sequence of Casδ-1D31 system expression cassette |
| | ATGGGACCAAAGAAGAAGAGAAAGGTTATGGATTACAAGGACCACGACGGAGA |
| | CTATAAAGATCATGACATCGATTATAAGGATGATGATGACAAGAACCTCGCCAT |
| | ACTCGAGATGAGGGAGGAGGTCGCTGACTATTACGCAGACCTTCAGGCTGACTA |
| | CCGGCACTACCTGCCTCTGATGATTCTGTGGGGGCAAAATGGCTTTATGGAGCC |
| | AGATGAAACGGGCCTCAACTTCAGCGCCGTCCTGCCGTCGACCGCGAAAACACG |
| | CTGTGAGAATAGGTATGGTGCCCAGATTGCAGCCGACCTGAAACTCATCGCAGA |
| | TGATCTGCCGTCTAGCCTGTATGCTATATTCACGAATGATTACCAGCCTTTAACT |
| | GGCGTGAAGAAGGTTGGTGACCGTTGGAAGTTTGTGACTTCATTGAACACCGTG |
| | TCGGATGAGGAATTTGCAAAGAAAGTCAAACCCCTCCTTCGCGACTACAAAGGT |
| | GTCACAGAGGAGCAGTGGCGTCGTTTCCGTGAGGAACGAGTTGCTGGGAAAACA |
| | GCCGACCAGGTTTGGGACGATCTCGATTCCATCTTGAGGAACCCGACGCTTGCC |
| | GCCCTCAAAAAGATTCATGACAAGCACATGTTCAGAGTTAAGAGGCCGGAGTCA |
| | AAGCTGTATTGTGATGCATTGGCGCATATGCTCTCATCTTCATATGATTCATGGA |
| | AAAAGATCAATGAAGCACGTCAGTTGGAACTACAGGCCATGAGAGACGAACAA |
| | CAAGGCCTGCTGGCAAATAAAACTACTAACTCTCTTTACAATCTGGTCTGTGAGT |
| | TCGCGGATGAGCTTGAATCACAAAACTACGGCCTGACCCGAAGGATATTCTTCA |
| | TCGCCAGGAAAGAGTGGGAGGATGAAAAGTCGCACTACCTTCCTGTGATCAAAC |
| | TCTTGGTGACCGAGAAGTATCTTCCACTTCTGCAGGCGGATAACAAAACAGTTTC |
| | TCCGGCGGTTAAGGCGTGGGATATTTGCAGCACGTTGAAGCAGCGCAGTCCATA |
| | TATACGGTTCGTTCCTCCCACAAAAGACTATCCGATTCCATTTGGCCAGACAGGT |
| | AGGGGACACAAGTTTACACTCAAGGAGGAGGCCGGGAAGGTTTGCTTCCTGGTT |
| | CCAGGACTCACCAAGGATGAAATGCCACTCTCGGGCTCCCACTATTTCTCTAGTC |
| | TGCGAATTGACAAGGTCGGCACAGACTTCAAGATCAGCTTCCGCCATAAGACGA |
| | AGATTCGAAGCAAGAAGTCAGCTAGAGTGACCGTAAAACAACCTCGGATCAAC |
| | GGCACCGTTACGGAGGTGGGTATCCTGCGTCGCAACGGCAAGTTTTTCTTACGG |
| | GTTGCGTACAAGATTGCTTACCCGCAACACAACATTGATCTTAAGAGCTACTTCG |
| | CCAGCGCCGCCCCTTCCACTCAGAAGCTCGAGCTGCTCCCTGCTCAGATGAGGG |
| | TGGCGGGAGTCGATTTGAACATTTCCGATCCCATCGTCGTATCCAAAGCTGATAT |
| | TTTTAAGGGAGAGGATGGAGGGCCATTATCGGTCTTGGACTACGGATCTGGCAA |
| | GGTAGTGGGCGAGCCCGTCATCATCTGTAAGGATACAAGTCGCGCAGGTAAAAT |
| | CTCCTCGCTCGCGACTAAGTGCCGCACCTTAAGAGACGTCATTCGGAATTTCCGC |
| | CACTCTCTCCATCAGGGAACCCCCCTACCCAGTAAGAGCCTGGAGTTTCTGCAG |
| | GAGGTGCCAGTGGCCCAGGAGGACCTAAAATCGCCATCCCCCAGGTATCTCATA |
| | CAGACCTGGATAAAGTATATCAGAACAGAGATGAAGCGCTTGCATTATAAAGTG |
| | TGGTTGGACGGCTACTGCCACACATCTGAAGCTATGCGTATGCTTGATCTAGTTG |
| | ATCAGGTCGCCTTCCTCATGAAGTCATACGAAAACATACATGTGAAGCCGGGCC |
| | ATAAAATTCCCAAGAAAACTACCGCGAGTCTCAAGATTAGGGAAACATCCAGGA |
| | CGAGATTTAGACTGCATGTATCCAGGTTGGTCGGGGCCAGGGTGGTCCGCGCCT |
| | GTGAAGATTGTCAGATGATCTTCCTGGAAGACTTGTCGACCGGCTTCTCAGAAG |
| | GTAGCAACAATAGCCTCGCGCGGCTGTTTAGCGCCGGCCAGCTGAAGAATAGTA |
| | TAGTGATGGCGGCGGAGAAGGTGGGTAAAGCTGTTGTATTTGTCTGCAAGGATG |
| | GAACTAGCATAAAGGACCCTGTCTTCGGTTTACACGGCCTTCGGCCGTCGAAAA |
| | AGACCCCAGGCATCCCTCCGGATAAAAAAAGACTTTATGTGAGACGGGATGGTA |
| | AGATTGGGTACATCAATGCTGATCAAGCTGCAAGTCTGAATGTACTATTGGTAG |
| | GTCTGAGCCATAGTGTGGCCAAGTACAAGTTCTTCGTCAAAGATGGTAAGTTGC |
| | AGGATGACGAAGAGGTGGCGGCACAACAAGGAAAGAAGCCTAGGAAGATTGGC |
| | GTTCGCCTGGAAAGACTACTAAAGATGAATTTTGATACCCCGGCCCGCCTTTTTT |
| | TTAGCCTGGAGAAGGACCAGGTGGTTCCGTGCTACGAGGCGACTGAGACGCCTT |
| | ACACGGGTTGGGTGTACGTCTACCATGACAGGCTTCTCACAACACGCCAGAGGG |
| | ACCAACAGGTGAATCAACTTGCCCAAGAGGTGAAAGACTTACTTAGGGACGGGG |
| | TGAAGATCCCTAAATGGGACGTGATCCCAGACTGCGACAACAGTTGCCTCGGGT |
| | TCTCTCCCTGCCTGGTGCTTGACGACGAGACTGCTGCTGTCTCAGTTGTTGGATC |
| | TGGGAGCAAGCGGCCCGCAGCAACGAAAAAGGCAGGGCAAGCAAAAAAGAAG |
| | AAATGA |
| 34 | PAM library sequence |
| | NNNNNNNNGGTATAACAACTTCGACGAGCTCTACA |
| 35 | Target sequences for PAM recognition site identification of Casδ-1 |
| | GGUAUAACAACUUCGACGAGCUCUACA |
| 36 | Guide sequence of in vitro enzyme digestion of Casδ-1 |
| | GGUAUAACAACUUCGACGAGCUCUACA |
| 37 | Guide sequence of Casδ-1 in maize |
| | CGGUGGGCUGGCGCUGGGGUUCAGCU |
| 38 | Guide sequence of sg1 of Casδ-1 in human cells |
| | GAGCCAGAGAGGAUCCUGGGAGGGAG |
| 39 | Guide sequence of sg2 of Casδ-1 in human cells |
| | UGACUUUGUCACAGCCCAAGAUAGUU |
| 40 | Guide sequence of sg3 of Casδ-1 in human cells |
| | AAACCCAGACACAUAGCAAUUCAGGA |
| 41 | Guide sequence of sg4 of Casδ-1 in human cells |
| | CUGAGGGGCUGCUGGUUUGGCUGGUG |
| 42 | Guide sequence of sg5 of Casδ-1 in human cells |
| | GAGAUGCCAGCAGAAGUUGGGCAGAA |
| 43 | Guide sequence of sg6 of Casδ-1 in human cells |
| | GGGCAGAGUGGAGAUGGUGGGGACAA |
| 44 | Guide sequence of sg7 of Casδ-1 in human cells |
| | ACUAGGGUGGGCAACCACAAACCCAC |
| 45 | Guide sequence of sg8 of Casδ-1 in human cells |
| | UGUACAGAAGGCUGAAAGGAGAGAAC |
| 46 | Guide sequence of sg9 of Casδ-1 in human cells |
| | CGGUACCAGUUUAGCACGAAGCUCUC |
| 47 | Guide sequence of sg10 of Casδ-1 in human cells |
| | CCACCAUUGUCUUUCCUAGCGGAAUG |
| 48 | Guide sequence of sg11 of Casδ-1 in human cells |
| | AAGCACUGUGGGUACGAAGGAAAUGA |
| 49 | Guide sequence of sg12 of Casδ-1 in human cells |
| | UGUCACAAAGUAAGGAUUCUGAUGUG |
| 50 | Guide sequence of sg13 of Casδ-1 in human cells |
| | CUGUUGUUGAAGGCGUUUGCACAUGC |
| 51 | Guide sequence of sg14 of Casδ-1 in human cells |
| | UCUGCAGGCCAGAUGAGGGCUCCAGA |
| 52 | Guide sequence of AAVS1-ATG PAM in Casδ-1 human cells |
| | GAGCCAGAGAGGAUCCUGGGAGGGAG |
| 53 | Guide sequence of AAVS1-ATA PAM in Casδ-1 human cells |
| | GACCACUGUGUGGGGGUAAAGGACCU |
| 54 | Guide sequence of AAVS1-ACA PAM in Casδ-1 human cells |
| | CCCCCAUUUCCUGGAGCCAUCUCUCU |
| 55 | Guide sequence of AAVS1-GCA PAM in Casδ-1 human cells |
| | AACCUUAGAGGUUCUGGCAAGGAGAG |
| 56 | Guide sequence of AAVS1-GTA PAM in Casδ-1 human cells |
| | AGCAAACCUUAGAGGUUCUGGCAAGG |
| 57 | Guide sequence of AAVS1-ACG PAM in Casδ-1 human cells |
| | AUGGAGCCAGAGAGGAUCCUGGGAGG |
| 58 | Guide sequence of AAVS1-GTG PAM in Casδ-1 human cells |
| | GGAGGGAAGGGGGGGAUGCGUGACCU |
| 59 | Guide sequence of AAVS1-GCG PAM in Casδ-1 human cells |
| | UGACCUGCCCGGUUCUCAGUGGCCAC |
| 60 | Guide sequence of Casδ-1T26 |
| | GAGCCAGAGAGGAUCCUGGGAGGGAG |
| 61 | Guide sequence of Casδ-1T24 |
| | GAGCCAGAGAGGAUCCUGGGAGGG |
| 62 | Guide sequence of Casδ-1T22 |
| | GAGCCAGAGAGGAUCCUGGGAG |
| 63 | Guide sequence of Casδ-1T21 |
| | GAGCCAGAGAGGAUCCUGGGA |
| 64 | Guide sequence of Casδ-1T20 |
| | GAGCCAGAGAGGAUCCUGGG |
| 65 | Guide sequence of Casδ-1T19 |
| | GAGCCAGAGAGGAUCCUGG |
| 66 | Guide sequence of Casδ-1T18 |
| | GAGCCAGAGAGGAUCCUG |
| 67 | Guide sequence of Casδ-1T16 |
| | GAGCCAGAGAGGAUCC |

### Specific Models for Carrying Out the Invention

The present invention is now described with reference to the following examples which are intended to illustrate the present invention (but not to limit the present invention).

Unless otherwise specified, the experiments and procedures described in the examples were basically performed according to the methods known in the art and using conventional methods described in various references. For example, conventional techniques of immunology, biochemistry, chemistry, molecular biology, microbiology, cell biology, genomics and recombinant DNA used in the present invention can be found in Sambrook, Fritsch, and Maniatis, MOLECULAR CLONING: A LABORATORY MANUAL, 2nd ed. (1989); CURRENT PROTOCOLS IN MOLECULAR BIOLOGY, edited by F. M. Ausubel et al. (1987)); METHODS IN ENZYMOLOGY series, Academic Press: PCR 2: A PRACTICAL METHOD APPROACH, edited by M. J. MacPherson, B. D. Hames, and G. R. Taylor (1995); ANTIBODIES, A LABORATORY MANUAL, edited by Harlow and Lane (1988); and, ANIMAL CELL CULTURE, edited by R. I. Freshney (1987).

In addition, when specific conditions were not specified in the examples, they were carried out under conventional conditions or conditions recommended by the manufacturer. The reagents or instruments used without indicating the manufacturer were all conventional products that could be obtained commercially. It is known to those skilled in the art that the examples describe the present invention by way of example, and are not intended to limit the scope sought to be protected by the present invention. All publications and other references mentioned herein are incorporated herein by reference in their entirety.

The sources of some reagents involved in the following examples were as follows:

LB liquid culture medium: 10 g of tryptone, 5 g of yeast extract, 10 g of NaCl, diluted to 1 L, and sterilized. If the addition of an antibiotic was required, it was added after the culture medium was cooled down, and its final concentration was 50 μg/mL.

Chloroform/isoamyl alcohol: 240 mL of chloroform was added with 10 mL of isoamyl alcohol, and mixed well.

RNP buffer: 100 mM sodium chloride, 50 mM Tris-HCl, 10 mM MgCl₂, 100 μg/mL BSA, pH 7.9.

Prokaryotic expression vectors pET-30a, pUC19, and pACYCDuet-1 were purchased from Beijing Quanshijin Biotechnology Co., Ltd.

Escherichia coli competent TSC-E03 was purchased from Beijing Qingke Biotechnology Co., Ltd.

### Example 1. Acquisition of Casδ Sequences and Casδ Guide RNA

1. Annotation of CRISPR and genes: Prodigal was used to perform the gene annotation of the data of microbial genome and metagenome of NCBI and JGI databases to obtain all proteins, and Piler-CR was used to perform the annotation of CRISPR loci, and the parameters were all default parameters.

2. Protein filtering: The annotated proteins were subjected to redundancy removal through sequence consistency so as to remove proteins with completely identical sequences.

3. Acquisition of CRISPR-related proteins: Each CRISPR locus was extended by 10 Kb upstream and downstream, and the non-redundant proteins in the CRISPR adjacent interval were identified.

4. Clustering of CRISPR-related proteins: BLASTP was used to perform internal pairwise alignment of non-redundant CRISPR-related proteins, and the alignment results with Evalue<1E−10 were outputted. MCL was used to perform clustering analysis on the output results of BLASTP, CRISPR-related protein families.

5. Identification of CRISPR-enriched protein families: BLASTP was used to align the proteins of the CRISPR-related protein families to the non-redundant proteins databases from which the non-CRISPR-related proteins were removed, and the alignment results with Evalue<1E−10 were outputted. If the homologous proteins found in a non-CRISPR-related protein database were less than 100%, it meant that the proteins of this family were enriched in the CRISPR region. In this way, the CRISPR-enriched protein families were identified.

6. Annotation of protein functions and domains: The CRISPR-enriched protein family was annotated using the Pfam database, the NR database, and the Cas proteins collected from NCBI to obtain a new CRISPR/Cas protein family. Multiple sequence alignment of each CRISPR/Cas family protein was performed using Mafft, and then conserved domain analysis was performed using JPred and HHpred to identify the protein family containing RuvC domain.

On this basis, the inventors obtained some new Cas effector proteins, which were named Casδ-1 and Casδ-2, respectively, the sequences of the proteins were set forth in SEQ ID NO: 1 and SEQ ID NO: 2, and the nucleotide sequences encoding the proteins were as set forth in SEQ ID NO: 4 and SEQ ID NO: 5. The direct repeat sequences (the repeat sequences contained in pre-crRNA) corresponding to Casδ-1 and Casδ-2 were set forth in SEQ ID NO: 7 and SEQ ID NO: 8.

### Example 2. Description of Sequence Structure of Casδ Gene

1. The CRISPR/Casδ sequence fragment was synthesized by Beijing Qingke Biotechnology Co., Ltd. and constructed into the protein expression vector pET-30a (+), and the first generation sequencing was performed for confirmation. According to the sequencing results, the recombinant plasmid pET-30a+CRISPR/Casδ was described as follows:

(1) The recombinant plasmid pET-30a+CRISPR/Casδ-1 contained an expression cassette, and the expression cassette sequence was set forth in SEQ ID NO: 31. In the sequence as set forth in SEQ ID NO: 31, from the 5′ end, positions 1 to 27 were the nucleotide sequence of SV40-NLS, positions 28 to 96 were the nucleotide sequence of 3×FLAG, positions 97 to 2904 are were nucleotide sequence of Casδ-1, and positions 2905 to 2964 were the nucleoplasmin NLS signal peptide.

(2) The recombinant plasmid pET-30a+CRISPR/Casδ-2 contained an expression cassette, and the expression cassette sequence was set forth in SEQ ID NO: 32. In the sequence as set forth in SEQ ID NO: 32, from the 5′ end, positions 1 to 27 were the nucleotide sequence of SV40-NLS, positions 28 to 96 were the nucleotide sequence of 3×FLAG, positions 97 to 2697 were the nucleotide sequence of Casδ-2, and positions 2698 to 2757 were the nucleoplasmin NLS signal peptide.

### Example 3. Identification of PAM and DNA Cleavage Mode of CRISPR/Casδ System

### I. In Vitro Expression and Purification of Casδ Protein

The specific steps of in vitro expression and purification of Casδ protein were as follows:

1. Artificial synthesis of nucleotide sequences as set forth in SEQ ID NOs: 31 to 32.

2. The recombinant plasmids pET-30a-CRISPR/Casδ-1 and pET-30a-CRISPR/Casδ-2 were introduced into E. coli TSC-E03 to obtain recombinant bacteria, and the recombinant bacteria were named TSC-E03-CRISPR/Casδ-1 and TSC-E03-CRISPR/Casδ-2. The single clones of TSC-E03-CRISPR/Casδ-1 and TSC-E03-CRISPR/Casδ-2 were picked out, inoculated into 100 mL of LB liquid culture medium (containing 50 μg/mL kanamycin), and cultured under shaking at 37° C. and 200 rpm for 12 h to obtain culture solutions.

3. The culture solutions were taken and inoculated into 50 mL of LB liquid culture medium (containing 50 μg/mL kanamycin) at a volume ratio of 1:100, cultured under shaking at 37° C. and 200 rpm until the OD_{600 nm} value was 0.6, then IPTG was added to have a concentration of 1 mM, cultured under shaking at 18° C. and 220 rpm for 14 h, and centrifuged at 4° C. and 7000 rpm for 10 min to obtain bacterial precipitates.

5. The bacterial precipitates were taken, added with 100 mL of pH 8.0, 100 mM Tris-HCl buffer, resuspended and ultrasonically disrupted (ultrasonic power was 600 W, and cycle program was: disruption 4 s, stop 6 s, total 20 min), and then centrifuged at 4° C., 10000 rpm for 10 min to collect Supernatant A.

6. Supernatant A was taken, centrifuged at 4° C., 12000 rpm for 10 min to collect Supernatant B.

7. The nickel column produced by GE was used to purify Supernatant B (referring to the instructions of the nickel column for the specific steps of purification), and then the protein quantification kit produced by Thermo Fisher was used to quantify Casδ-1 to Casδ-3 proteins.

### II. Transcription and Purification of Casδ Protein Guide RNA:

1. The templates for guide RNA transcription were designed respectively. The structure of the transcription templates were: (1) T7 promoter+direct repeat sequence of Casδ-1 and Casδ-2 (SEQ ID NOs: 7 to 8)+guide sequence (SEQ ID NO: 36). The primers were designed using Primer5.0 software to ensure that the Forward primer and Reward primer had at least 18 bp of overlapping sequence.

2. The following reaction system was prepared, gently blown and beaten and mixed well, then centrifuged briefly, and placed in a PCR instrument for slow annealing. The PCR system was as follows:

| Component | Volume (μL) |
|---|---|
| Forward Primer (100 nM) | 7.5 |
| Reward Primer (100 nM) | 7.5 |
| 2*KAPA Mix | 25 |
| ddH₂O | 10 |
| Total volume | 50 |

3. MinElute PCR Purifcation Kit was used to purify the template, and the steps were as follows:
1) The PCR product was added with PB of 5 times volume, and a MinElute column was placed on a 2 mL collection tube, allowed to stand at room temperature for 2 min, and centrifuged at 12000 g for 1 min;
2) The waste liquid was discarded, and 750 μL of Buffer PE (ethanol was added before use) was added and centrifuged at 12000 g for 1 min;
3) The waste liquid was discarded, 350 μL of Buffer PE was added and centrifuged at 12000 g for 1 min, then the waste liquid was added, and centrifugation was performed at 12000 g for 2 min;
4) The MinElute column was placed on a new 1.5 mL centrifuge tube, the lid was opened, and standing was performed at 65° C. for 2 min;
5) 20 μL of preheated EB solution was added, allowed to stand for 2 min, and centrifuged at 12000 g for 2 min. In order to improve the recovery rate, the content of the centrifuge tube could pass through the MinElute centrifuge column 2 to 3 times;
6) The template was measured for concentration by Nanodrop, and frozen at −20° C. for later use.

4. Purification of guide RNA: DNaseI in the system was extracted and removed with phenol: chloroform: isoamyl alcohol (25:24:1);
1) 80 μL of RNA free H₂O was added to the post-transcription reaction system to adjust the volume to 100 L;
2) 2 mL of Phase Lock Gel (PLG) Heavy was taken out, centrifuged at 15000 g for 2 min, and added with 100 μL of phenol: chloroform: isoamyl alcohol (25:24:1), and 100 μL of RNA digested with DNAseI, and the Phase-Lock tube was gently flicked 5 to 10 times by hand to mix evenly, and then centrifuged at 15° C. and 16000 g for 12 min;
3) A new RNA-free 1.5 mL centrifuge tube was taken, the supernatant was pipetted from the previous centrifugation and added to the centrifuge tube without pipetting the gel, then added with isopropanol of the same volume as the supernatant and sodium acetate solution of the one-tenth the volume, mixed well with a pipette tip, and placed into a −20° C. refrigerator for 1 h or overnight;
4) Centrifugation was performed at 4° C., 16000 g for 30 min, the supernatant was discarded, 75% pre-cooled ethanol was added, the precipitate was mixed well by pipetting, and centrifuged at 4° C., 16000 g for 12 min, the supernatant was discarded, then it was allowed to stand in a fume hood for 2 to 3 min, the ethanol on RNA surface was dried in the air, 100 μL of RNA free H₂O was added, and mixed well by pipetting.

5. The purified crRNA was measured for concentration by Nanodrop, and uniformly diluted to 250 ng/μL, divided into 200 μL PCR centrifuge tubes, and frozen at −80° C. for later use.

### III. Casδ Protein In Vitro Enzyme Digestion and PAM Consumption:

### 1. Establishment of Double-Stranded DNA Enzyme Digestion System:

(1) The following reaction system was prepared, gently pipetted and mixed well, and then centrifuged briefly. It was placed at 37° C. for 15 min; and the DNA cleavage reaction system was as follows:

| Component | Sample amount | |
|---|---|---|
| 12δ-crRNA (250 ng/μL) | 600 | ng |
| 12δ protein (0.5 μg/μL) | 0.5 | μg |
| 10*DNA Cleavage buffer | 1 | μL |

| | |
|---|---|
| RNA-Free H₂O | Supplemented to 7 μL |

(2) 300 ng of substrate DNA (100 ng/μL), 3 μL, was added, gently pipetted to mix well and then centrifuged briefly. It was placed at 37° C. for 8 h;
(3) RNAse was added, placed at 37° C. for 15 min to fully digest the RNA impurities in the system;
(4) Proteinase K was added, placed at 58° C. for 15 min to digest Casδ-1 to 3 proteins;
(5) Detection was performed by running agarose gel.

The gel results showed that Casδ-1 was capable of effectively cleaving double-stranded DNA.

### 2. Identification of PAM Site:

(1) The reaction system as in step 6 above was prepared, the substrate DNA was replaced with a plasmid library with 8 random bases before target, and placed at 37° C. for 8 h, and the secondary control sample was a sample with Casδ added but no crRNA added. Three repeats were set for each protein;
(2) After the reaction, the reaction sample was subjected to column purification, and the purified product was used as a template to construct the second-generation library. The system and method for library construction were the same as the library construction method in step 2 of PAM library consumption in Escherichia coli. The specific operation process was as follows:

(Each sample corresponded to one R-directed primer, and corresponded to multiple F-directed primers), the following reagents were prepared:

| Reagent | Usage amount | |
|---|---|---|
| Template | 20 | ng |
| High-fidelity PCR mix | 20 | μL |
| NGS-Lib-Fwd-1-10 | 2 | μL |
| NGS-Lib-Rev | 2 | μL |

| | |
|---|---|
| distilled water | Supplemented to 40 μL |

The prepared reaction system was loaded in a PCR instrument, and the program was as follows:

| Temperature | Time | |
|---|---|---|
| 98° C. | 3 | min |
| 98° C. | 15 | s |
| 60° C. | 30 | s |
| 72° C. | 20 | s |
| Go to step 2 | 20 | cycles |
| 72° C. | 5 | min |

| | |
|---|---|
| 10° C. | forever |

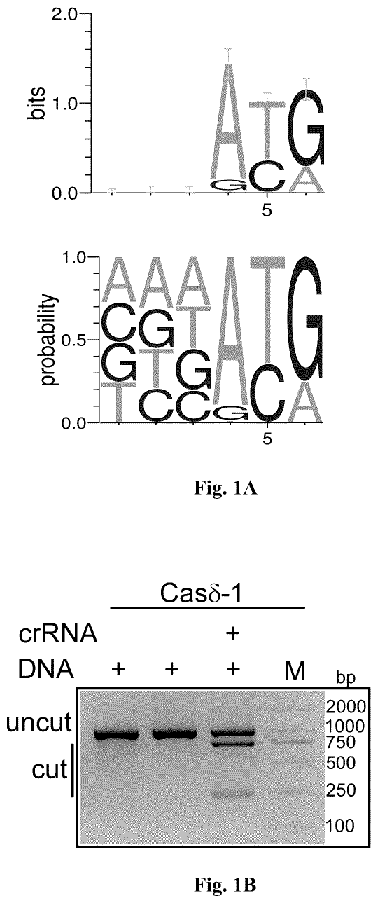

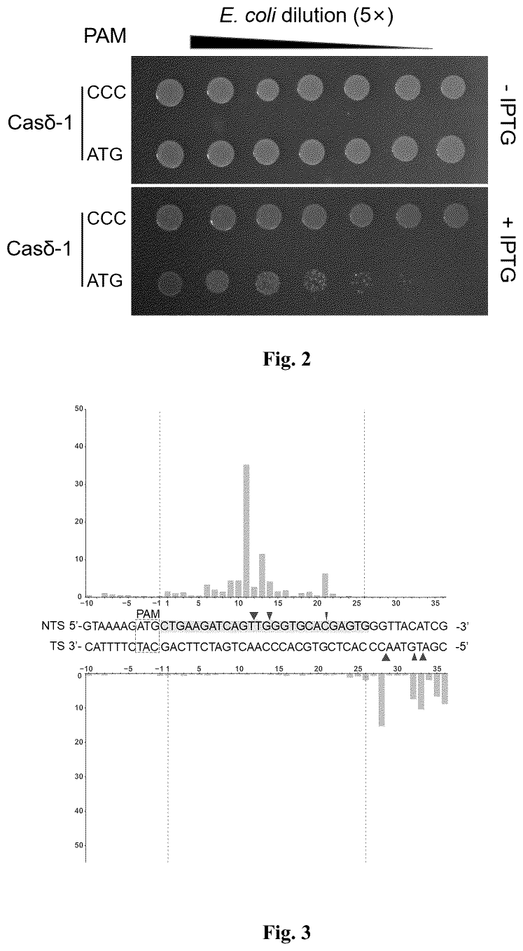

Sequencing 1G for each sample;
(3) The numbers of occurrences of the combined PAM sequences in the experimental group and the control group were counted, respectively, and standardized with the number of all PAM sequences in each group. For any PAM sequence, when log 2 (normalized value of the control group/normalized value of the experimental group) was greater than 3.5, it was believed that this PAM was significantly consumed. The significantly consumed PAM sequences were obtained from all PAM sequences. In addition, Weblogo was used to predict the significantly consumed PAM sequences, and finally the PAM domains of Casδ were obtained (FIG. 1A and FIG. 1B).
(4) Verification of PAM library domains: Through the PAM library consumption experiment, we obtained the PAM domain of Casδ-1. In order to verify the rigor of this domain, we set up ATG PAM for in vivo experiments to test the editing activity of Casδ-1 on this PAM. First, we integrated the 26 nt target of the T7 promoter with the corresponding PAM site and the sequence of the T7 terminator into the vector pET30a-Casδ-1, which was then co-transfected with the pACYCDuet-1 plasmid and coated on kanamycin and chloramphenicol resistance plates for screening. The monoclonal plaques with double resistance were selected for shaking bacteria, and IPTG induction was performed for 12 hours at an OD value of 1.0. Then, the bacteria before and after induction were observed by gradient dilution. If the chloramphenicol gene was edited, the growth on the chloramphenicol resistance plate was poor. Through the experimental results (FIG. 2 and FIG. 3), we could see that CRISPR/Casδ-1 could only effectively edit target sequences with specific PAM domains (e.g., ATG), but had no editing activity on the rest of the target sequences (e.g., CCC), thus verifying the accuracy of Casδ-1 for recognition of PAM domains. Through the above experimental results, it was confirmed that Casδ-1 had a rigorous PAM recognition mode, so Casδ-1 could significantly reduce off-target effects.
(5) Analysis of dsDNA cleavage mode of Casδ-1 protein:

In order to further determine the cleavage site and substrate cleavage mode of Casδ-1 protein when targeting dsDNA, the cut bands after enzymatic digestion of Casδ-1 protein-targeted substrate dsDNA were individually cut and recovered. Since the dsDNA fragments were designed with the 26 nt target site at a greater distance from the 5′ end than from the 3′ end, bands with larger fragment lengths were subjected to the first-generation sequencing using F-direct primers that amplified linear dsDNA fragments, while the recovered bands with shorter fragment lengths were sequenced with R-direct primers as amplified. By aligning the results of the first-generation sequencing to the reference dsDNA sequence, the cleavage site of the Casδ-1 protein for the substrate was located at 18 nt and 19 nt far from the PAM, thereby forming a sticky end with a 1 nt protrusion (FIG. 3).

### Example 4. Analysis of Trans-Cleavage Activity of CRISPR/Casδ System

A single-stranded or double-stranded DNA without SEQ ID NO: 36 was designed as the enzyme substrate for trans-cleavage activity analysis; a single-stranded or double-stranded DNA containing SEQ ID NO: 36 was used as the targeted substrate, and the purified protein and the corresponding crRNA were prepared into the following reaction system as shown in the table below.

| Component | Volume | |
|---|---|---|
| Cas protein | 1 μL | (60 nM) |
| crRNA | 1 μL | (120 nM) |
| ssDNA | 1 μL | (30 nM) |

| | |
|---|---|
| 10X ssDNA Cleavage Buffer | 2 μL |

The above reaction system was placed in a PCR instrument and incubated at 25° C. for 10 min; then 2 μL of single-stranded DNA without target sequence was added and incubated at 37° C. for 1 h; 1 μL of RNase A was added and incubated at 37° C. for 30 min; 1 μL of Proteinase K was added and incubated at 55° C. for 30 min; 4 μL of 6×DNA loading buffer was added, and detection was performed by running 1.5% agarose gel. If the targeted substrate was dsDNA, it was necessary to synthesize a primer of about 90 nt without target sequence, which was diluted to 1 μM, packaged and stored at −20° C. as a random substrate in the trans-cleavage experiment. The dsDNA fragments in the in vitro dsDNA enzyme cleavage experiment were used as the targeted substrate, and the purified proteins and corresponding crRNA were prepared into the reaction system as shown in the following table.

| Component | Volume | |
|---|---|---|
| Cas protein | 1 μL | (200 nM) |
| crRNA | 1 μL | (800 nM) |
| dsDNA | 1 μL | (30 nM) |

| | |
|---|---|
| 10X ssDNA Cleavage Buffer | 2 μL |
| Nuclease-free water | Supplemented to 18 μL |

The above reaction system was placed in a PCR instrument and incubated at 37° C. for 15 min; (1) 2 μL (100 nM) of diluted random substrate ssDNA was added and incubated at 37° C. for 2 h; (2) 1 μL of RNase A was added and incubated at 37° C. for 30 min; (3) 1 μL of Proteinase K was added and incubated at 55° C. for 30 min; (4) 2×RNA Loading Buffer was added, and detection was performed by running 10% nucleic acid denaturing acrylamide gel.

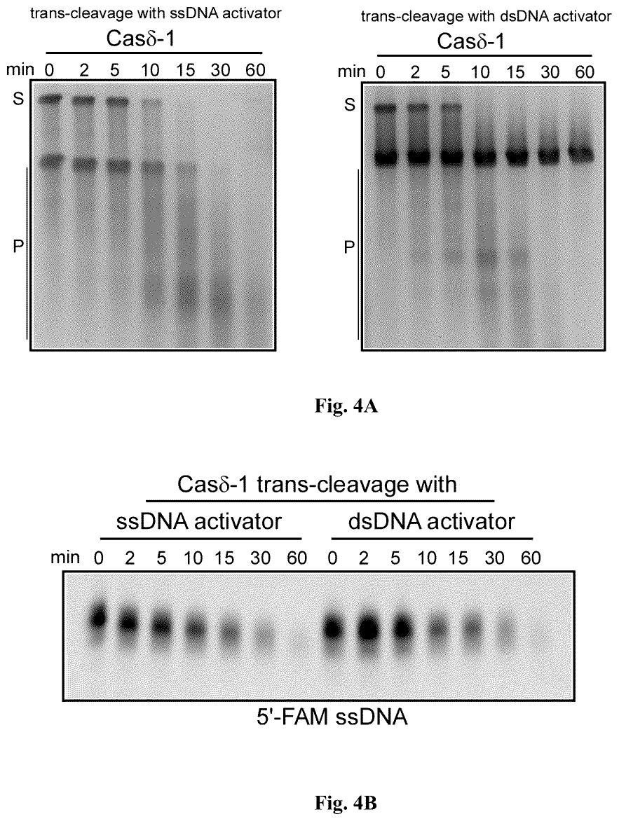

The experimental results were shown in FIG. 4A and FIG. 4B, and the experimental results confirmed that after the trans-cleavage activity of Casδ-1 protein was activated, both single-stranded DNA and double-stranded DNA substrates could be cleaved.

### Example 5. Detection of Editing Activity of Casδ in Human Cells

In this example, 14 target sites, i.e., sg1 to sg14, were selected from the genome sequence of human HELA cells, and 14 guide sequences were designed for these target sites (the sequences were set forth in SEQ ID NO: 38 to SEQ ID NO: 51, respectively). Further, the eukaryotic expression vector containing Casδ-1 gene and the expression vector containing U6 promoter and guide RNA (containing the direct repeat sequence as set forth in SEQ ID NO: 7 and the editing guide sequence in human cells containing SEQ ID NO: 38 to SEQ ID NO: 51) were transferred into human HELA cells by liposome transfection, and cultured at 37° C. and 5% carbon dioxide concentration for 72 hours. DNA from all cells was extracted, and the sequences containing 200 bp of the target site were amplified. The PCR products were sent to Beijing Jiyinjia Medical Testing Laboratory Co., Ltd. for second-generation library construction and sequencing.

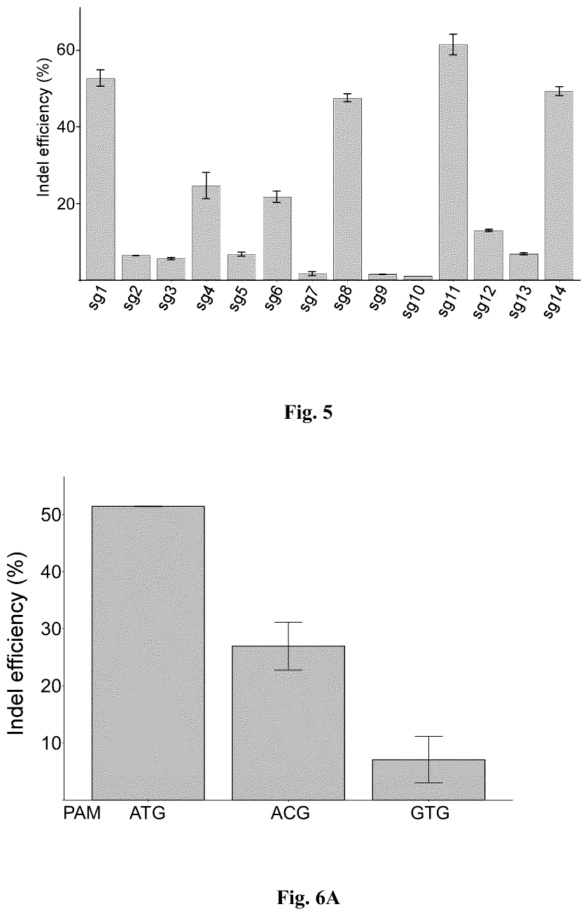

The sequencing results were statistically analyzed by bioinformatics and the original target sequence. The results showed that Casδ-1 could effectively edit all 14 target sites, and the editing efficiency could reach up to about 60% (FIG. 5).

### Example 6. Analysis of Editing Activity of Different PAM Recognition Sites of Casδ-1 in HELA Cells

The PAM site recognized by Casδ-1 was identified as RYR (where R was A or G, and Y was T or C) by the experimental method of Example 3. In order to further confirm whether all recognized PAM sites had editing efficiency in eukaryotes, their editing efficiency was verified in HELA cells. Eight guide sequences were designed for these PAM recognition sites (the sequences were set forth in SEQ ID NO: 52 to SEQ ID NO: 59, respectively). The target sequences of different PAM recognition sites were selected within the 200 bp range of the AAVS1 gene, and the expression vector containing U6 promoter and guide RNA (containing the direct repeat sequence as set forth in SEQ ID NO: 7 and the editing guide sequence in human cells containing SEQ ID NO: 52 to SEQ ID NO: 59) was transferred into human HELA cells by liposome transfection and cultured at 37° C. and 5% carbon dioxide concentration for 72 hours.

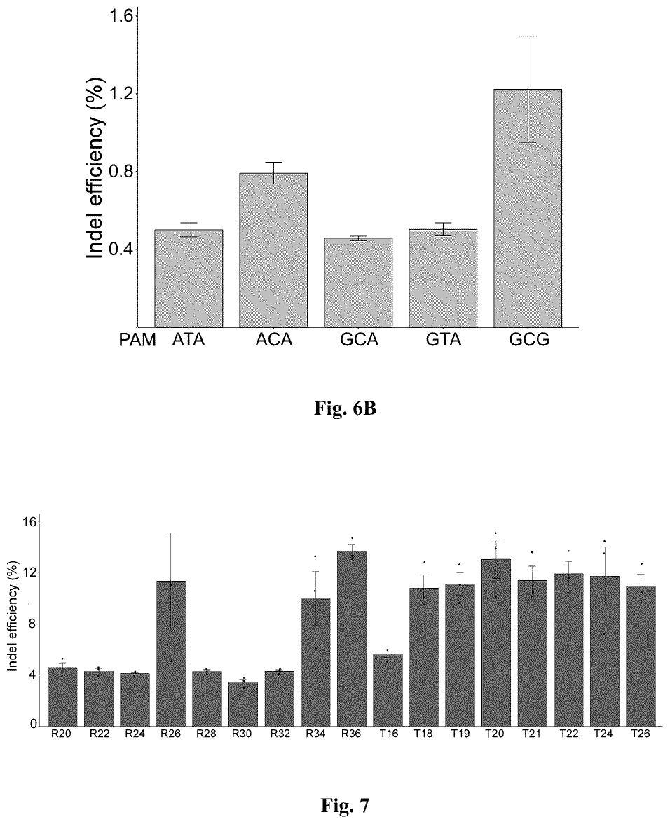

Through the second-generation library construction, second-generation sequencing and bioinformatics analysis of the transformed samples, it was found that the RYR PAM recognition sites of Casδ-1 all had different degrees of editing efficiency, among which the PAM recognition sites of ATG and ACG had higher editing efficiency (FIG. 6A and FIG. 6B).

### Example 7. Analysis of Editing Activity of Casδ-1 with Different Repeat and Target Lengths

In order to further verify the effects of direct repeat sequences (Repeat) and guide sequences (Target) with different lengths on editing efficiency, we selected Repeat (SEQ ID NO: 7 or 9 to 16) and Target (SEQ ID NO: 38) sequences with different lengths for combination, and Target (SEQ ID NO: 60 to 67) and Repeat (SEQ ID NO: 7) sequences with different lengths for combination, and confirmed their differences in editing efficiency in eukaryotes by animal cell transfection. The specific experimental procedures and steps were referred to Example 5.

Through bioinformatics analysis, it was found that Repeat still retained a certain editing activity when 18 bases or less were cut off from the 5′ end, and Target had editing activity in the range of 16 to 26 bases (FIG. 7).

### Example 8. Analysis of Editing Activity of Casδ-1 in Stable Genetic Transformation of Maize

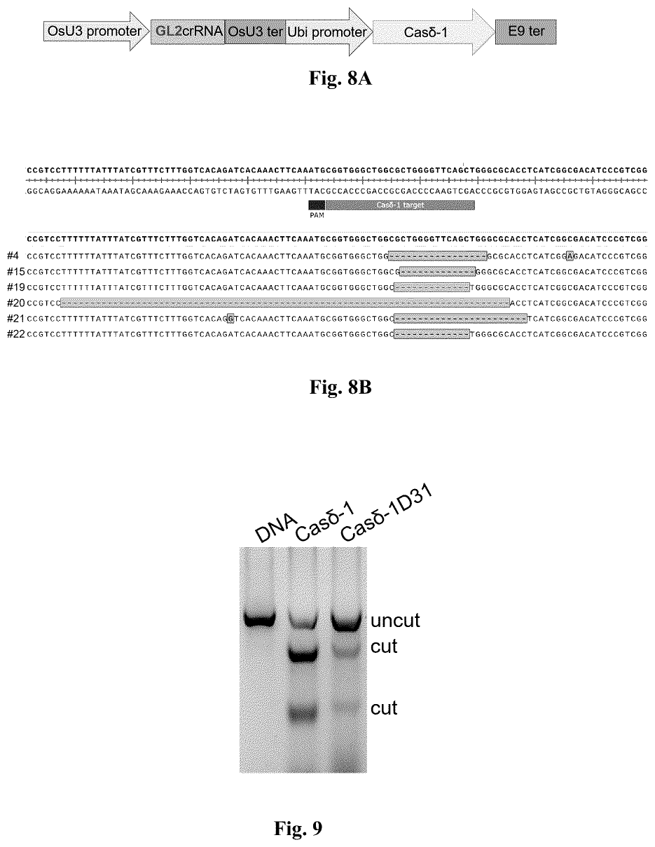

The target site of maize ZmGL2 gene was designed (wherein, the direct repeat sequence of Casδ-1 protein was set forth in SEQ ID NO: 7, and the guide sequence was set forth in SEQ ID NO: 37), and the crRNA sequence was ligated to the expression vector of OsU3 promoter, and the Ubi promoter and Casδ-1 protein coding sequence were constructed into the same expression vector (FIG. 8A). Positive plants were obtained by genetic transformation screening of maize callus, and the first-generation sequencing experimental method was used to analyze whether the ZmGL2 target site was edited, and a total of 6 mutants of different deletion types were obtained (FIG. 8B).

The above experiments confirmed that the Casδ-1 protein successfully edited the target site in maize.

### Example 9. Analysis of Editing Activity of Casδ-1 Truncated Protein

In order to further reduce the protein size and improve the delivery efficiency, we deleted the 31 amino acids at the N-terminal of Casδ-1 to obtain a truncated form of Casδ-1, and named the truncated form Casδ-1D31 (the amino acid sequence was set forth in SEQ ID NO: 3, and the nucleotide sequence was set forth in SEQ ID NO: 6). Furthermore, the amino acid sequence of the Casδ-1D31-NLS fusion protein as set forth in SEQ ID NO: 30 was synthesized based on its amino acid sequence, and constructed into the pET30a vector by homologous recombination experimental method. The steps of prokaryotic protein purification and in vitro enzyme digestion experiment were the same as those described in Example 3.

The results of in vitro enzyme digestion verification showed that Casδ-1D31 still had double-stranded DNA cleavage activity (FIG. 9).

Although the specific models of the present invention have been described in detail, those skilled in the art will understand that various modifications and changes can be made to the details based on all the teachings that have been disclosed, and these changes are within the scope of protection of the present invention. All of the invention is given by the appended claims and any equivalents thereof.

What is claimed:

1. A protein, which comprises or consists of a sequence selected from the following:
(i) a sequence as set forth in any one of SEQ ID NOs: 1, 2, and 3;
(ii) a sequence having a substitution, deletion, or addition of one or more amino acids as compared to the sequence as set forth in any one of SEQ ID NOs: 1, 2, and 3;
(iii) a sequence having a substitution, deletion, or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 amino acids as compared to the sequence as set forth in any one of SEQ ID NOs: 1, 2, and 3; or
(iiii) a sequence having a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in any one of SEQ ID NOs: 1, 2, and 3.

2. A truncated protein, wherein the truncated protein has a truncation of 1 to 10, 11 to 20, 21 to 30, 31 to 40, 41 to 50, 51 to 60 or more amino acids at the N-terminal and/or C-terminal as compared to the protein according to claim 1.

3. The truncated protein according to claim 2, the truncated protein is characterized by one or more of the following:
(1) the truncated protein has a truncation of 31 amino acids at the N-terminal as compared to the sequence as set forth in any one of SEQ ID NO: 1 or 2;
(2) the truncated protein comprises a sequence selected from the following, or consists of a sequence selected from the following:
(i) a sequence as set forth in SEQ ID NO: 3;
(ii) a sequence having a substitution, deletion, or addition of one or more amino acids as compared to the sequence as set forth in SEQ ID NO: 3;
(iii) a sequence having a substitution, deletion, or addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 as compared to the sequence as set forth in SEQ ID NO: 3; or
(iiii) a sequence having a sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% as compared to the sequence as set forth in SEQ ID NO: 3.

4. A conjugate, which comprises the protein according to claim 1 or a truncated protein comprising the protein and a modification portion;
wherein, the modification portion is selected from an additional protein or polypeptide, a detectable label, and any combination thereof.

5. The conjugate according to claim 4, the conjugate is characterized by one or more of the following:
(1) the modification portion is connected to the N-terminal or C-terminal of the protein or truncated protein optionally via a linker;
(2) the modification portion is fused to the N-terminal or C-terminal of the protein or truncated protein;
(3) the additional protein or polypeptide is selected from the group consisting of an epitope tag, a reporter gene sequence, a nuclear localization signal (NLS) sequence, a targeting moiety, a transcriptional activation domain, a transcriptional repression domain, a nuclease domain, and any combination thereof;
(4) the conjugate comprises an epitope tag; and,
(5) the conjugate comprises an NLS sequence.

6. A fusion protein, which comprises the protein according to claim 1 or a truncated protein comprising the protein and an additional protein or polypeptide.

7. The fusion protein according to claim 6, the fusion protein is characterized by one or more of the following:
(1) the additional protein or polypeptide is connected to the N-terminal or C-terminal of the protein or truncated protein optionally via a linker;
(2) the additional protein or polypeptide is selected from the group consisting of epitope tag, reporter gene sequence, nuclear localization signal (NLS) sequence, targeting moiety, transcription activation domain, transcription repression domain, nuclease domain, and any combination thereof;
(3) the fusion protein comprises an epitope tag; and,
(4) the fusion protein comprises an NLS sequence.

8. The fusion protein according to claim 7, the fusion protein is characterized by one or more of the following:
(1) the NLS sequence is set forth in SEQ ID NO: 27;
(2) the NLS sequence is located at, near or close to the N-terminal or C-terminal of the protein or truncated protein;
(3) the fusion protein has an amino acid sequence as set forth in any one of SEQ ID NO: 28 to 30.

9. An isolated nucleic acid molecule, which comprises: a nucleotide sequence encoding the protein according to claim 1, or a truncated protein comprising the protein, or a fusion protein comprising the protein.

10. A vector, which comprises the isolated nucleic acid molecule according to claim 9.

11. A host cell, which comprises the isolated nucleic acid molecule according to claim 9 or a vector comprising the isolated nucleic acid molecule.

12. A composition or complex, which comprises:
(i) a first component, which is selected from the group consisting of the protein according to claim 1, a truncated protein comprising the protein, a conjugate comprising the protein, a fusion protein comprising the protein, a nucleotide sequence encoding the protein, truncated protein or fusion protein, and any combination thereof; and
(ii) a second component, which is a nucleotide sequence comprising a guide RNA, or a nucleotide sequence encoding the nucleotide sequence comprising a guide RNA;
wherein, the guide RNA comprises a direct repeat sequence and a guide sequence from the 5′ to 3′ direction, and the guide sequence is capable of hybridizing with a target sequence;
the guide RNA is capable of forming a complex with the protein, truncated protein, conjugate or fusion protein as described in (i).

13. The composition or complex according to claim 12, the composition or complex is characterized by one or more of the following:
(1) the guide sequence is linked to the 3′ end of the direct repeat sequence; for example, the guide sequence comprises a complementary sequence of the target sequence;
(2) the composition or complex does not comprise a trans-activating crRNA (tracrRNA).

14. A composition or complex, which comprises one or more vectors, wherein the one or more vectors comprise:
(i) a first nucleic acid, which comprises a nucleotide sequence encoding the protein according to claim 1, a truncated protein comprising the protein, or a fusion protein comprising the protein; optionally, the first nucleic acid is operably ligated to a first regulatory element; and
(ii) a second nucleic acid, which comprises a nucleotide sequence encoding a guide RNA; optionally, the second nucleic acid is operably ligated to a second regulatory element;
wherein:
the first nucleic acid and the second nucleic acid are present on the same vector or different vectors;
the guide RNA comprises a direct repeat sequence and a guide sequence from the 5′ to 3′ direction, and the guide sequence is capable of hybridizing with a target sequence;
the guide RNA is capable of forming a complex with the protein, truncated protein or fusion protein as described in (i).

15. The composition or complex according to claim 12, wherein, when the target sequence is DNA, the target sequence is located at the 3′ end of a protospacer adjacent motif (PAM), and the PAM has a sequence shown as 5′-RYR, wherein R is A or G, and Y is T or C;
preferably, the sequence of the PAM is selected from the group consisting of ATG, ACG, GTG, ATA, ACA, GCA, GTA and/or GCG.

16. The composition or complex according to claim 12, wherein, the target sequence is a DNA or RNA sequence derived from a prokaryotic cell or a eukaryotic cell; or, the target sequence is a non-naturally occurring DNA or RNA sequence.

17. The composition or complex according to claim 12, wherein, the target sequence is present in a cell; or, the target sequence is present in a nucleic acid molecule (e.g., a plasmid) in vitro;
for example, the target sequence is present in a cell nucleus or cytoplasm (e.g., an organelle); for example, the cell is a prokaryotic cell; for example, the cell is a eukaryotic cell.

18. The composition or complex according to claim 12, wherein, the protein or truncated protein is linked to one or more NLS sequences, or, the conjugate or fusion protein comprises one or more NLS sequences;
for example, the NLS sequence is linked to the N-terminal or C-terminal of the protein or truncated protein; for example, the NLS sequence is fused to the N-terminal or C-terminal of the protein or truncated protein.

19. A kit, which comprises one or more components selected from the following: the protein according to claim 1, a truncated protein comprising the protein, a conjugate comprising the protein, a fusion protein comprising the protein, an isolated nucleic acid molecule comprising a nucleotide sequence encoding the protein, an isolated nucleic acid molecule comprising a nucleotide sequence encoding the protein, a vector comprising the protein, a composition or complex comprising the protein.

20. A delivery composition, which comprises a delivery vector and one or more selected from the following: the protein according to claim 1, a truncated protein comprising the protein, a conjugate comprising the protein, a fusion protein comprising the protein, an isolated nucleic acid molecule comprising a nucleotide sequence encoding the protein, an isolated nucleic acid molecule comprising a nucleotide sequence encoding the protein, a vector comprising the protein, and a composition or complex comprising the protein.

21. The delivery composition according to claim 20, the delivery composition is characterized by one or more of the following:
(1) the delivery vector is a particle;
(2) the delivery vector is selected from the group consisting of lipid particle, sugar particle, metal particle, protein particle, liposome, exosome, microvesicle, gene gun or viral vector.

22. A method for modification of a target gene, which comprises: contacting a composition or complex comprising the protein with the target gene, or delivering it to a cell containing the target gene; wherein, the target sequence is present in the target gene.

23. The method according to claim 22, the method is characterized by one or more of the following:
(1) the target gene is present in a cell, or, the target gene is present in a nucleic acid molecule (for example, a plasmid) in vitro;
(2) the cell is a prokaryotic cell; for example, the cell is a eukaryotic cell; for example, the cell is selected from the group consisting of animal cell (e.g., mammalian cell, such as human cell), plant cell;
(3) the modification refers to a break in the target sequence, such as a double-strand break in DNA or a single-strand break in RNA; and,
(4) the modification further comprises inserting an exogenous nucleic acid into the break.

24. A method for changing the expression of a gene product, which comprises: contacting a composition or complex comprising the protein with a nucleic acid molecule encoding the gene product, or delivering it to a cell containing the nucleic acid molecule; wherein, the target sequence is present in the nucleic acid molecule.

25. The method according to claim 24, the method is characterized by one or more of the following:
(1) the nucleic acid molecule is present in a cell, or the nucleic acid molecule is present in a nucleic acid molecule (for example, a plasmid) in vitro;
(2) the cell is a prokaryotic cell; for example, the cell is a eukaryotic cell; for example, the cell is selected from the group consisting of animal cell (e.g., mammalian cell, such as human cell), plant cell;
(3) the expression of the gene product is changed (e.g., enhanced or reduced); for example, the gene product is a protein;
(4) the protein, truncated protein, conjugate, fusion protein, isolated nucleic acid molecule, vector or composition or complex is contained in a delivery vehicle;
for example, the delivery vehicle is selected from the group consisting of lipid particle, sugar particle, metal particle, protein particle, liposome, exosome, viral vector; and,
(5) the method is used to modify a cell, cell line or organism by changing one or more target sequences in a target gene or a nucleic acid molecule encoding a target gene product.

26. A cell or progeny thereof obtained by the method according to claim 20, wherein the cell comprises a modification that is not present in its wild type.

27. A cell product of the cell or progeny thereof according to claim 26.

28. An in vitro, ex vivo or in vivo cell or cell line or progeny thereof, wherein the cell or cell line or progeny thereof comprises: the protein according to claim 1, a truncated protein comprising the protein, a conjugate comprising the protein, a fusion protein comprising the protein, an isolated nucleic acid molecule comprising a nucleotide sequence encoding the protein, an isolated nucleic acid molecule comprising a nucleotide sequence encoding the protein, a vector comprising the protein, a composition or complex comprising the protein;
for example, the cell is a prokaryotic cell or a eukaryotic cell.

29. A method for detecting whether a target nucleic acid is present in a sample, comprising the following steps:
(1) contacting the sample with a labeled DNA probe and any of the following components: a composition or complex comprising the protein, or a kit comprising the protein;
wherein, the guide sequence contained in the composition or complex, or kit is capable of hybridizing with the target nucleic acid, and the DNA probe is not capable of hybridizing with the guide sequence; preferably, the DNA probe emits a detectable signal after being cleaved;
(2) detecting the detectable signal generated by the cleavage of DNA probes by the protein or truncated protein contained in the composition or complex, or kit, thereby determining whether the target nucleic acid is present in the sample;
preferably, one end (e.g., the 5′ end) of the DNA probe is labeled with a fluorescent group, and the other end (e.g., the 3′ end) is labeled with a quenching group.

30. The method according to claim 29, the method is characterized by one or more of the following:
(1) wherein the sequence of the target nucleic acid is a sequence obtained from a pathogen; preferably, the pathogen is selected from the group consisting of a virus, a bacterium, a fungus, a protozoa, a parasite or any combination thereof;
(2) the sequence of the target nucleic acid is obtained from the genome of a tumor cell;
(3) the method further comprises a step of contacting the sample with a reagent for reverse transcription; preferably, the reagent for reverse transcription is selected from the group consisting of reverse transcriptase, oligonucleotide primer, dNTP or any combination thereof;
(4) the target nucleic acid is single-stranded or double-stranded; preferably, the sequence of the target nucleic acid is a DNA or RNA sequence derived from a prokaryotic cell or a eukaryotic cell; or, the sequence of the target nucleic acid is a non-naturally occurring DNA or RNA sequence;
(5) the detectable signal is determined by one or more methods selected from the following: imaging-based detection, sensor-based detection, color detection, gold nanoparticle-based detection, fluorescence polarization, colloidal phase transition/dispersion, electrochemical detection and semiconductor-based sensing;
(6) the method further comprises a step of amplifying the target nucleic acid in the sample.
